(19) United States
(12) Patent Application Publication
(10) Pub. No.: US 2005/0282859 A1
(43) Pub. Date: Dec. 22, 2005

(54) Dual acting SNRI-NMDA antagonists for the treatment of genitourinary disorders

(76) Inventor: Karl Bruce Thor, Cary, NC (US)

Correspondence Address: LAHIVE & COCKFIELD, LLP., 28 STATE STREET, BOSTON, MA, 02109, US

(21) Appl. No.: 11/145,022

(22) Filed: Jun. 3, 2005

Related U.S. Application Data
(60) Provisional application No. 60/576,999, filed on Jun. 4, 2004.
(60) Provisional application No. 60/607,820, filed on Sep. 7, 2004.
(60) Provisional application No. 60/640,105, filed on Dec. 28, 2004.

(51) Int. Cl. A61K 31/445; A61K 31/403; A61K 31/165; A61K 31/138

(52) U.S. Cl. 514/317; 514/412; 514/620; 514/649; 514/408; 514/454; 514/651

(57) ABSTRACT

Discolsed herein are compositions and methods for treatment of genitourinary disorders (e.g., urge incontinence). The compositions may generally include a dual-acting SNRI-NMDA antagonist (e.g., bicifadine and/or milnacipran). Alternatively, the compositions may generally include an SNRI and an NMDA antagonist.

### RELATED APPLICATIONS

This application claims the benefit of and priority to U.S. Provisional Application No. 60/576,999, filed Jun. 4, 2004, U.S. Provisional Application No. 60/607,820, filed Sep. 7, 2004, and U.S. Provisional Application No. 60/640,105, filed Dec. 28, 2004. The entireties of these applications are hereby incorporated by this reference.

### BACKGROUND OF THE INVENTION

Lower urinary tract disorders affect the quality of life of millions of men and women in the United States every year. While the kidneys filter blood and produce urine, the lower urinary tract functions to store and periodically eliminate urine. A complex neural control system in the brain and spinal cord control these functions, and allow the synergy between the storage components (i.e., the urinary bladder) and the elimination components (i.e., the urethra and the urethral sphincter). Generally, the lower urinary tract includes all other parts of the urinary tract except the kidneys, e.g the ureters, the urinary bladder, sphincter and the urethra.

As such, the lower urinary tract is strongly interrelated with the genitourinary system, which also generally includes all organs involved in the formation and voidance of urine and the organs involved in reproduction. Disorders of the genitourinary system include, for example, overactive bladder, overactive bladder with sphincter dysfunction, urinary incontinence, urge urinary incontinence, stress urinary incontinence, Fowler's Syndrome, outlet obstruction, outlet insufficiency, pelvic hypersensitivity, sphincteric spasticity, detrusor hyperreflexia (neurogenic bladder), detrusor instability, benign prostatic hyperplasia (BPH), urethral stricture disease, tumors, interstitial (cell) cystitis, chronic pelvic pain syndrome, prostatodynia, prostatis, vulvodynia, vulvar vestibulitis, urethritis, and/or orchidalgia.

Overactive bladder is a treatable medical condition that is estimated to affect 17 to 20 million people in the United States. Symptoms of overactive bladder can include urinary frequency, urinary urgency, urinary urge incontinence (accidental loss of urine) due to a sudden and unstoppable need to urinate, nocturia (the disturbance of nighttime sleep because of the need to urinate) or enuresis resulting from overactivity of the detrusor muscle (the smooth muscle of the bladder which contracts and causes it to empty).

Neurogenic overactive bladder (or neurogenic bladder) is a type of overactive bladder which occurs as a result of detrusor muscle overactivity referred to as detrusor hyperreflexia, secondary to known neurologic disorders. Patients with neurologic disorders, such as stroke, Parkinson's disease, diabetes, multiple sclerosis, peripheral neuropathy, or spinal cord lesions often suffer from neurogenic overactive bladder. In contrast, non-neurogenic overactive bladder occurs as a result of detrusor muscle overactivity referred to as detrusor muscle instability. Detrusor muscle instability can arise from non-neurological abnormalities, such as bladder stones, muscle disease, urinary tract infection or drug side effects or can be idiopathic.

Due to the enormous complexity of micturition (the act of urination) an exact mechanism which causes overactive bladder is not known. Overactive bladder can result from hypersensitivity of sensory neurons of the urinary bladder, arising from various factors including inflammatory conditions, hormonal imbalances, and prostate hypertrophy. Destruction of the sensory nerve fibers, either from a crushing injury to the sacral region of the spinal cord, or from a disease that causes damage to the dorsal root fibers as they enter the spinal cord can also lead to overactive bladder. In addition, damage to the spinal cord or brain stem causing interruption of transmitted signals can lead to abnormalities in micturition. Therefore, both peripheral and central mechanisms can be involved in mediating the altered activity in overactive bladder.

In spite of the uncertainty regarding whether central or peripheral mechanisms, or both, are involved in overactive bladder, many proposed mechanisms implicate neurons and pathways that mediate non-painful and painful visceral and pelvic somatic sensation. Viscerosensory information from the bladder and somatosensory information from the pelvic region is relayed by Aδ and nociceptive C fibers that enter the spinal cord via the dorsal root ganglion (DRG) and project to the brainstem and thalamus via second or third order neurons (Andersson (2002) Urology 59:18-24; Andersson (2002) Urology 59:43-50; Morrison, J., Steers, W. D., Brading, A., Blok, B., Fry, C., de Groat, W. C., Kakizaki, H., Levin, R., and Thor, K. B., “Basic Urological Sciences” In: Incontinence (vol. 2) Abrams, P. Khoury, S., and Wein, A. (Eds.) Health Publications, Ltd., Plymbridge Ditributors, Ltd., Plymouth, UK., (2002)). A number of different subtypes of sensory afferent neurons can be involved in neurotransmission from the lower urinary tract. These can be classified as, but not limited to, small diameter, medium diameter, large diameter, myelinated, unmyelinated, sacral, lumbar, peptidergic, non-peptidergic, IB4 positive, IB4 negative, C fiber, Aδ fiber, high threshold or low threshold neurons. Nociceptive input to the DRG is thought to be conveyed to the brain along several ascending pathways, including the spinothalamic, spinoreticular, spinomesencephalic, spinocervical, and in some cases dorsal column/medial lemniscal tracts (A. I. Basbaum and T. M. Jessell (2000) The perception of pain. In Principles of Neural Science, 4th. ed.).

Stress urinary incontinence is the involuntary loss of urine with the increase in intra-abdominal pressure. The primary etiological factor producing genuine stress urinary incontinence is the incomplete transmission of abdominal pressure to the proximal urethra due to displacement from its intra-abdominal position. Some people, especially women who have given birth to one or more children, and older women, can experience incidences of involuntary urine loss due to stress urinary incontinence or combined stress and urge incontinence. A sneeze or cough increases the intra-abdominal pressure, which in turn increases the pressure on a person's bladder causing the involuntary release of urine. The frequency and severity of such urine loss can increase as the muscles and tissues near the urethro-vaginal myofascial area grow weaker. It has also been recognized that the urinary sphincter muscle, which is located at the upper end of the urethra, adjacent to the bladder, works well at sealing off the passing of urine from the bladder to the urethra when functioning correctly.

Although there are specialized products available for the reduction of involuntary urine loss, many are surgical or are devices that must be inserted, can only be purchased with a prescription, and need to be properly sized, physically inserted and/or adjusted by a medical doctor for them to correctly perform. Other methods for reducing involuntary urine loss include pelvic muscle exercises. Long term success, however, is low because they must be performed regularly and the rate of discontinuation is high. Medications such as antimuscarinic drugs, which are useful in urinary incontinence, are not effective for the treatment of stress urinary incontinence.

Currently there are no regulatory agency approved clinical applications of central nervous system oriented pharmacotherapies for treating lower urinary tract disorders, such as overactive bladder or stress urinary incontinence. However, recent animal studies have suggested potential targets in the central nervous system for modulating urinary tract functions. For example, in the raphe nucleus of the caudal brain stem, 5-hydroxytryptamine (serotonin, 5-HT) containing neurons send projections to the dorsal horn as well as to the autonomic and sphincter motor nuclei in the lumbosacral spinal cord. The sympathetic and parasympathetic autonomic nuclei as well as the sphincter motor nuclei receive prominent serotonergic input from the raphe nuclei in the caudal brain stem. Activity in the serotonergic pathway generally enhances urine storage by facilitating the vesical sympathetic reflex pathway and inhibiting the parasympathetic voiding pathway (Sharma, A. et al. (2000) J. Clin. Pharmacol. 40: 161 and Thor, K. B. et al. (1995) J. Pharmacol. Exp. Ther. 274: 1016.) Among the various subtypes of 5-HT receptors, 5-HT₂ and 5-HT₃ receptors mediate excitatory effects on sympathetic and somatic reflexes to increase outlet resistance. Moreover, 5-HT_{2C} and 5-HT₃ receptors have also been shown to be involved in inhibition of the micturition reflex (Downie, J. W. (1999) Curr. Opin. SPNS Inves. Drugs 1:23). In fact, 5-HT₃ receptor inhibition has been shown to diminish 5-HT mediated contractions in rabbit detrusor (Khan, M. A. et al. (2000) Urol. Res. 28:116).

In addition to the role of serotonin in lower urinary tract physiology, norepinephrine has also been shown to have effects on lower urinary tract function. Norepinephrine has been shown to stimulate both β₃-adrenergic receptors and α₁-adrenergic receptors. β₃-adrenergic receptors allow relaxation of the bladder and α₁-adrenergic receptors allow contraction of the sphincter, which both facilitate the storage of urine. Central noradrenergic pathways also play a role in control of bladder and sphincter function Thor, K. B. et al. (1995) J. Pharmacol. Exp. Ther. 274: 1016.

Current treatments for overactive bladder include medication, diet modification, programs in bladder training, electrical stimulation, and surgery. Currently, antimuscarinics (which are members of the general class of anticholinergics) are the primary medication used for the treatment of overactive bladder. The antimuscarinic, oxbutynin, has been the mainstay of treatment for overactive bladder. However, treatment with antimuscarinics suffers from limited efficacy and side effects such as dry mouth, dry eyes, dry vagina, blurred vision, cardiac side effects, such as palpitations and arrhythmia, drowsiness, urinary retention, weight gain, hypertension and constipation, which have proven difficult for some individuals to tolerate.

Benign prostatic hyperplasia (BPH) is a non-malignant enlargement of the prostate that is very common in men over 40 years of age. BPH is thought to be due to excessive cellular growth of both glandular and stromal elements of the prostate. Symptoms of BPH can include urinary frequency, urinary urgency, urge incontinence, nocturia, or reduced urinary force and speed of flow.

Invasive treatments for BPH include transurethral resection of the prostate, transurethral incision of the prostate, balloon dilation of the prostate, prostatic stents, microwave therapy, laser prostatectomy, transrectal high-intensity focused ultrasound therapy and transurethral needle ablation of the prostate. However, complications can arise through the use of some of these treatments, including retrograde ejaculation, impotence, postoperative urinary tract infection and some urinary incontinence. Non-invasive treatments for BPH include androgen deprivation therapy and the use of 5α-reductase inhibitors and α-adrenergic blockers. However, these treatments have proven only minimally to moderately effective for some patients.

Chronic pelvic pain syndrome and associated prostatitis and prostatodynia are other lower urinary tract disorders that have been suggested to affect approximately 2-9% of the adult male population (Collins M M, et al., (1998) J. Urology, 159: 1224-1228). Currently, there are no established treatments for prostatitis and prostatodynia. Antibiotics are often prescribed, but with little evidence of efficacy. COX-2 selective inhibitors and α-adrenergic blockers and have been suggested as treatments, but their efficacy has not been established. Hot sitz baths and anticholinergic drugs have also been employed to provide some symptomatic relief.

Vulvodynia is chronic vulvar burning/pain without clear medical findings (L. Edwards, 2003, Am. J. Obst. & Gynecol. 189:S24-30). The etiology of vulvodynia is unknown and is divided into 2 classes: vulvar vestibulitis syndrome elicited by touch and dysesthetic vulvodynia not limited to the vestibule and may occur without touch or pressure. Unfortunately, clinical trials on potential vulvodynia therapies have been few. Standard therapy includes neuropathic pain treatments (e.g., tricyclic medications, gabapentin). Additional therapies may be considered: pelvic floor rehabilitation combined with surface electromyography, interferon alfa, estrogen creams, and surgery. However, definitive data on effective therapies are lacking.

### SUMMARY OF THE INVENTION

The present invention provides therapies and compositions useful in treating genitourinary disorders, e.g., dry overactive bladder and urge urinary incontinence. The methods of the present invention are generally carried out using a dual acting serotonin-norepinephrine reuptake inhibitor (SNRI)-N-methyl-D-aspartic acid (NMDA) antagonist, i.e., a dual acting SNRI-NMDA antagonist. In one embodiment, the dual acting SNRI-NMDA antagonist comprises an SNRI and an NMDA antagonist. In another embodiment, the dual acting SNRI-NMDA antagonist comprises one agent having both SNRI activity and NMDA antagonist activity. Without wishing to be bound by any particular theory, it is believed that, because serotonin-norepinephrine reuptake inhibitors and glutamate receptor antagonists, such as NMDA antagonists, are generally active against different pathways relating to micturition, the administration of both in a non-interfering manner would increase the benefit of the therapy.

A potential interference between an SNRI and glutamate antagonist lies in the dependence of an SNRI upon ongoing activity in serotonin and norepinephrine neurons to demonstrate an effect (i.e., if there is no release of serotonin or norepinephrine, then inhibiting reuptake has no effect) and the importance of glutamate as an excitatory transmitter for maintaining ongoing activity of norepinephrine neurons (Ennis, M., Aston-Jones, G., 1988. J. Neurosci. 8, 3644-3657) and serotonergic neurons (Tao R. Auerbach S B. Brain Research. 961(1):109-20, 2003). Thus, it was unexpected that antagonism of the NMDA receptor did not effectively reduce the activity of norepinephrine and serotonergic neurons to also inhibit the SNRI activity.

Accordingly, the invention relates to a method of treating a genitourinary disorder in a subject in need of treatment. The method comprises administering to the subject in need of treatment a therapeutically effective amount of a dual acting SNRI-NMDA antagonist, such that the genitourinary disorder is treated. In some embodiments, the dual acting SNRI-NMDA antagonist includes a first amount of an SNRI and a second amount of an NMDA antagonist. The first amount and the second amount can both be a therapeutically effective amount. Alternatively, the first amount and the second amount together form a therapeutically effective amount In other embodiments, the dual acting SNRI-NMDA antagonist includes one agent having both SNRI activity and NMDA antagonist activity.

In a particular embodiment, the dual acting SNRI-NMDA antagonists are 1-phenyl-3-azabicyclo[3.1.0]hexane derivatives such as those described in U.S. Pat. Nos. 4,131,611, 4,196,120, 4,231,955, and 4,435,419 the entire contents of which are incorporated herein by reference.

In specific embodiments, the dual acting SNRI-NMDA antagonist is a compound of Formula I:

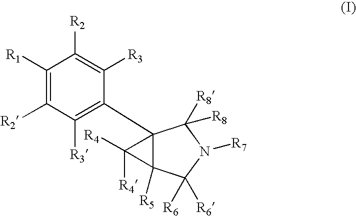

wherein R₁, R₂, R₂′, R₃, R₃′, R₄, R₄′, R₅, R₆, R₆′, R₇, R₈, and R₈′ are each independently H, alkyl, aryl, alkenyl, alkynyl, alkoxy, alkoxyalkyl, cycloalkyl, acyl, aroyl, carboxyl, carbonyl, amino, alkylamino, dialkylamino, nitro, halogen, hydroxyl, amido, acetamido, or trifluoromethyl; optionally R₄ and R₄′ together form ═O, ═S, ═NH or ═CH₂; optionally R₆ and R₆′ together form ═O, ═S, ═NH or ═CH₂; and optionally R₈ and R₈′ together form ═O, ═S, ═NH or ═CH₂ or pharmaceutically acceptable salts thereof.

In some embodiments, R₁, R₂, R₂′, R₃ and R₃′ are each independently H, alkyl, aryl, alkoxy, alkoxyalkyl, cycloalkyl, amino, nitro, acetamido, hydroxyl, trifluoromethyl or halogen, R₄, R₄′, R₅, R₆, R₆′, R₈, and R₈′ are each independently H, alkyl, aryl, alkoxy, alkoxyalkyl, cycloalkyl, halogen or hydroxyl, optionally R₆ and R₆′ together form ═O, ═S, ═NH or ═CH₂, optionally R₈ and R₈′ together form ═O, ═S, ═NH or ═CH₂, and R₇is H, alkyl, aryl, alkoxy, alkoxyalkyl, cycloalkyl, acyl, carboxyl or carbonyl.

In other embodiments, R₁, R₂, R₂′, R₃ and R₃′ are each independently H, C₁-C₆ alkyl, C₁-C₆ alkoxy or halogen; R₄, R₄′, R₅, R₆, R₆′, R₈, and R₈′ are each independently H, C₁-C₆ alkyl, halogen or hydroxyl; optionally R₆ and R₆′ together form ═O; and optionally R₈ and R₈′ together form ═O; and R₇is H or C₁-C₆ alkyl optionally substituted with aryl or substituted aryl.

In some embodiments, at least one of R₁, R₂, R₂′, R₃ and R₃′ is other than. hydrogen.

In some embodiments, R₁ and R₂′ are not both chlorine if R₂, R₃, R₃′, R₄, R₄′, R₅, R₆, R₆′, R₇, R₈, and R₈′ are hydrogen.

In other embodiments, the compound represented by formula I is a single enantiomer.

In another embodiment, the dual acting SNRI-NMDA antagonist is represented by one of the following structural formulas:

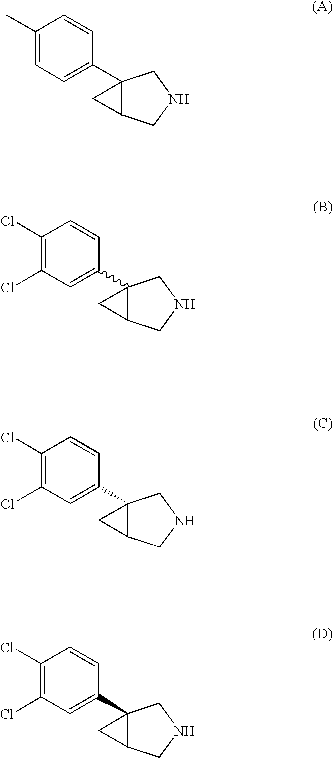

In another particular embodiment, the dual acting SNRI-NMDA antagonists are 2-(aminomethyl)-1-phenylcyclopropanecarboxamide derivatives, such as those described in U.S. Pat. Nos. 3,989,722, 4,478,836, 5,621,142, 6,602,911 and 6,635,675.

In another specific embodiment, the compounds having SNRI activity and NMDA antagonist activity are represented by Formula II:

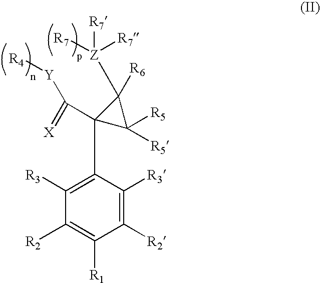

wherein X is selected from O, S and NR; Y is selected from O, S, and NR₄′; n and p are each independently 0 or 1; optionally R₇ and R₄ together form a direct bond between Y and Z; R, R₁, R₂, R₂′, R₃, R₃′, R₅, R₅′, R_{6,} R₇, and R₇′ are each independently aryl, heteroaryl, arylalkyl, alkenyl, alkynyl, alkoxy, alkoxyalkyl, aryloxy, arylalkyloxy, cycloalkyl, acyl, aroyl, carboxyl, carbonyl, amino, alkylamino, dialkylamino, arylamino, arylakylamino, nitro, halogen, hydroxyl, amido, acetamido, or trifluoromethyl, cyano, thio, alkylthio, arylthio, arylakylthio, azido, alkylseleno, formyl, silyl, silyloxy, (alkyloxy)carbonyl, (aryloxy)carbonyl, (arylalkyloxy)carbonyl, (alkylamino)carbonyl, (arylamino)carbonyl, (arylalkylamino) carbonyl, alkylsulfonyl, arylsulfonyl, or —(CH₂)ₘ—R₉; R₇″ is selected from H, NR₈R₈′, OR₈, and SR₈; R₉ is selected from aryl, cycloalkyl, heterocyclyl or polycyclyl; m is an integer from 0 to 8; R₄, R₄′, R₈, and R₈′ are each independently H, alkyl, aryl, aralkyl, alkenyl, alkynyl, alkoxyalkyl, cycloalkyl, acyl, aroyl, carboxyl, carbonyl; optionally R₄ and R₄′ together form a heterocyclic ring; optionally R₈ and R₈′ together form a heterocyclic ring; and optionally R₇ and R₇′ together form ═O, ═S, ═NH or ═CH₂; or pharmaceutically acceptable salts thereof.

In some embodiments, X is O; Y is O or NR₄′; n and p are each 1; R₁, R₂, R₂′, R₃, R₃′, are each independently H, alkyl, alkoxy, amino, nitro, halogen, or hydroxyl; R₄ and R₄′ are each independently H, alkyl, aryl, aralkyl, or optionally R₄ and R₄′ together form a heterocyclic ring; R₇″ is NR₈R₈′ or OR₈; R₈, and R₈′ are each independently H, alkyl, or optionally R₈ and R₈′ together form a heterocyclic ring; and R₅, R₅′, R₆, R₇, and R₇′ are each independently H, alkyl, or aryl. In some embodiments, n and p are each independently 0 and R₄ and R₇ together form a direct bond between Y and Z.

In other embodiments, R₁, R₂, R₂′, R₃ and R₃′ are H; X is O; Y is O or NR₄′; n and p are each 1; R₇″ is H, NR₈R₈′ or OR₈; R₄, R₄′, R₅, R₅′, R₆, R₇, R₇′, R₈, adn R₈′ are each independently H or C₁-C₆ alkyl. In still other embodiments, R₁, R₂, R₂′, R₃ and R₃′ are H; X is O; Y is O or NR₄′; n and p are each 0 and R₄ and R₇ form a direct bond between Y and Z; R₇′ is H, NR₈R₈′ or ORA; R₄′, R₅, R₅′, R₆, R₇′, R₈, and R₈′ are each independently H or C₁-C₆ alkyl.

In one embodiment, R₄ and R₄′ are each ethyl.

In other embodiments, the compound represented by formula II is a single enantiomer. In still further embodiments, the compound represented by formula II is not milnacipran.

In another embodiment, the the dual acting SNRI-NMDA antagonist is represented by one of the following structural formulas:

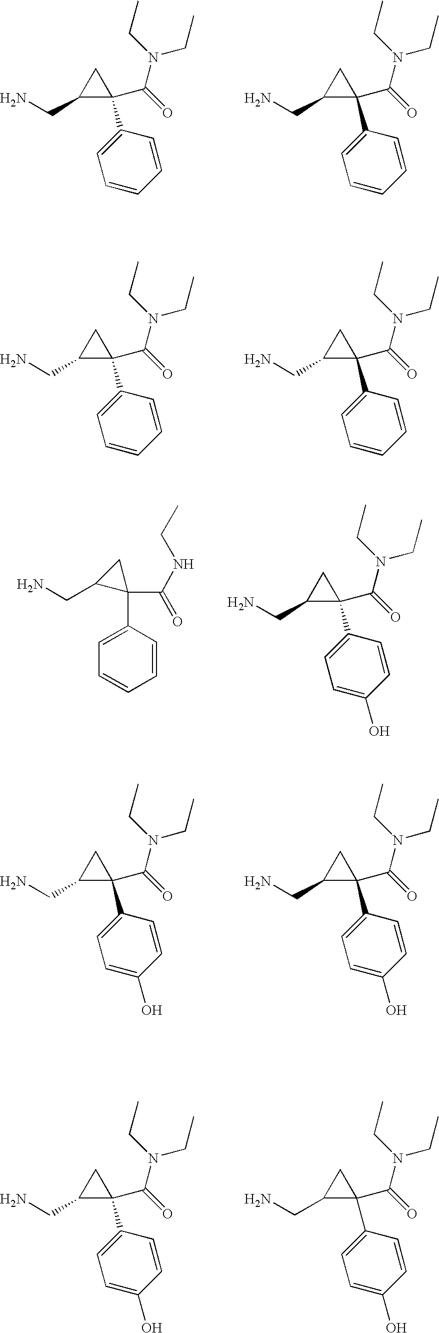

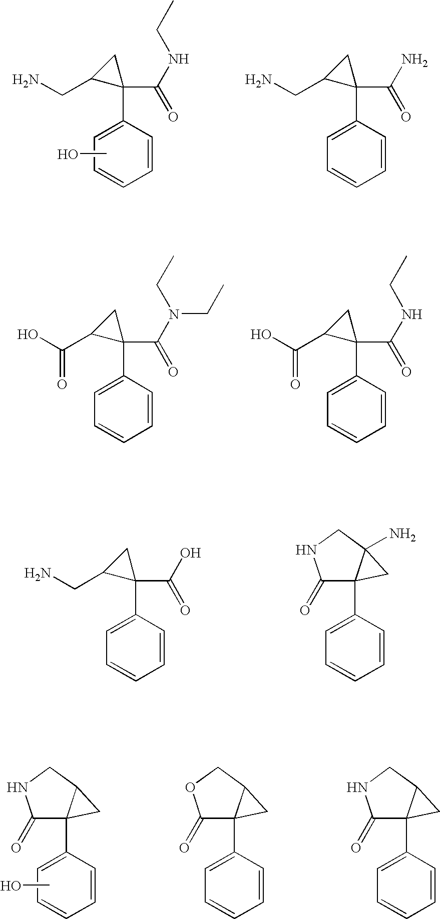

In still another particular embodiment, the dual acting SNRI-NMDA antagonists are diarylalkanamines and derivatives, such as those described in, for example, U.S. Pat. Nos. 3,510,560, 6,017,965, 6,071,970, and 6,211,245, and International Patent Application Numbers WO00/02551, WO98/56752, WO97/46511 and WO96/40097.

In specific embodiments, the dual acting SNRI-NMDA antagonist is a compound of Formula III:

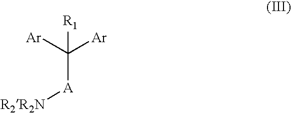

wherein each Ar is independently cycloalkyl, aryl, aralkyl, heteroaryl or heteroaralkyl group optionally substituted with one or more amino, alkylamino, dialkylamino, alkyl, hydroxyl, alkoxy, mercapto, alkylthio, alkylsulfinyl, acyl, halogen, perhaloalkyl, trifluoromethyl, trifluoromethylthio, trifluoromethylsulfonyl, and/or trifluoromethoxy; optionally each Ar is taken together to form a fused polycyclic ring system; R₁ is H, alkyl, aryl or aralkyl; R₂ and R₂′ are each independently H, alkyl, alkylaryl, acyl, or R₂ and/or R₂′ are taken together with A to form a heterocycle or a heteroaryl; and A is alkylene, alkenylene or alkynylene, optionally interrupted with —O—, —S—, or —NH—, or pharmaceutically acceptable sats thereof.

In some embodiments, each Ar is independently phenyl, phenoxy, benzyl, naphthyl, thiofuranyl, tetrahydronaphthyl, pyridyl, quinolinyl, isoquinolinyl, tetrahydroquinolinyl, tetrahydroisoquinolinyl, cyclohexyl, cycloheptyl or cyclopentyl, optionally substituted with one or more amino, alkylamino, dialkylamino, alkyl, hydroxyl, alkoxy, mercapto, alkylthio, alkylsulfinyl, acyl, halogen, perhaloalkyl, trifluoromethyl, trifluoromethylthio, trifluoromethylsulfonyl, and/or trifluoromethoxy; optionally each Ar is taken together to form a dibenzo[7]annulene, a dihydrodibenzo[7]annulene, a xanthenyl or a thioxanthenyl; R₁ is H or alkyl; R₂ and R₂′ are each independently H, alkyl or R₂ and/or R₂′ are taken together with A to form a heterocycle; and A is a liner or branched alkylene or alkenylene, optionally interrupted with an —O—.

In other embodiments, the compound represented by formula III is a single enantiomer.

In another embodiment, the dual acting SNRI-NMDA antagonist is represented by one of the following structural formulas:

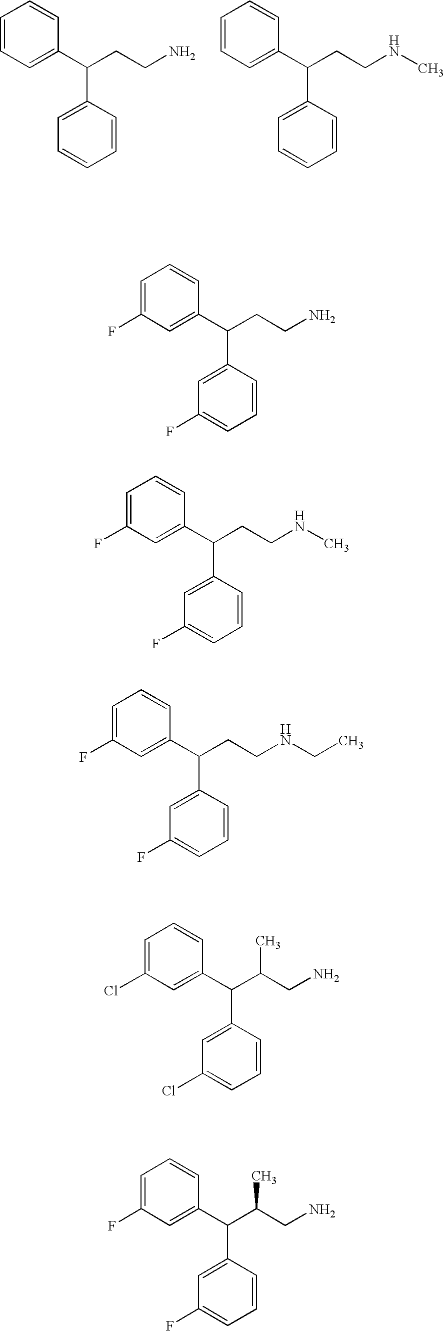

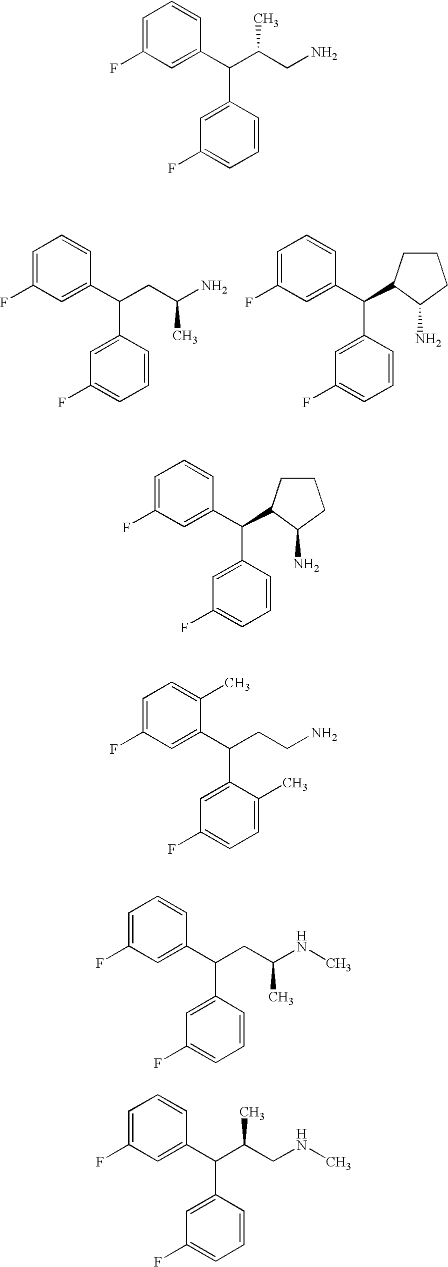

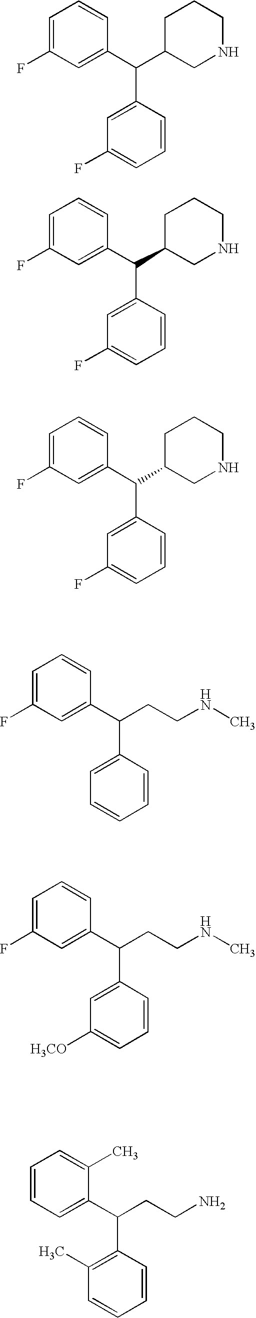

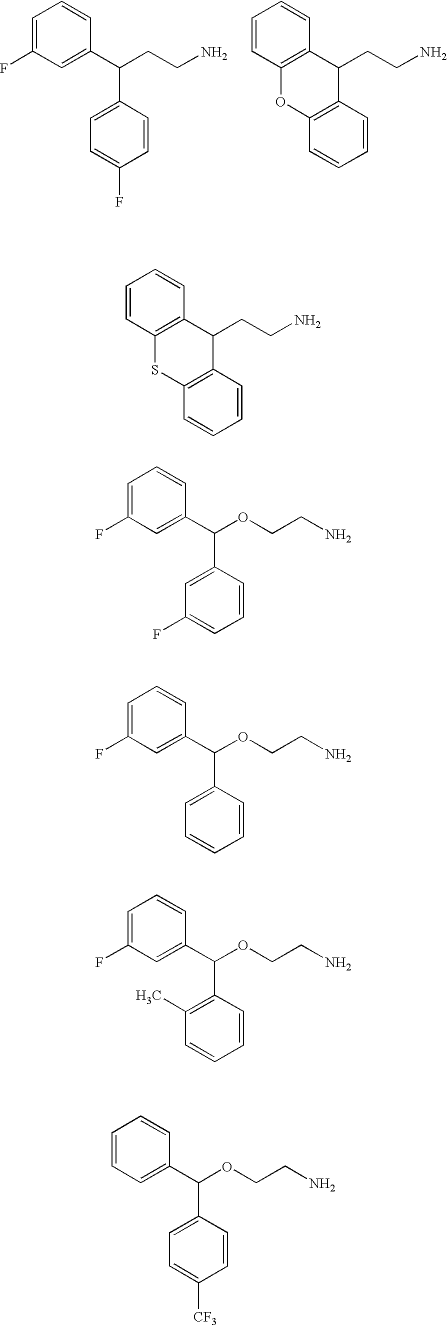

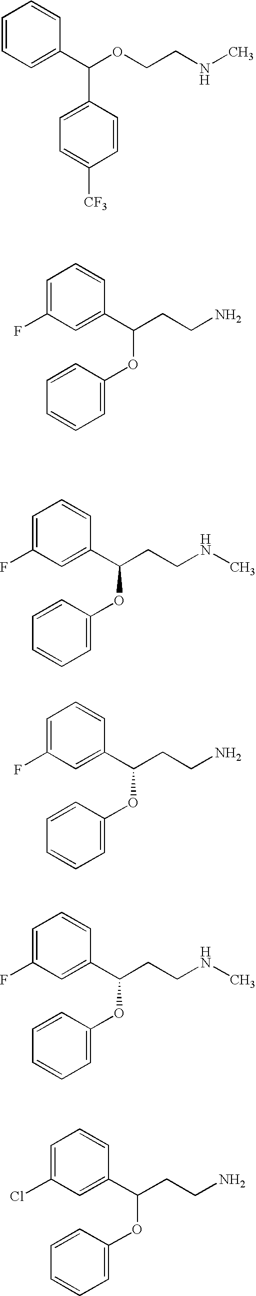

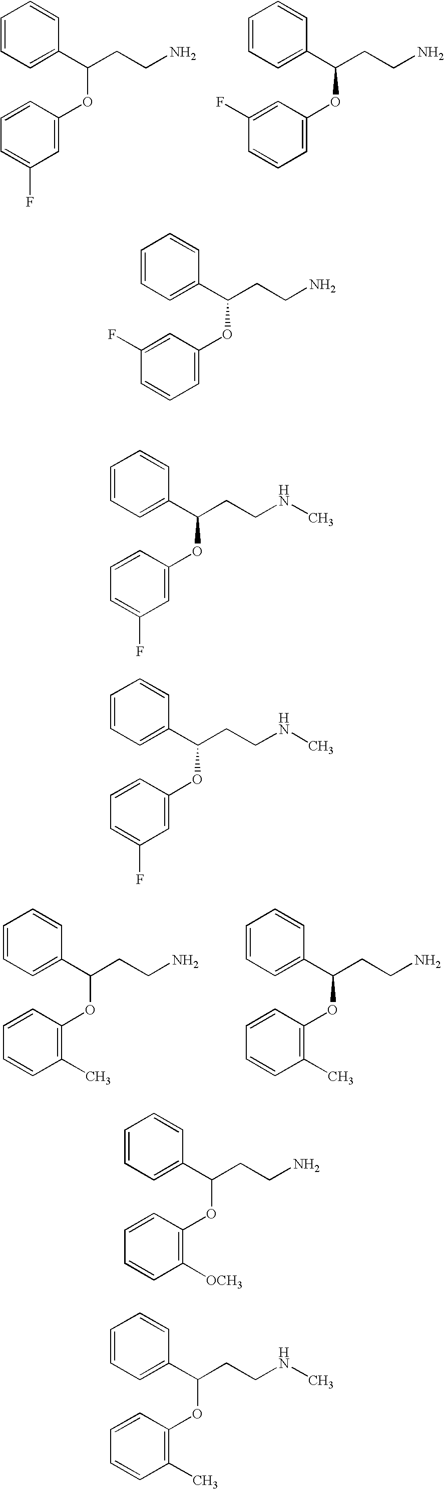

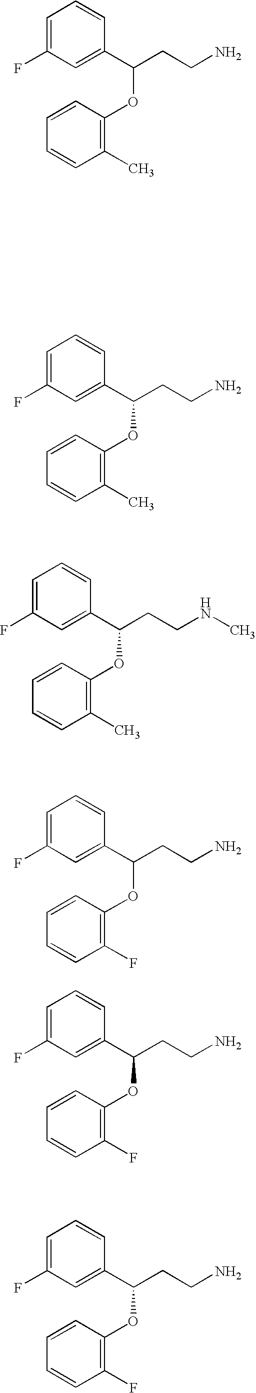

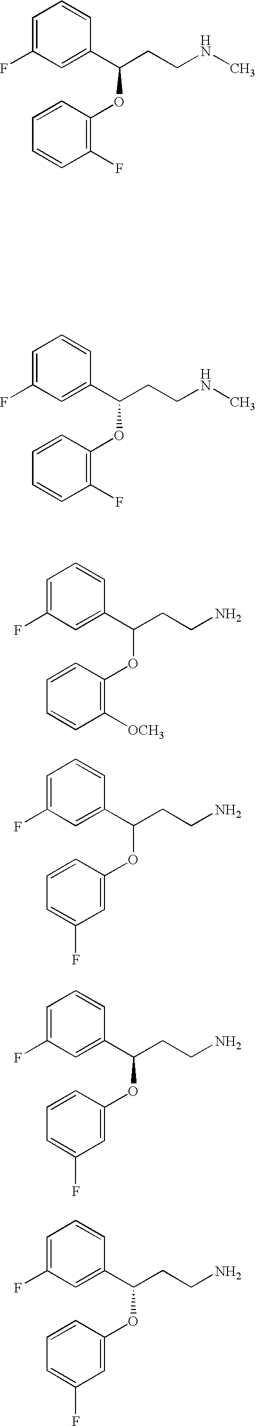

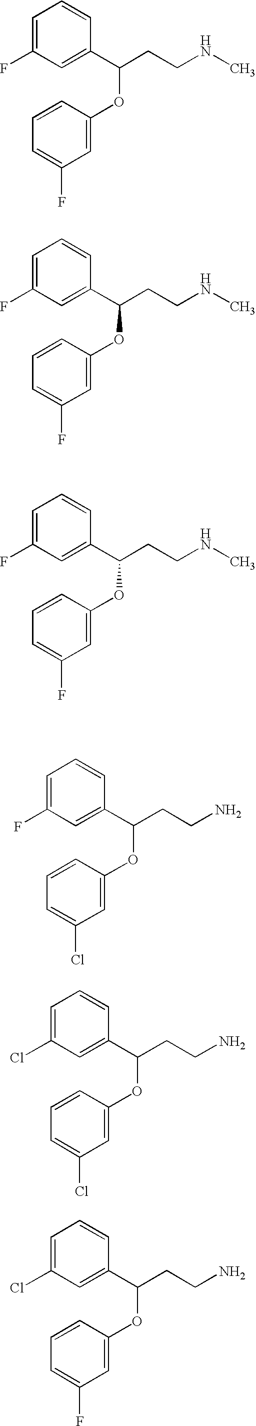

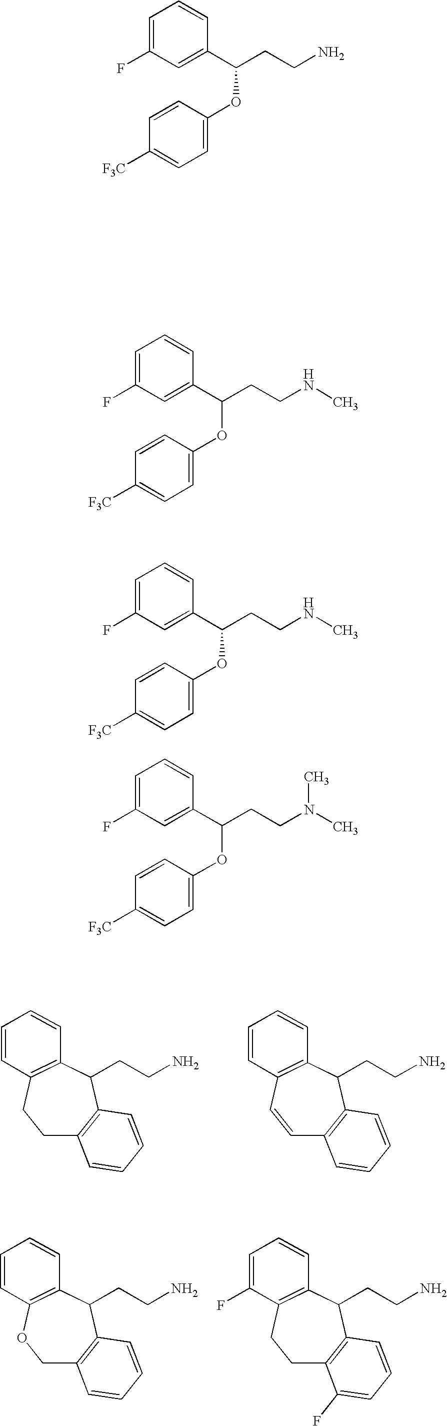

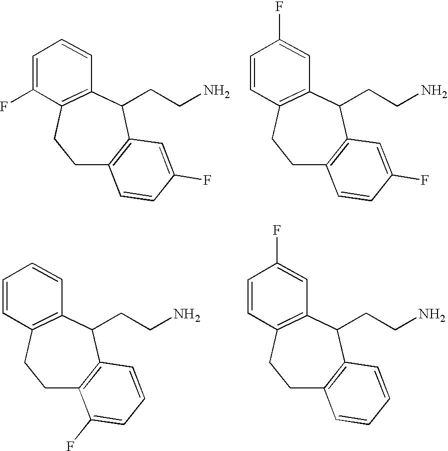

In some embodiments, the genitourinary disorder is a disorder associated with control of the smooth muscle of the urinary bladder, e.g., urge incontinence. In other embodiments, the genitourinary disorder is a disorder associated with control of the striated muscle of the urethral sphincter, e.g., stress urinary incontinence.

The genitourinary disorder can be, but is not limited to, overactive bladder, overactive bladder with sphincter dysfunction, urinary incontinence, urge urinary incontinence, stress urinary incontinence, Fowler's Syndrome, outlet obstruction, outlet insufficiency, pelvic hypersensitivity, sphincteric spasticity, detrusor hyperreflexia (neurogenic bladder), detrusor instability, benign prostatic hyperplasia (BPH), urethral stricture disease, tumors, interstitial (cell) cystitis, chronic pelvic pain syndrome, prostatodynia, prostatis, vulvodynia, vulvar vestibulitis, urethritis, and/or orchidalgia. In some embodiments, the genitourinary disorder is characterized by bladder-sphincter dissynergia. In another embodiment, the genitourinary disorder is a genitourinary disorder with outlet obstruction. In other embodiments, the genitourinary disorder is dry overactive bladder, overactive bladder with sphincter dysfunction, urge urinary incontinence, Fowler's Syndrome, chronic pelvic pain syndrome, prostatitis, prostatodynia, vulvodynia, vestibulitis, and/or benign prostatic hyperplasia.

In some embodiments, the dual acting SNRI-NMDA antagonist is administered on an as-needed basis. In preferred embodiments, the dual acting SNRI-NMDA antagonist may be administered prior to commencement of an activity wherein suppression of a genitourinary disorder is desired. For example, from about 0 minutes to about 10 hours prior to commencement of an activity wherein suppression of a genitourinary disorder is desired, or more preferably from about 0 minutes to about 3 hours prior to commencement of an activity wherein suppression of a genitourinary disorder is desired.

In some embodiments, the dual acting SNRI-NMDA antagonist is administered in a controlled release formulation, for example a delayed release formulation, a pulsatile release formulation, and/or a sustained release formulation.

In some embodiments, the dual acting SNRI-NMDA antagonist is administered orally. Oral administration can be, e.g., in the form of a tablet, a capsule, a caplet, a pill, a gel cap, a troche, a lozenge, a magma, a dispersion, a solution, a suspension, a syrup, a granule, a bead, a powder and/or a pellet. In some preferred embodiments, the dosage form is a tablet. In other preferred embodiments, the dosage form is a capsule.

In other embodiments, the dual acting SNRI-NMDA antagonist is administered transmucosally, e.g., sublingually, buccally, transurethrally, and/or rectally. In still other embodiments, the dual acting SNRI-NMDA antagonist is administered by inhalation, intravesically, topically, transdermally, and/or parenterally.

In some embodiments, the treatment does not result in unwanted side effects. In some embodiments, the heart rate of the subject being treated is not increased by more than 50%, preferably not more than 25%, more preferably not more than 10%. In other embodiments, the arterial pressure of the subject is not increased by more than 25%, preferably not more than 10%.

In some embodiments, the method further includes the administration to the subject a therapeutically effective amount of an antimuscarinic, oxybutynin, DITROPAN®, tolterodine, flavoxate, propiverine, trospium, a muscosal surface protectant, ELMIRON®, an antihistamine, hydroxyzine hydrochloride, pamoate, an anticonvulsant, NEURONTIN®, KLONOPIN®, a muscle relaxant, VALIUM®, a bladder antispasmodic, URIMAX®, a tricyclic antidepressant, imipramine, a nitric oxide donor, nitroprusside, a β₃-adrenergic receptor agonist, a bradykinin receptor antagonist, a neurokinin receptor antagonist, a sodium channel modulator, such as TTX-R sodium channel modulator and/or activity dependent sodium channel modulator and/or a Cav2.2 subunit calcium channel modulator.

In preferred embodiments, the present invention is directed to a method of treating a genitourinary disorder in a subject in need of treatment, which includes administering to the subject a therapeutically effective amount of at least one of the compounds with the following structural formulas:

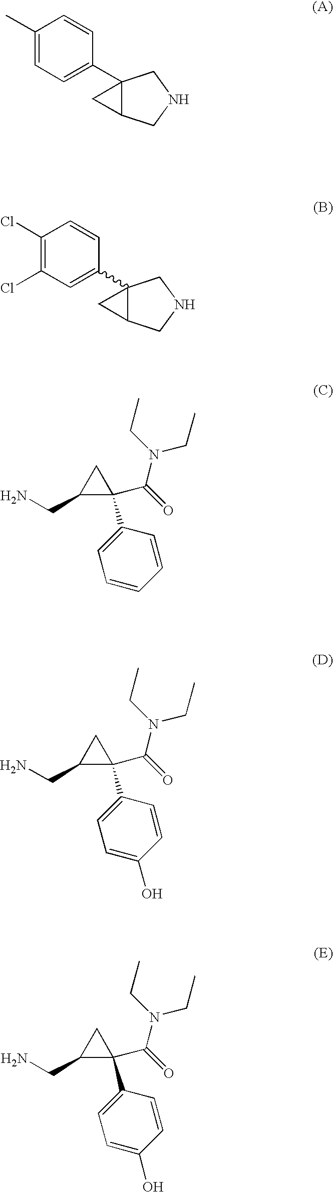

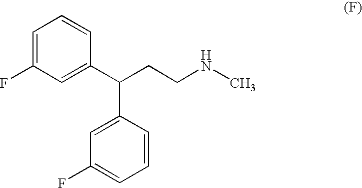

or pharmaceutically acceptable salts thereof, such that the genitourinary disorder is treated.

Preferably the subject is a human. Preferably, the subject does not have a chemical dependency.

The invention still further relates to a method of treating overactive bladder in a subject. In some embodiments, the method generally includes administering to the subject a therapeutically effective amount of a dual acting SNRI-NMDA antagonist, such that the overactive bladder is treated.

In certain embodiments, the method generally includes administering to the subject a therapeutically effective amount of at least one of the compounds with the following structural formulas:

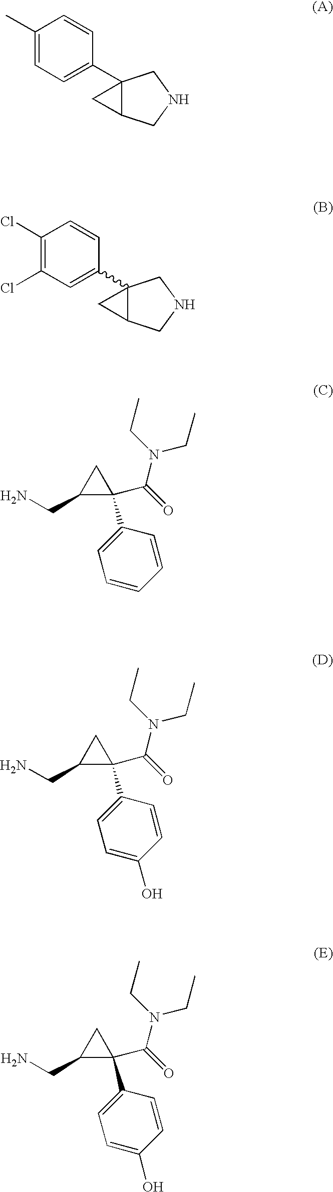

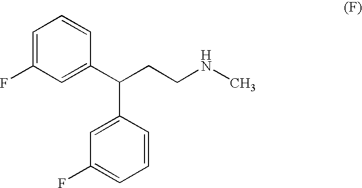

or pharmaceutically acceptable salts thereof, such that the overactive bladder is treated.

The invention further relates to pharmaceutical compositions useful for the treatment of a genitourinary disorder in a subject in need of treatment. The pharmaceutical composition comprises a therapeutically effective amount of a dual acting SNRI-NMDA antagonist and, optionally, a pharmaceutically acceptable carrier.

The invention further relates to the use of a dual acting SNRI-NMDA antagonist for the manufacture of a medicament for treating at least one symptom associated with a genitourinary disorder in a subject in need of treatment. The pharmaceutical composition used for the manufacture of a medicament can optionally contain a pharmaceutically acceptable carrier.

The invention further relates to a kit for treating a genitourinary disorder in a subject in need of treatment. The kit may include a dual acting SNRI-NMDA antagonist, packaged with instructions for using the dual acting SNRI-NMDA antagonist for the treatment of the genitourinary disorder. In one embodiment, the dual acting SNRI-NMDA antagonist may include a first amount of an SNRI and a second amount of an NMDA antagonist. In another embodiment, the dual acting SNRI-NMDA antagonist includes one agent having both SNRI activity and NMDA antagonist activity.

Alternatively, the kit may include an SNRI packaged with instructions for using the SNRI together with an NMDA antagonist for the treatment of a genitourinary disorder. The kit may otherwise include an NMDA antagonist packaged with instructions for using the NMDA antagonist together with an SNRI for the treatment of a genitourinary disorder.

The invention further relates to a method for processing a claim under a health insurance policy submitted by a claimant seeking reimbursement for costs associated with treatment of a genitourinary disorder with a therapeutically effective amount of a dual acting SNRI-NMDA antagonist. The method includes reviewing the claim, determining whether the treatment is reimbursable under the insurance policy, and processing the claim to provide partial or complete reimbursement of the costs.

The foregoing and other objects, features and advantages of the invention will be apparent from the following more particular description of preferred embodiments of the invention.

### BRIEF DESCRIPTION OF THE DRAWINGS

FIG. 1 is a set of graphs depicting the effect of cumulative increasing doses of bicifadine on bladder capacity in a rat. Data are normalized to irritation controls and are presented as Mean±SEM (left) and box plots (right).

FIG. 2 is a set of graphs depicting the effect of cumulative increasing doses of bicifadine on voiding efficiency. Data are presented as Mean±SEM (left) and box plots (right).

FIG. 3 is a physiograph tracing from a single experiment showing a bladder cystometrogram under conditions of saline infusion, 0.5% acetic acid infusion, acetic acid infusion 15 minutes post a 3 mg/kg i.v dose and acetic acid infusion 15 minutes post a 10 mg/kg i.v. dose of bicifadine.

FIG. 4 is a graph depicting bladder capacity expressed in mls during saline infusion (S), acetic acid infusion (AA) and effects of vehicle (Veh) or cumulative doses of bicifadine mg/kg i.v. P=0.0008 by Friedman test vehicle thru 10 mg/kg. (*p<0.05 and **p<0.01 vs. vehicle by Dunn's MC test).

FIG. 5 is a graph depicting contraction frequency (expressed as % of acetic acid control values) during saline infusion (S), acetic acid infusion (AA) and effects of vehicle (Veh) or cumulative doses of bicifadine mg/kg i.v. P=0.0435 by Friedman test vehicle thru 10 mg/kg.

FIG. 6 is a graph depicting mean arterial pressure (expressed as % of acetic acid control levels) during saline infusion (S), acetic acid infusion (AA) and effects of vehicle (Veh) or cumulative doses of bicifadine mg/kg i.v.

FIG. 7 is a graph depicting heart rate before and after drug (expressed as % of acetic acid control levels) during saline infusion (S), acetic acid infusion (AA) and effects of vehicle (Veh) or cumulative doses of bicifadine mg/kg i.v.

FIG. 8 is a physiograph tracing from a single experiment showing a bladder cystometrogram and external urethral sphincter EMG under conditions of saline infusion, 0.5% acetic acid infusion, and acetic acid infusion 15 minutes post a 10 mg/kg i.v. dose of milnacipran.

### DETAILED DESCRIPTION OF THE INVENTION

The invention relates to a method of treating at least one symptom associated with a genitourinary disorder in a subject in need of treatment.

The present invention is based, at least in part, on the discovery that certain compounds and combinations of compounds possess both SNRI activity and NMDA antagonist activity, wherein the SNRI activity and the NMDA antagonist activity do not interfere with each other. For example, the activity of the SNRI to contract the sphincter does not interfere with the ability of the NMDA antagonist to facilitate sphincter relaxation. The present invention is also based, at least in part, on the discovery that certain compounds and combinations of compounds which possess both SNRI activity and NMDA antagonist activity do not have a deleterious effect on the subject, e.g., arterial pressure or heart rate. Thus, in one embodiment, the heart rate of the subject is not increased by more than 50%. In a preferred embodiment, the heart rate of the subject is not increased by more than 25%, 20%, 15%, 10% . . . 1%. In another embodiment, the arterial pressure of the subject is not increased by more than 25%. In a preferred embodiment, the arterial pressure of the subject is not increased by more than 20%, 15%, 10% . . . 1%.

It is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

It must be noted that as used in this specification and the appended embodiments, the singular forms “a,” “an,” and “the” include plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

Furthermore, it is also to be understood that all of the numerical values presented in the present application are understood to be modified by the term “about.” That is, for example, a dosage of “100 mg” includes both the exact value of 100 mg and also “about 100 mg.” Accordingly, unless indicated to the contrary, the numerical parameters set forth in the specification and attached claims may vary depending upon the desired properties sought to be obtained by the subject matter presented herein. As used herein, the term “about” means that number referred to as having “about” comprises the recited number plus or minus up to 10% of that number.

Monoamine Neurotransmitters

Monoamine neurotransmitters such as noradrenaline (also referred to as norepinephrine), serotonin (5-hydroxytryptamine, 5-HT) and dopamine are known and disturbances in these neurotransmitters have been indicated in many types of disorders, such as depression. These neurotransmitters travel from the terminal of a neuron across a small gap referred to as the synaptic cleft and bind to receptor molecules on the surface of a second neuron. This binding elicits intracellular changes that initiate or activate a response or change in the postsynaptic neuron. Inactivation occurs primarily by transport of the neurotransmitter back into the presynaptic neuron, which is referred to as reuptake. These neurons or neuroendocrine cells can be found both in the Central Nervous System (CNS) and in the Peripheral Nervous System (PNS). It has also been shown that, in addition to numerous other functions, the synergy between storage and elimination of urine is modulated by a number of neurotransmitters, including monoamine transmitters.

Serotonin-Norepinephrine Reuptake Inhibitors

As used herein, the term serotonin-norepinephrine reuptake inhibitors (SNRI) refers to an agent (e.g., a molecule, a compound) which can inhibit the reuptake of both serotonin and norepinephrine. As such, inhibition of the reuptake of serotonin and norepinephrine in a subject can result in an increase in the concentration of physiologically active serotonin and norepinephrine.

The methods of the present invention are carried out, at least in part, using serotonin-norepinephrine reuptake inhibitors (SNRIs). SNRIs, e.g., venlafaxine (Effexor®) and duloxetine, generally function to correct the imbalance of both serotonin and norepinephrine in the brain. SNRIs have been used in the treatment of Major Depression and have also been found to be effective in several other disorders, including obsessive compulsive disorder, panic disorder, social phobia and in children with Attention Deficit Hyperactivity Disorder. Duloxetine has also been shown to relieve pain associated with depression (Goldstein, D L et al., 2004 Psychosomatics 45:1 7-28) Numerous studies have also implicated serotonin and norepinephrine systems in the neural control of lower urinary tract function. The lower urinary tract is innervated by parasympathetic, sympathetic and somatic divisions of the nervous system, as well as by primary afferent fibers, which function through central reflex mechanisms to allow storage and periodic elimination of urine. The parasympathetic, sympathetic and somatic spinal cord nuclei that control lower urinary tract function, and spinal areas that contain terminals of lower urinary tract primary afferent fibers, are densely innervated by serotonin and norepinephrine terminals. In addition, multiple subtypes of serotonin and norepinephrine receptors have been identified in each of the three efferent nuclei and in the spinal areas of primarily afferent terminations.

Pharmacological studies have shown that serotonin and norepinephrine receptor agonists and antagonists influence central control of lower urinary tract function. Furthermore, studies have shown a physiological role for endogenous serotonin and norepinephrine systems in lower urinary tract regulation.

Previous studies have shown that dual serotonin-norepinephrine reuptake inhibitors, e.g., duloxetine and venlafaxine, markedly increases bladder capacity and urethral sphincter electromyographic (EMG) activity in a cat model of acetic acid-induced bladder irritation, whereas serotonin selective reuptake inhibitors, e.g., S-norfluoxetine produced small increases in bladder capacity and EMG activity at doses of 3 and 10 mg/kg and NE selective reuptake inhibitors, e.g., thionisoxetine, produced no effects on bladder capacity or sphincter EMG activity. Additionally, co-administration of thionisoxetine and S-norfluoxetine up to doses of 1 mg/kg of each compound produced no effect on lower urinary tract function. These results indicate that there are unexplained pharmacological differences between the effects of single compounds that exhibit dual NE and 5-HT reuptake inhibition (i.e., an SNRI) and a combination of compounds that exhibit selective NE and 5-HT reuptake inhibition on lower urinary tract function.

Compounds with suitable SNRI activity can be identified by the skilled artisan using no more than routine experimentation (see, e.g., Wong, D. et al 1993, Neuropsychopharmacol. 8:23-33).

Glutamate Antagonists

The methods of present invention are also carried out using glutamate antagonists, e.g., NMDA antagonists. Excitatory amino acids are an important group of neurotransmitters that mediate excitatory neurotransmission in the central nervous system. Glutamic acid and aspartic acid are two endogenous ligands that activate excitatory amino acid (EAA) receptors. There are two types of EAA receptors, ionotropic and metabotropic, which differ in their mode of signal transduction. There are at least three distinct ionotropic EAA receptors characterized by the selective agonist that activate each type: the NMDA (N-methyl-D-aspartic acid), the AMPA (2-amino-3-(5-methyl-3-hydroxyisoxazol-4-yl) propanoic acid), and the kainic acid receptors.

The NMDA receptor is a macromolecular complex consisting of a number of distinct binding sites that gate an ion channel permeable to sodium and calcium ions. Hansen and Krogsgaard-Larsen, Med. Res. Rev., 10, 55-94 (1990). There are binding sites for glutamic acid, glycine, and polyamines, and a site inside the ion channel where compounds such as phencyclidine (PCP) and MK-801 exert their antagonist effects.

As used herein, the term “NMDA antagonist” refers to a functional NMDA antagonist. That is, an agent (e.g., a molecule, a compound, etc.) which interferes with the action of the NMDA receptor. NMDA antagonists, in some cases, may competitively, non-competitively or uncompetitively block endogenous compounds from binding to the glutamate binding site on the NMDA receptor. Alternatively, NMDA antagonists may inhibit binding to the strychnine-insensitive glycine site present on the NMDA receptor. As such, NMDA antagonist may be a non-competitive NMDA receptor antagonist, a competitive NMDA receptor antagonist, a glycine-site antagonist, a glutamate-site antagonist, an NR1 subunit antagonist, an antagonist of an NR₂ subunit, or an NR₃ subunit antagonist. The use of the term “antagonist” is in a functional sense and is not intended to limit the invention to compounds having a particular mechanism of action.

Antagonists of neurotransmission at NMDA receptors may prove to be useful therapeutic agents for the treatment of neurological disorders. U.S. Pat. No. 4,902,695 is directed to series of competitive NMDA antagonists useful for the treatment of neurological disorders, including epilepsy, stroke, anxiety, cerebral ischemia, muscular spasms, and neurodegenerative disorders such as Alzheimer's disease and Huntington s disease. U.S. Pat. No. 4,968,878 is directed to a second series of competitive NMDA receptor antagonists useful for the treatment of similar neurological disorders and neurodegenerative disorders.

Bladder activity is controlled by parasympathetic preganglionic neurons in the sacral spinal cord. de Groat et al., J. Auton. Nerv. Sys., 3, 135-160 (1981). In humans, it has been shown that the highest density of NMDA receptors in the spinal cord is located at the sacral level, including those areas that putatively contain bladder parasympathetic preganglionic neurons. Shaw et al., Brain Res., 539, 164-168 (1991). Because NMDA receptors are excitatory in nature, pharmacological blockade of these receptors would suppress bladder activity. In fact, Thor, U.S. Pat. No. 5,192,752 is directed to competitive NMDA antagonists for the treatment of urinary incontinence.

Compounds with suitable NMDA antagonist activity can be identified by the skilled artisan using no more than routine experimentation. For example, the ability of a particular compound to competitively bind to the NMDA glutamate receptor is determined using a radioligand binding assay. See Murphy et al., British J. Pharmacol., 95, 932-938 (1988). Other assays to determine the extent of NMDA antagonism can be found, for example, in Hemstapat, et al. J. Pharmacol. Toxicol. Methods, 49 : 81-87, (2004); Boje, et al., Brain Res., 603(2): 207-214 (1993); and Patel et al., J. Neurochem., 54: 849-854 (1990).

The present invention utilizes dual acting SNRI-NMDA antagonists, i.e., agents or groups of agents possessing both SNRI activity and NMDA antagonist activity. Without wishing to be bound by any particular theory, it is believed that the SNRI activity allows relaxation of the bladder and facilitation of the sphincter while the glutamate antagonist activity allows relaxation of the bladder and the sphincter, counteracting the effects of the SNRI on the sphincter. For example, it has been postulated that glutamate activation of sphincter motor neurons is necessary for SNRIs such as duloxetine to increase sphincter activity (Thor, K. B. 2004, Int. J. Obst. Gynecol., 38 (Suppl. 1:S38-S52). Furthermore, it is believed that the function of SNRIs to relax the bladder is not dependent on a direct glutamatergic link at the inhibitory site of serotonin or norepinephrine action. However, indirectly the SNRI is believed to be dependent upon a glutamatergic link because the release of serotonin and norepinephrine from axon terminals is influenced by the firing of action potentials by serotonin and norepinephrine neurons, which is influenced by glutamate receptors. Surprisingly, this indirect link does not necessarily prevent the overall inhibitory effect of the dual SNRI-NMDA receptor antagonist. Accordingly, it is believed that the two activities may work synergistically to relieve symptoms of genitourinary disorders.

Genitourinary Disorders

The phrase “genitourinary disorder” is art recognized and encompasses any infection, disease or other disorder that affects the normal function of the urinary and/or reproductive systems. Genitourinary disorders that may be treated with dual acting SNRI-NMDA antagonists of the present invention include, but are not limited to overactive bladder, overactive bladder with sphincter dysfunction, urinary incontinence, urge urinary incontinence, stress urinary incontinence, Fowler's Syndrome, outlet obstruction, outlet insufficiency, pelvic hypersensitivity, sphincteric spasticity, detrusor hyperreflexia (neurogenic bladder), detrusor instability, benign prostatic hyperplasia (BPH), urethral stricture disease, tumors, interstitial (cell) cystitis, chronic pelvic pain syndrome, prostatodynia, prostatis, vulvodynia, vulvar vestibulitis, urethritis, and/or orchidalgia. Exemplary genitourinary disorders include overactive bladder, overactive bladder with sphincter dysfunction, stress urinary incontinence, Fowler's Syndrome, chronic pelvic pain syndrome, prostatitis, prostatodynia, vulvodynia, vestibulitis, and/or benign prostatic hyperplasia. In some embodiments, GU disorders do not include pain.

The phrase “symptom(s) of a genitourinary disorder”, means any disease state or symptom which is generally associated with the genitourinary tract or a result of any of the genitourinary disorders discussed herein, including, but not limited to, urinary urgency, urinary frequency, altered bladder capacity, micturition threshold, unstable bladder contractions, low flow rates, difficulty in initiating urination, suprapubic pain, urethral hypermobility, intrinsic sphincteric deficiency, mixed incontinence, stress incontinence, pelvic pain, and other symptoms related to genitourinary disorders.

In some embodiments, the dual acting SNRI-NMDA antagonists of the present invention are useful for treating a genitourinary disorder which is characterized by bladder-sphincter dyssynergia. As used herein, the term “bladder-sphincter dyssynergia” refers to the partial or complete lack of cooperation between the bladder and the sphincter, which may be present in certain genitourinary disorders, e.g, Fowler's syndrome. Although the function is complex, it is believed that, in order for micturition to occur, the bladder must contract and the sphincter must relax concurrently. In certain genitourinary disorders, there is not only a malfunction of the bladder (e.g., an inability to relax, which may cause urinary frequency), but also a malfunction of the sphincter (e.g., an inability to relax, which may cause an outlet obstruction). As stated above, it is believed that the SNRI activity allows relaxation of the bladder and facilitation of the sphincter while the glutamate antagonist activity allows relaxation of the bladder and the sphincter, counteracting the effects of the SNRI on the sphincter.

In another embodiment, the genitourinary disorder is a genitourinary disorder with outlet obstruction. As used herein, the term “outlet obstruction” is used to describe any impediment, e.g., tumor or blockage, which at least partially prevents urination.

Normally, a coordinated activity between smooth muscle of the urinary bladder (e.g., the detrusor muscle of the urinary bladder) and striated muscle of the urethral sphincter controls micturition. The nerves that control these muscles allow a switching between storage and elimination of urine. Generally, the smooth muscle of the urinary bladder includes stretch receptors, which are responsible for the sensory reaction, or the need to urinate. For example, the bladder stretch receptors may be responsible for the urge that wakes one during nocturia. The striated muscle of the urethral sphincter, on the other hand, are generally responsible for involuntary loss of urine due to, e.g., coughing or sneezing. For example and in contrast to nocturia, control of the sphincter would be responsible for the loss of urine in nocturnal enuresis. Thus, control of the bladder stretch receptors is associated with urge urinary incontinence and control of the urthral sphincter is associated with stress urinary incontinence. In some embodiments, the dual acting SNRI-NMDA antagonists of the present invention are particularly useful for genitourinary disorders which are effected by the smooth muscle of the urinary bladder, e.g., urge urinary incontinence.

As used herein, “overactive bladder” and “OAB” are used interchangeably and refer to a chronic condition resulting from overactivity of the detrusor muscle, wherein the bladder initiates contraction too early while filling with urine, manifesting with one or more symptoms of urinary frequency, urinary urgency, urinary urge incontinence, nocturia or enuresis. Loss of voluntary control may generally range form partial to total, and may or may not include a loss of urine (incontinence). Overactive bladder may manifest itself as wet OAB, that is overactive bladder in patients with incontinence, or dry OAB, that is overactive bladder in patients without incontinence. Overactive bladder can be neurogenic or non-neurogenic.

Neurogenic overactive bladder (or neurogenic bladder) is a type of overactive bladder which occurs as a result of detrusor muscle overactivity referred to as detrusor hyperreflexia, secondary to neurologic disorders.

Non-neurogenic overactive bladder occurs as a result of detrusor muscle overactivity referred to as detrusor muscle instability. Detrusor muscle instability can arise from non-neurological abnormalities, such as bladder stones, muscle disease, urinary tract infection or drug side effects or can be idiopathic.

Accordingly, in some embodiments, the compounds and/or compositions of the present invention may be administered to decrease overactivity or hyperactivity of the detrusor muscle. In some embodiments, a dual acting SNRI-NMDA antagonist may be used to treat detrusor hyperreflexia. In other embodiments, a dual acting SNRI-NMDA antagonist may be used to treat detrusor muscle instability.

As used herein, “bladder disorder” refers to any condition involving the urinary bladder.

As used herein, the term “Fowler's Syndrome” is a bladder emptying disorder, typically in women between the ages of 18 and 35 years with no other neurological symptoms, that shows complex repetitive discharges and decelerations on concentric needle EMG of the external urethral sphincter which prevent its relaxation and cause urinary retention.

“Interstitial cystitis” is used herein in its conventional sense to refer to a disorder associated with symptoms that can include irritative voiding symptoms, urinary frequency, urgency, nocturia, suprapubic pain and/or pelvic pain related to and relieved by voiding.

“Vulvodynia” refers to vulvar vestibulitis syndrome which is elicited by touch of the vestibule area and dysesthetic vulvodynia which is not limited to the vestibule and may occur without touch or pressure.

As used herein, “urinary frequency” refers to urinating more frequently than the patient desires. As there is considerable interpersonal variation in the number of times in a day that an individual would normally expect to urinate, “more frequently than the patient desires” is further defined as a greater number of times per day than that patient's historical baseline. “Historical baseline” is further defined as the median number of times the patient urinated per day during a normal or desirable time period.

As used herein, “urinary urgency” refers to sudden strong urges to urinate with little or no chance to postpone the urination.

As used herein, “urinary urge incontinence” (also referred to as urge incontinence) refers to the involuntary loss of urine associated with urinary urgency.

As used herein, the terms “stress incontinence,” “urinary stress incontinence,” and “stress urinary incontinence” are used interchangeably to refer to a medical condition in which urine leaks when a person coughs, sneezes, laughs, exercises, lifts heavy objects, or does anything that puts pressure on the bladder.

As used herein, “nocturia” refers to being awakened from sleep due to the urge to urinate more frequently than the patient desires.

As used herein, “enuresis” refers to involuntary voiding of urine which can be complete or incomplete. Nocturnal enuresis refers to enuresis which occurs during sleep. Diurnal enuresis refers to enuresis which occurs while awake.

As used herein, “prostatitis” refers to any type of disorder associated with inflammation of the prostate, including chronic and acute bacterial prostatitis and chronic non-bacterial prostatitis, and which is usually associated with symptoms of urinary frequency and/or urinary urgency.

Acute and chronic bacterial prostatitis are used herein in the conventional sense to refer to a disorder characterized by inflammation of the prostate and bacterial infection of the prostate gland, usually associated with symptoms of pain, urinary frequency and/or urinary urgency. Chronic bacterial prostatitis is distinguished from acute bacterial prostatitis based on the recurrent nature of the disorder. Chronic non-bacterial prostatitis is used herein in its conventional sense to refer to a disorder characterized by inflammation of the prostate which is of unknown etiology accompanied by the presence of an excessive amount of inflammatory cells in prostatic secretions not currently associated with bacterial infection of the prostate gland, and usually associated with symptoms of pain, urinary frequency and/or urinary urgency.

Prostatodynia is a disorder which mimics the symptoms of prostatitis absent inflammation of the prostate, bacterial infection of the prostate and elevated levels inflammatory cells in prostatic secretions. Prostatodynia can be associated with symptoms of pain, urinary frequency and/or urinary urgency.

Benign prostatic hyperplasia is used herein in its conventional sense to refer to a disorder associated with benign enlargement of the prostate gland which can be associated with urinary frequency, urinary urgency, urge incontinence, nocturia, and/or reduced urinary force and speed of flow.

The invention relates to a method of treating a genitourinary disorder in a subject in need of treatment. The method comprises administering to the subject in need of treatment a therapeutically effective amount of a dual acting SNRI-NMDA antagonist, such that the genitourinary disorder is treated. The dual acting SNRI-NMDA antagonist may include a first amount of an SNRI and a second amount of an NMDA antagonist. The first amount and the second amount can both be a therapeutically effective amount. Alternatively, the first amount and the second amount together form a therapeutically effective amount. The dual acting SNRI-NMDA antagonist may optionally include one agent having both SNRI activity and NMDA antagonist activity.

In a particular embodiment, the dual acting SNRI-NMDA antagonists are 1-phenyl-3-azabicyclo[3.1.0]hexane derivatives such as those described in U.S. Pat. Nos. 4,131,611, 4,196,120, 4,231,955, and 4,435,419 the entire contents of which are incorporated herein by reference.

In specific embodiments, the dual acting SNRI-NMDA antagonist is a compound of Formula I:

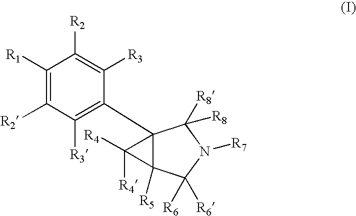

wherein R₁, R₂, R₂′, R₃, R₃′, R₄, R₄′, R₅, R₆, R₆′, R₇, R₈, and R₈′ are each independently H, alkyl, aryl, alkenyl, alkynyl, alkoxy, alkoxyalkyl, cycloalkyl, acyl, aroyl, carboxyl, carbonyl, amino, alkylamino, dialkylamino, nitro, halogen, hydroxyl, amido, acetamido, or trifluoromethyl; optionally R₄ and R₄′ together form ═O, ═S, ═NH or ═CH₂; optionally R₆ and R₆═ together form ═O, ═S, ═NH or ═CH₂; and optionally R₈ and R₈′ together form ═O, ═S, ═NH or ═CH₂ or pharmaceutically acceptable salts thereof.

In some embodiments, R₁, R₂, R₂′, R₃ and R₃′ are each independently H, alkyl, aryl, alkoxy, alkoxyalkyl, cycloalkyl, amino, nitro, acetamido, hydroxyl, trifluoromethyl or halogen, R₄, R₄′, R₅, R₆, R₆′, R₈, and R₈′ are each independently H, alkyl, aryl, alkoxy, alkoxyalkyl, cycloalkyl, halogen or hydroxyl, optionally R₆ and R₆′ together form ═O, ═S, ═NH or ═CH₂, optionally R₈ and R₈′ together form ═O, ═S, ═NH or ═CH₂, and R₇ is H, alkyl, aryl, alkoxy, alkoxyalkyl, cycloalkyl, acyl, carboxyl or carbonyl.

In other embodiments, R₁, R₂, R₂′, R₃ and R₃′ are each independently H, C₁-C₆ alkyl, C₁-C₆ alkoxy or halogen; R₄, R₄′, R₅, R₆, R₆′, R₈, and R₈′ are each independently H, C₁-C₆ alkyl, halogen or hydroxyl; optionally R₆ and R₆′ together form ═O; and optionally R₈ and R₈′ together form ═O; and R₇ is H or C₁-C₆ alkyl optionally substituted with aryl or substituted aryl.

In some embodiments, at least one of R₁, R₂, R₂′, R₃ and R₃′ is other than hydrogen.

In some embodiments, R₁ and R₂′ are not both chlorine if R₂, R₃, R₃′, R₄, R₄′, R₅, R₆, R₆′, R₇, R₈, and R₈′ are hydrogen.

In other embodiments, the compound represented by formula I is a single enantiomer.

In another embodiment, the dual acting SNRI-NMDA antagonist is represented by one of the following structural formulas:

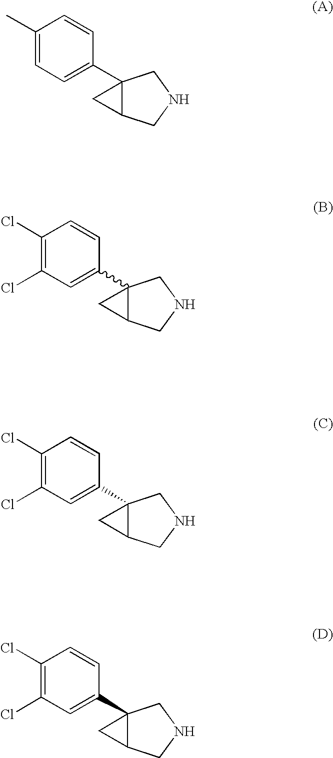

In another particular embodiment, the dual acting SNRI-NMDA antagonists are 2-(aminomethyl)-1-phenylcyclopropanecarboxamide derivatives, such as those described in U.S. Pat. Nos. 3,989,722, 4,478,836, 5,621,142, 6,602,911 and 6,635,675.

In another specific embodiment, the compounds having SNRI activity and NMDA antagonist activity are represented by Formula II:

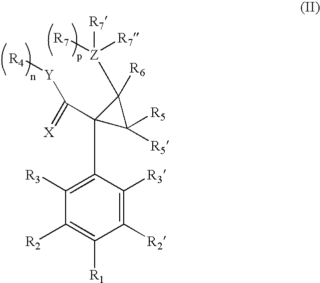

wherein X is selected from O, S and NR; Y is selected from O, S, and NR₄′; n and p are each independently 0 or 1; optionally R₇ and R₄ together form a direct bond between Y and Z; R, R₁, R₂, R₂′, R₃, R₃′, R₅, R₅′, R₆, R₇, and R₇′ are each independently H, alkyl, aryl, heteroaryl, arylalkyl, alkenyl, alkynyl, alkoxy, alkoxyalkyl, aryloxy, arylalkyloxy, cycloalkyl, acyl, aroyl, carboxyl, carbonyl, amino, alkylamino, dialkylamino, arylamino, arylakylamino, nitro, halogen, hydroxyl, amido, acetamido, or trifluoromethyl, cyano, thio, alkylthio, arylthio, arylakylthio, azido, alkylseleno, formyl, silyl, silyloxy, (alkyloxy)carbonyl, (aryloxy)carbonyl, (arylalkyloxy)carbonyl, (alkylamino)carbonyl, (arylamino)carbonyl, (arylalkylamino) carbonyl, alkylsulfonyl, arylsulfonyl, or —(CH₂)ₘ—R₉; R₇″ is selected from H, NR₈R₈′, OR₈, and SR₈; R₉ is selected from aryl, cycloalkyl, heterocyclyl or polycyclyl; m is an integer from 0 to 8; R₄, R₄′, R₈, and R₈′ are each independently H, alkyl, aryl, aralkyl, alkenyl, alkynyl, alkoxyalkyl, cycloalkyl, acyl, aroyl, carboxyl, carbonyl; optionally R₄ and R₄′ together form a heterocyclic ring; optionally R₈ and R₈′ together form a heterocyclic ring; and optionally R₇ and R₇′ together form ═O, ═S, ═NH or ═CH₂; or pharmaceutically acceptable salts thereof.

In some embodiments, X is O; Y is O or NR₄′; n and p are each 1; R₁, R₂, R₂′, R₃, R₃′, are each independently H, alkyl, alkoxy, amino, nitro, halogen, or hydroxyl; R₄ and R₄′ are each independently H, alkyl, aryl, aralkyl, or optionally R₄ and R₄′ together form a heterocyclic ring; R₇″ is NR₈R₈′ or OR₈; R₈, and R₈′ are each independently H, alkyl, or optionally R₈ and R₈′ together form a heterocyclic ring; and R₅, R₅′, R₆, R₇, and R₇′ are each independently H, alkyl, or aryl. In some embodiments, n and p are each independently 0 and R₄ and R₇ together form a direct bond between Y and Z.

In other embodiments, R₁, R₂, R₂′, R₃ and R₃′ are H; X is O; Y is O or NR₄′; n and p are each 1; R₇″ is H, NR₈R₈′ or OR₈; R₄, R₄′, R₅, R₅′, R₆, R₇, R₇′ are independently H or C₁-C₆ alkyl. In still other embodiments, R₁, R₂, R₂′, R₃ and R₃′ are H; X is O; Y is O or NR₄′; n and p are each 0 and R₄ and R₇ together form a direct bond between Y and Z; R₇″ is H, NR₈R₈′ or OR₈; R₄′, R₅, R₅′, R₆, R₇′, R₈, and R₈′ are each independently H or C₁-C₆ alkyl.

In one embodiment, R₄ and R₄′ are each ethyl.

In other embodiments, the compound represented by formula II is a single enantiomer. In still other embodiments, the compound represented by formula II is not milnacipran.

In another embodiment, the the dual acting SNRI-NMDA antagonist is represented by the following structural formula:

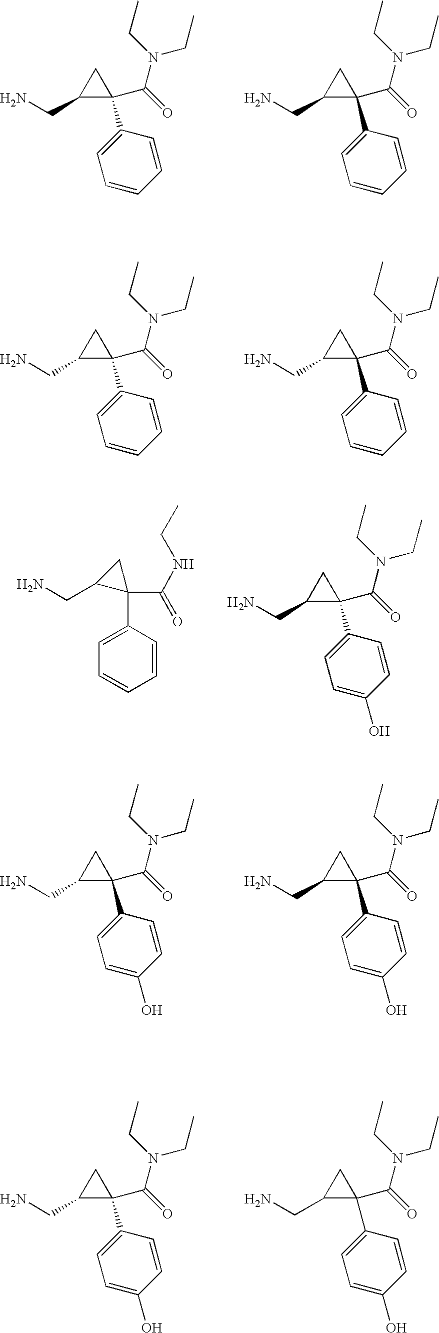

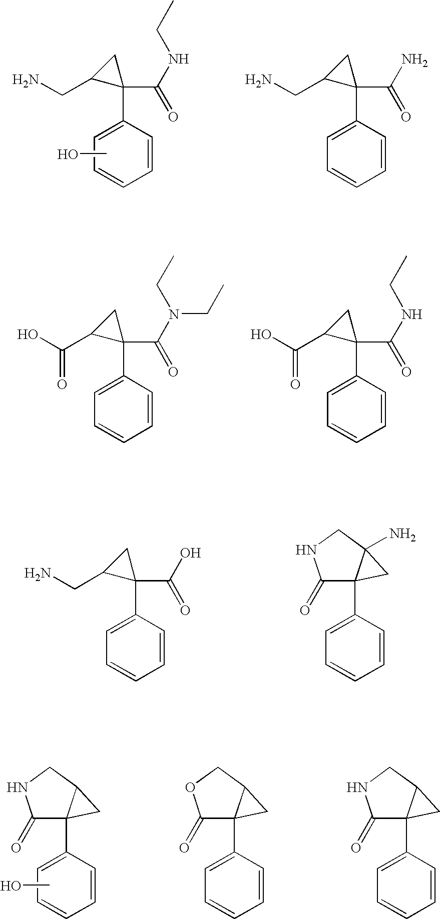

In still another particular embodiment, the dual acting SNRI-NMDA antagonists are diarylalkanamines and derivatives, such as those described in, for example, U.S. Pat. Nos. 3,510,560, 6,017,965, 6,071,970, and 6,211,245, and International Patent Application Numbers WO00/02551, WO98/56752, WO97/46511 and WO96/40097.

In still other specific embodiments, the dual acting SNRI-NMDA antagonist is a compound of Formula III:

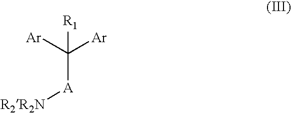

wherein each Ar is independently cycloalkyl, aryl, aralkyl, heteroaryl or heteroaralkyl group optionally substituted with one or more amino, alkylamino, dialkylamino, alkyl, hydroxyl, alkoxy, mercapto, alkylthio, alkylsulfinyl, acyl, halogen, perhaloalkyl, trifluoromethyl, trifluoromethylthio, trifluoromethylsulfonyl, and/or trifluoromethoxy; optionally each Ar is taken together to form a fused polycyclic ring system; R₁ is H, alkyl, aryl or aralkyl; R₂ and R₂′ are each independently H, alkyl, alkylaryl, acyl, or R₂ and/or R₂′ are taken together with A to form a heterocycle or a heteroaryl; and A is alkylene, alkenylene or alkynylene, optionally interrupted with —O—, —S—, or —NH—, or pharmaceutically acceptable sats thereof.

In some embodiments, each Ar is independently phenyl, phenoxy, benzyl, naphthyl, thiofuranyl, tetrahydronaphthyl, pyridyl, quinolinyl, isoquinolinyl, tetrahydroquinolinyl, tetrahydroisoquinolinyl, cyclohexyl, cycloheptyl or cyclopentyl, optionally substituted with one or more amino, alkylamino, dialkylamino, alkyl, hydroxyl, alkoxy, mercapto, alkylthio, alkylsulfinyl, acyl, halogen, perhaloalkyl, trifluoromethyl, trifluoromethylthio, trifluoromethylsulfonyl, and/or trifluoromethoxy; optionally each Ar is taken together to form a dibenzo[7]annulene, a dihydrodibenzo[7]annulene, a xanthenyl or a thioxanthenyl; R₁ is H or alkyl; R₂ and R₂′ are each independently H, alkyl or R₂ and/or R₂′ are taken together with A to form a heterocycle; and A is a liner or branched alkylene or alkenylene, optionally interrupted with an —O—.

In other embodiments, the compound represented by formula I is a single enantiomer.

In another embodiment, the dual acting SNRI-NMDA antagonist is represented by one of the following structural formulas:

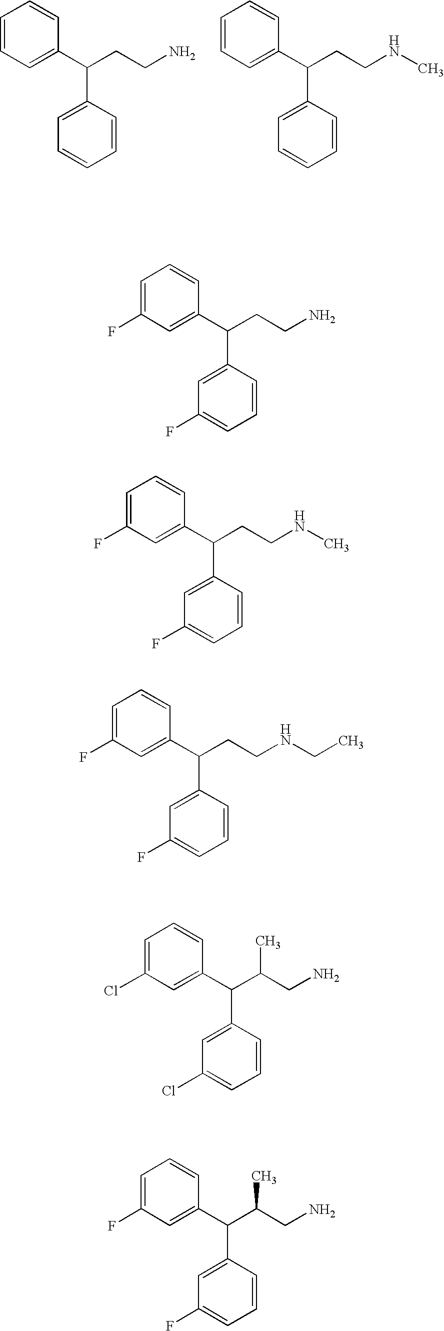

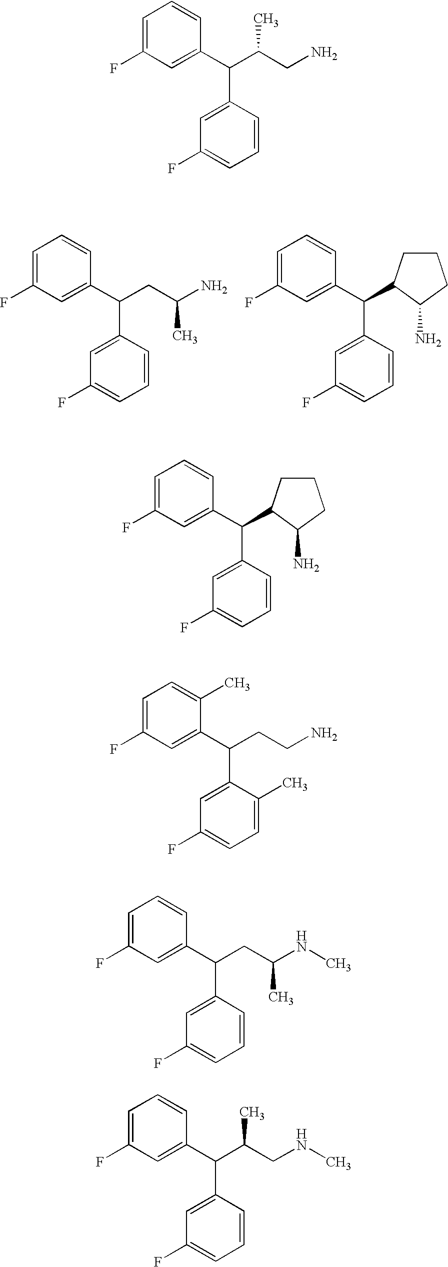

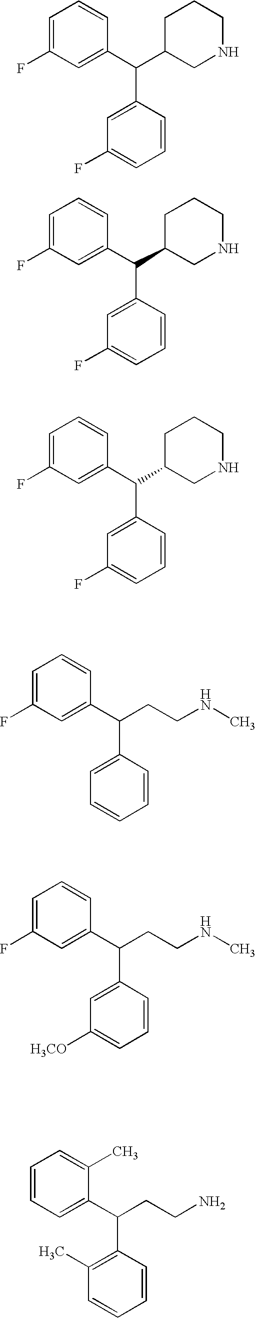

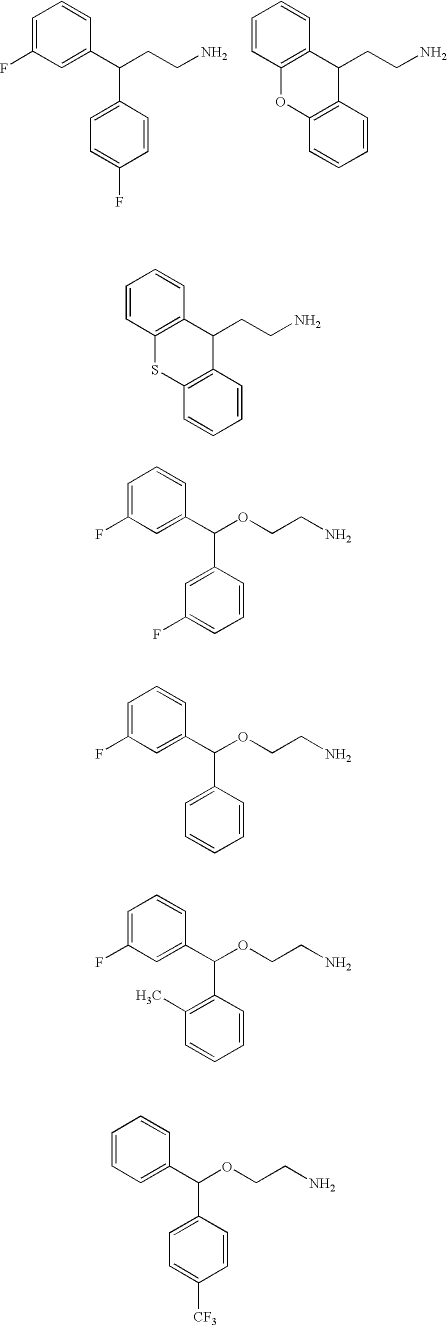

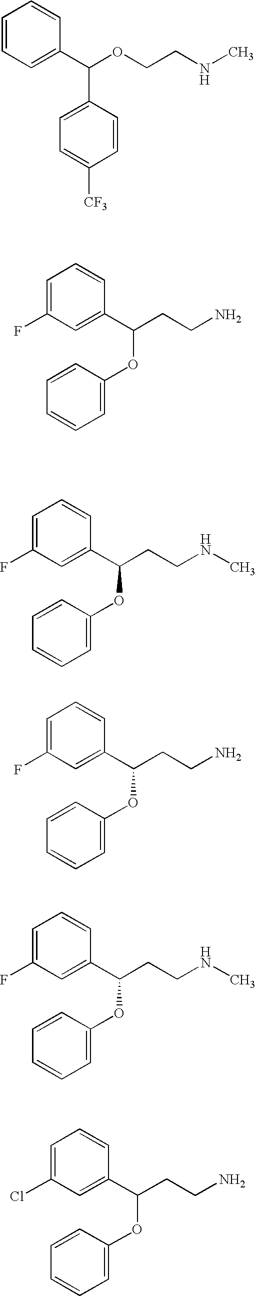

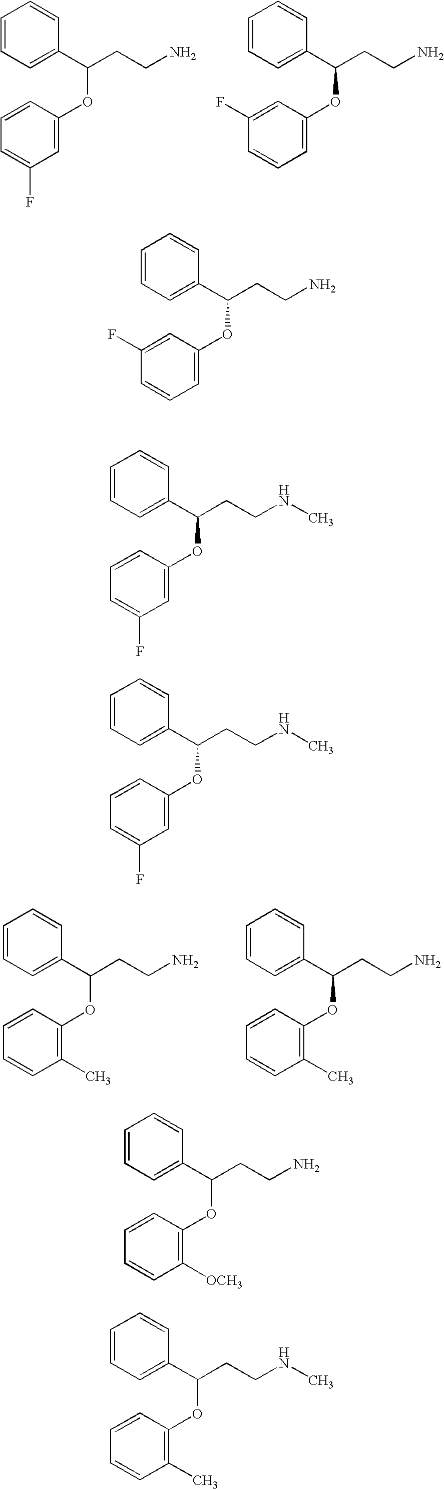

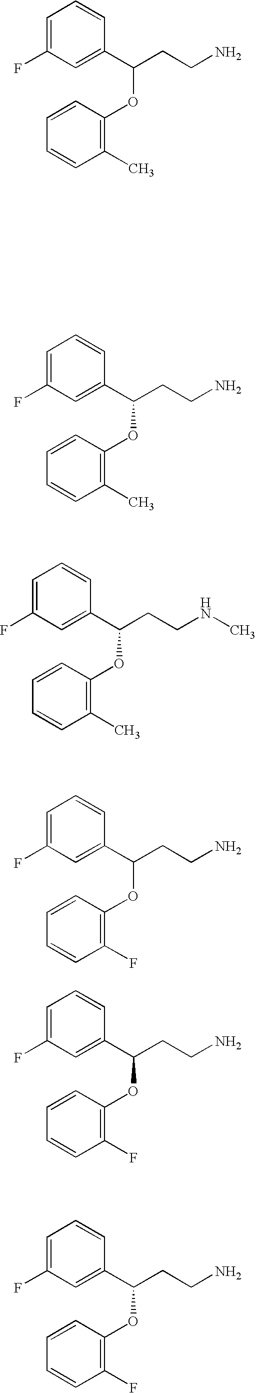

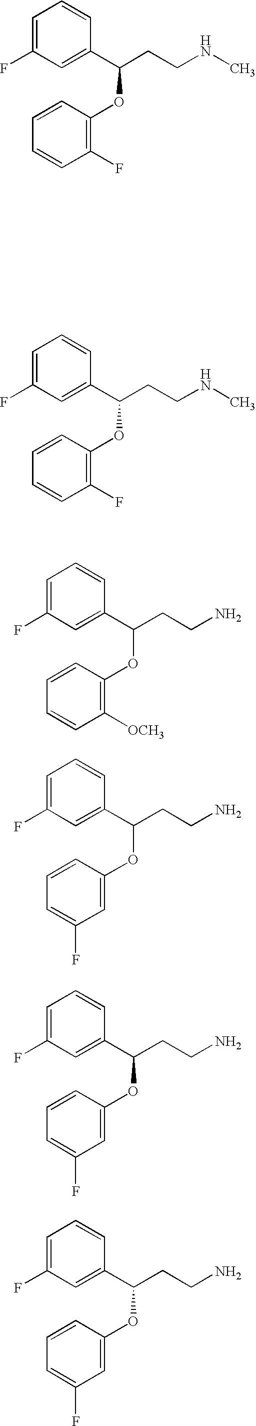

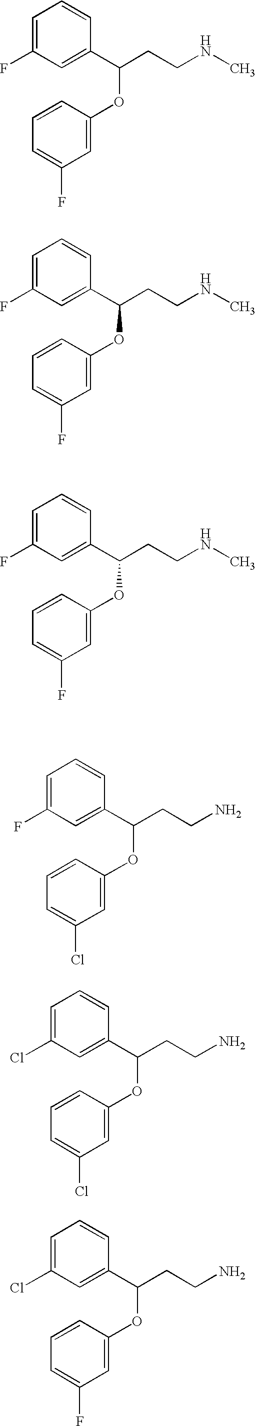

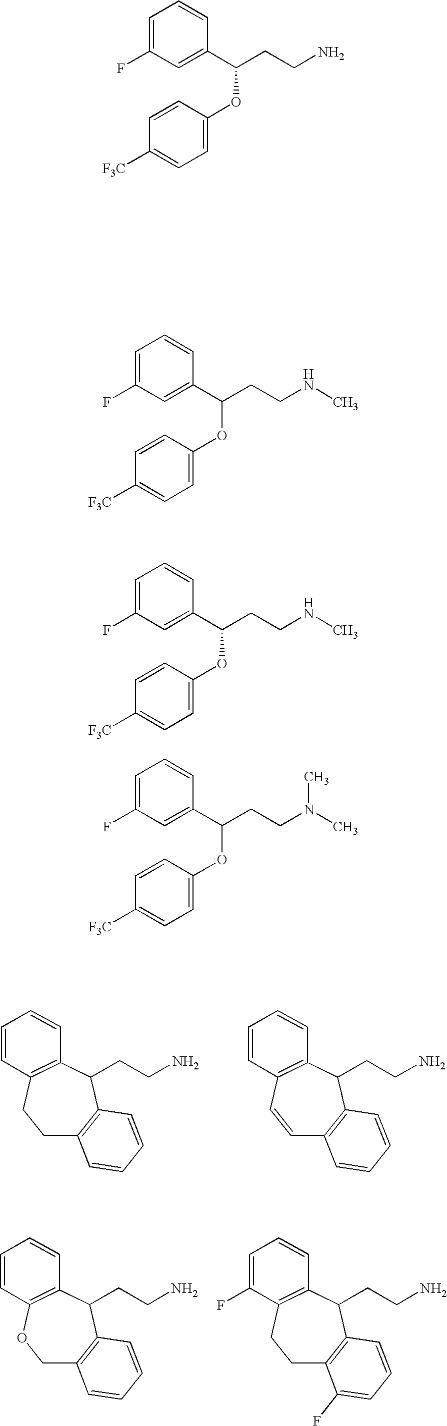

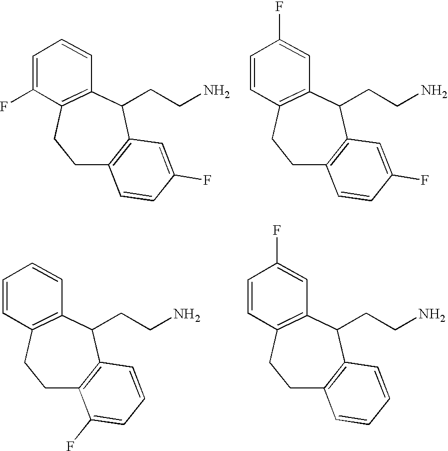

In another embodiment, the dual acting SNRI-NMDA antagonist is represented by one of the following structural formulas:

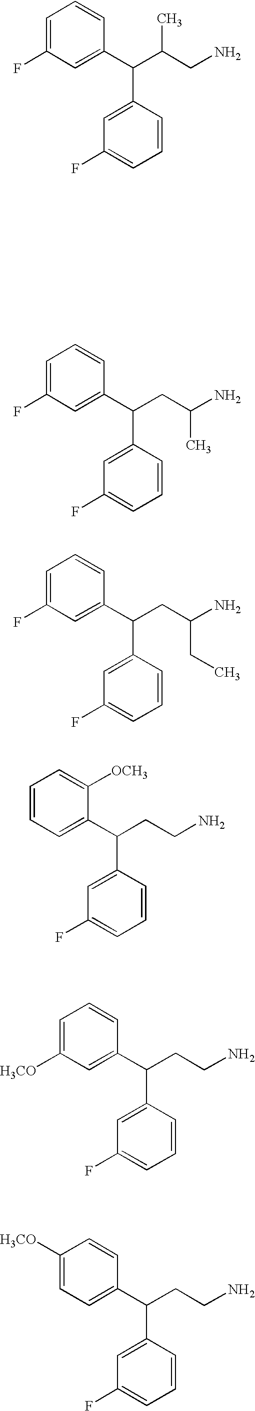

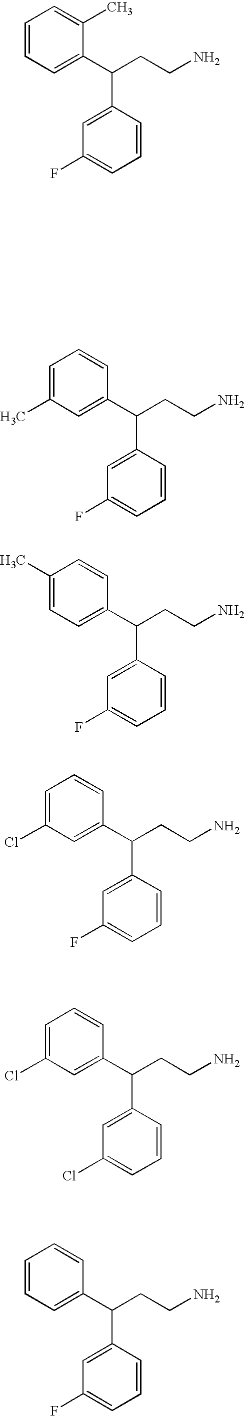

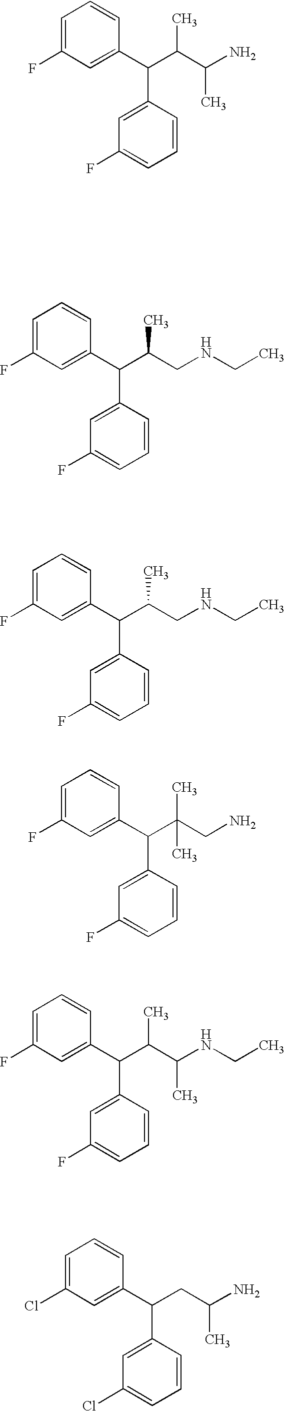

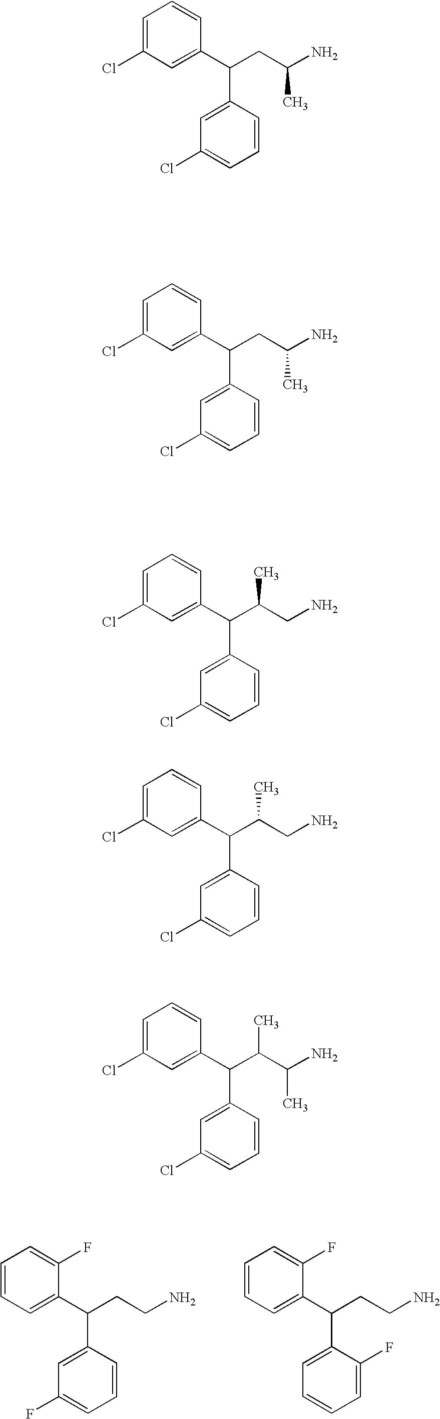

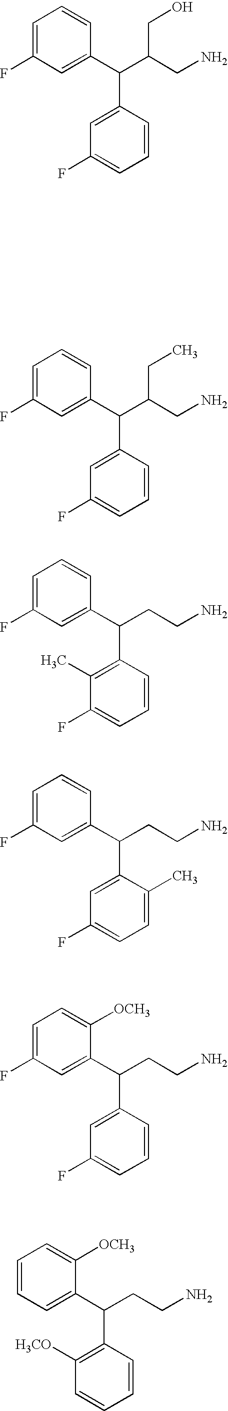

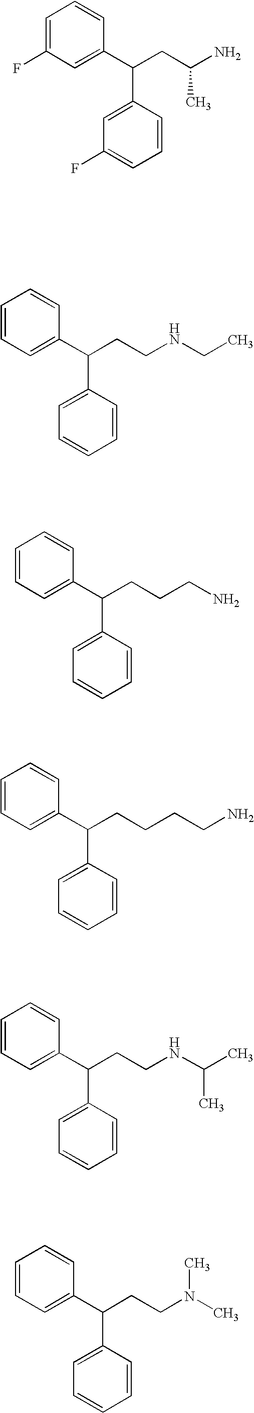

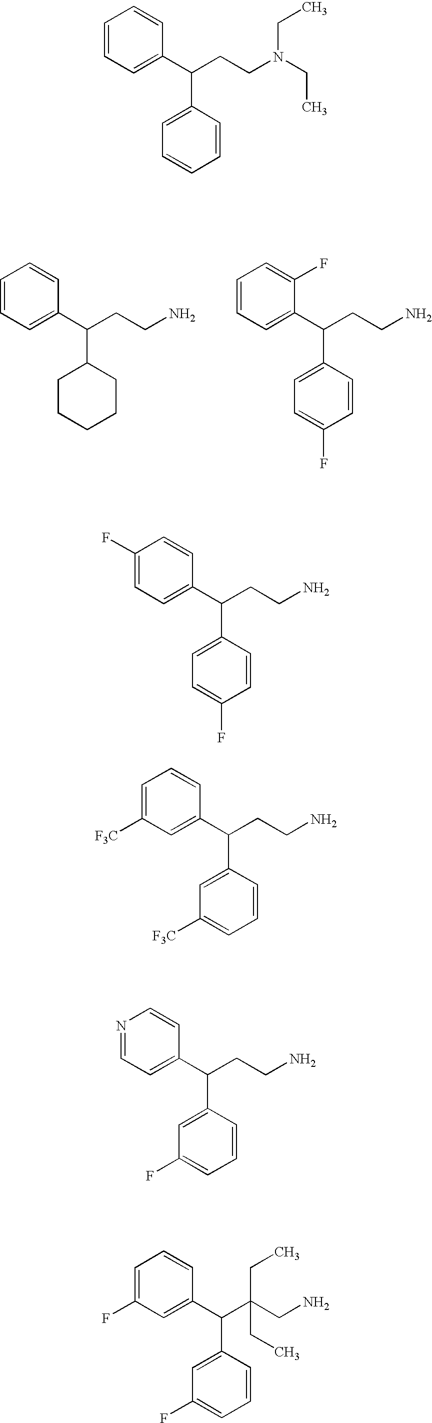

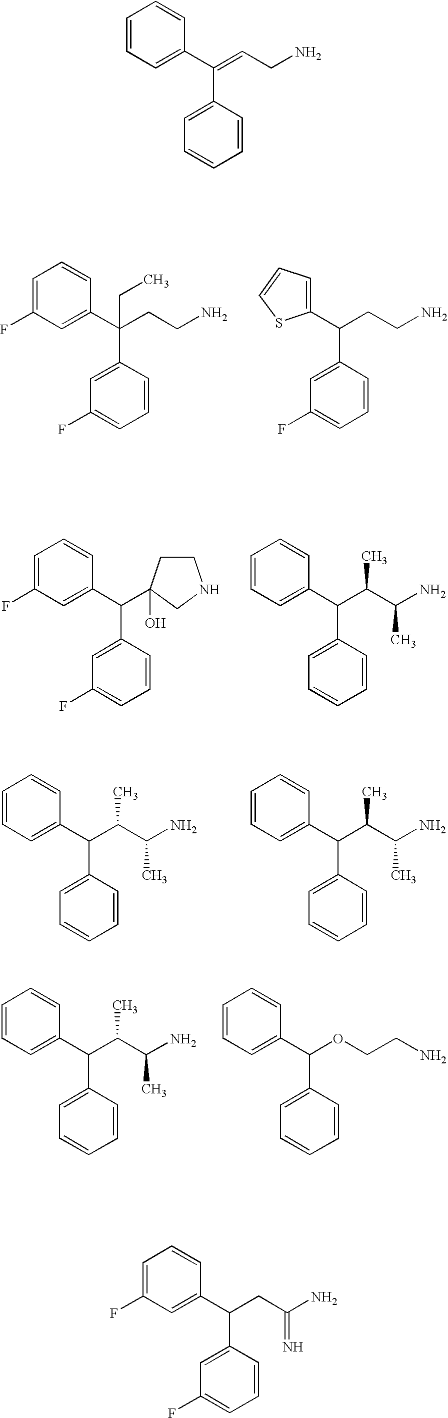

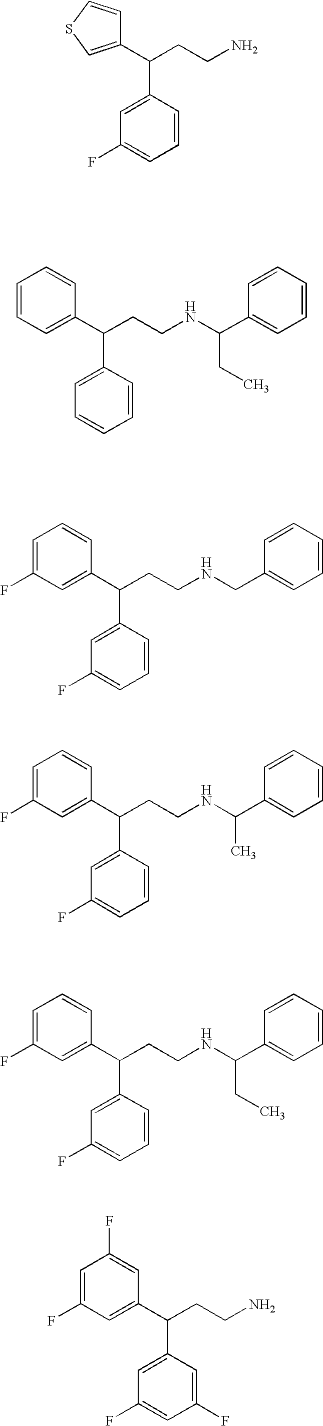

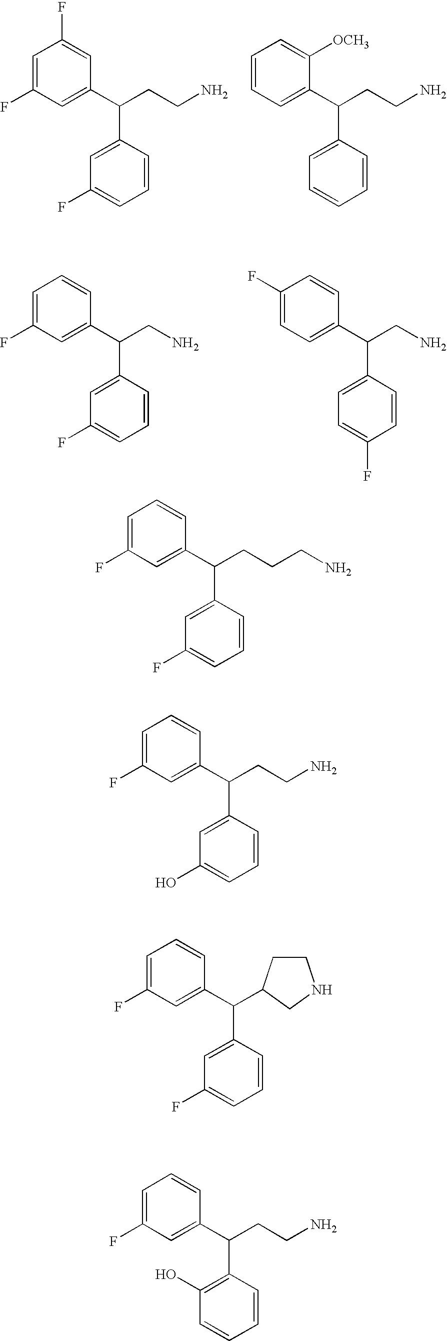

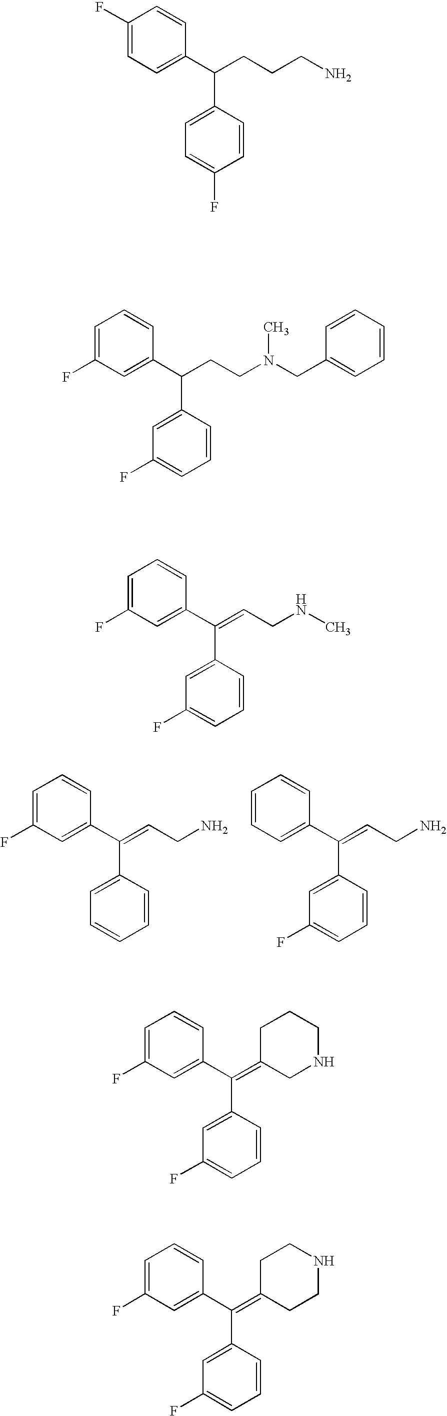

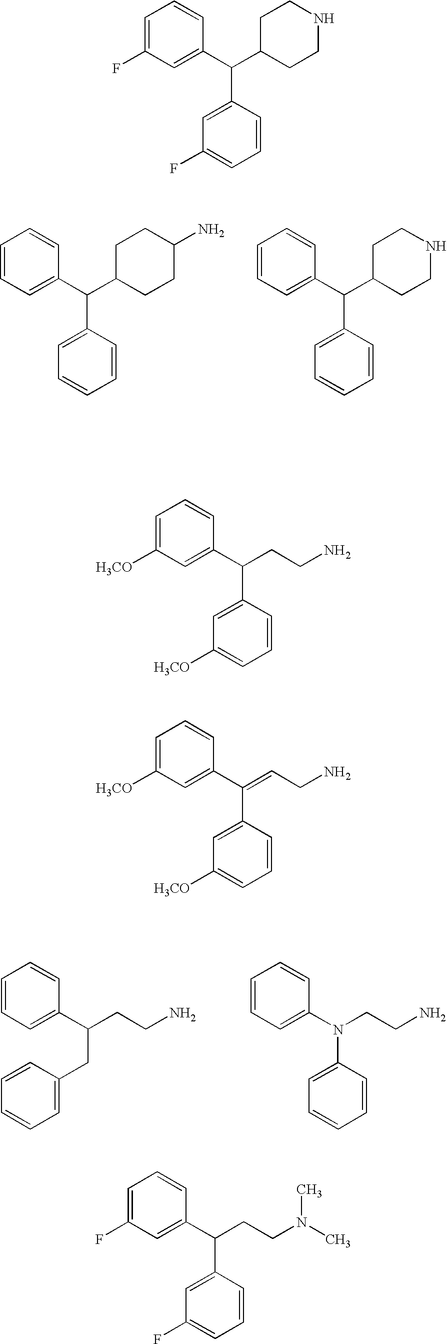

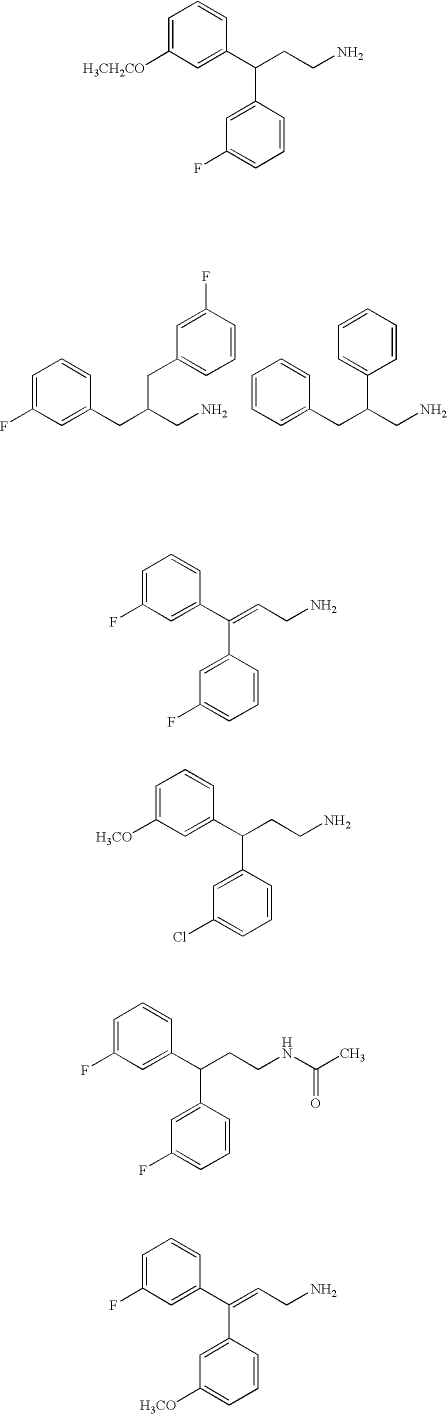

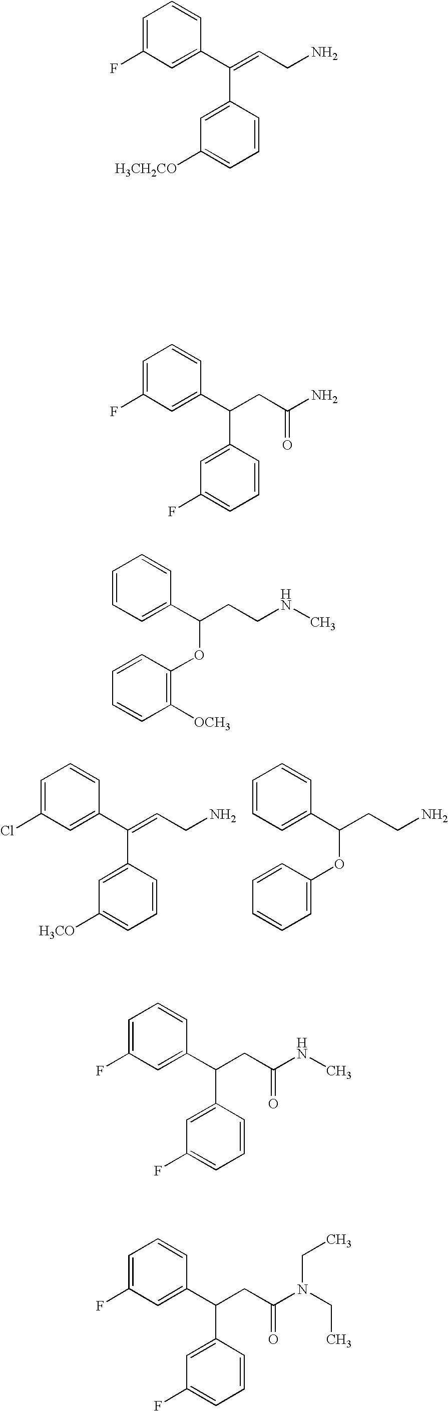

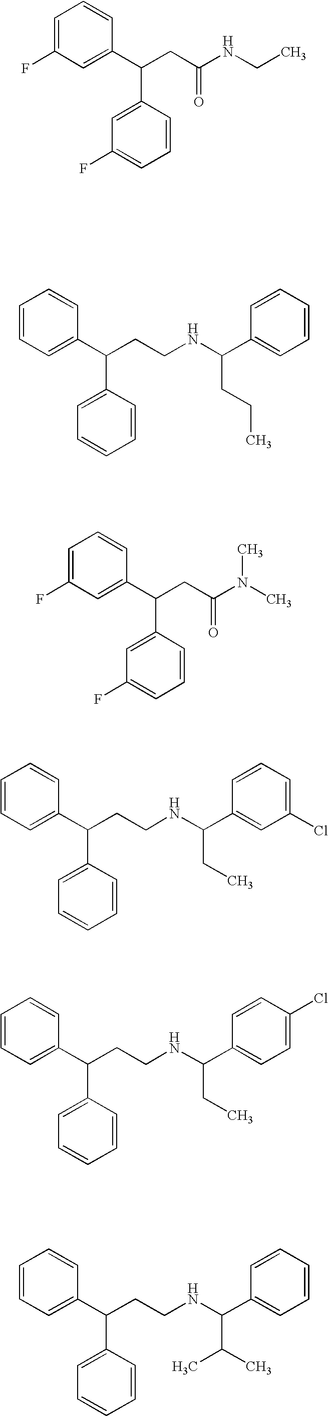

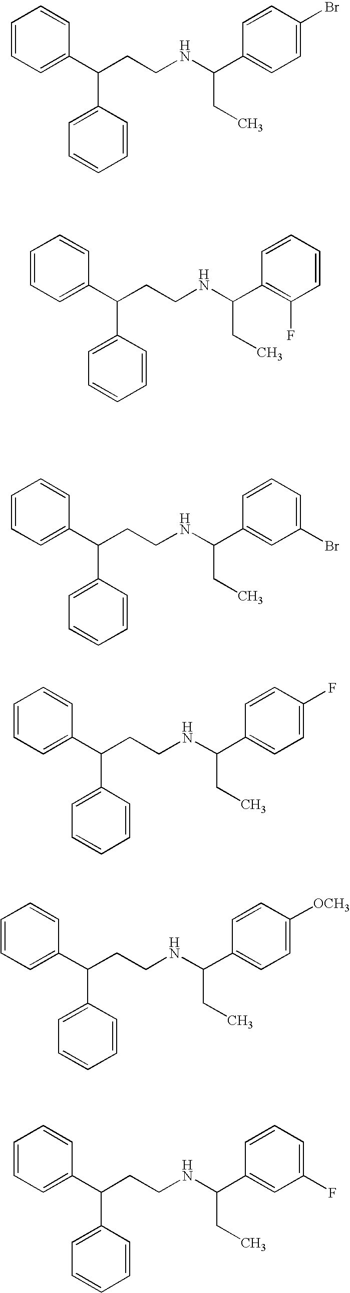

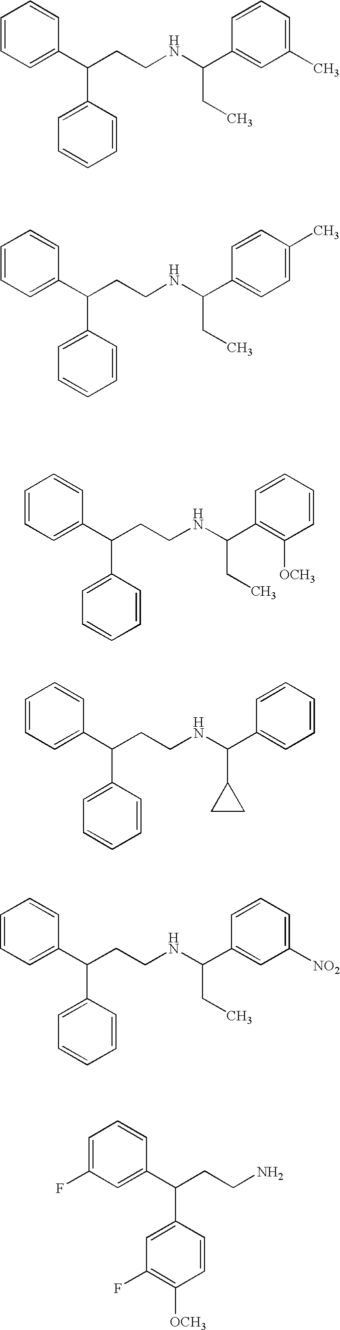

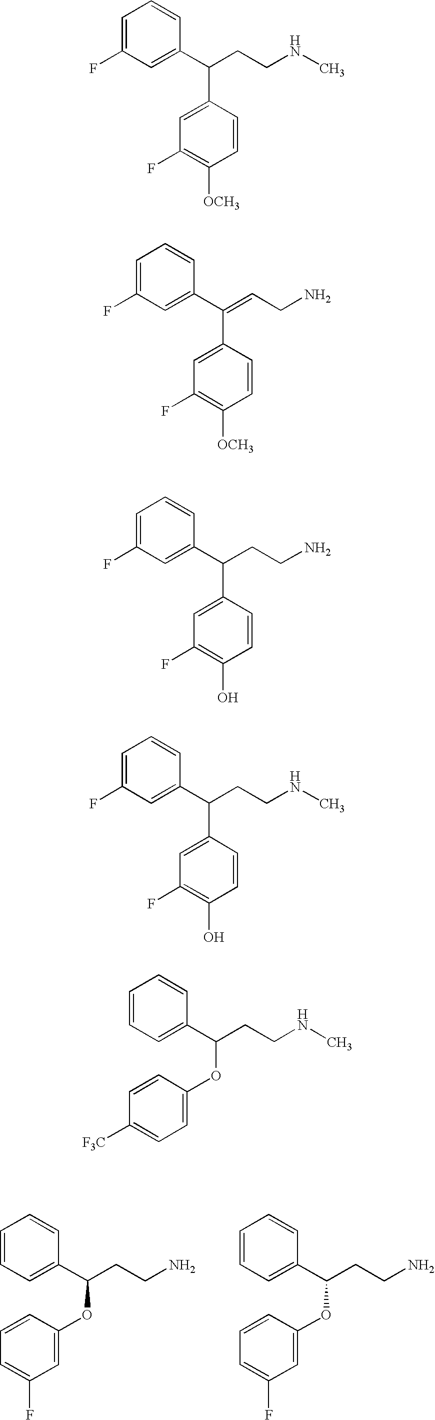

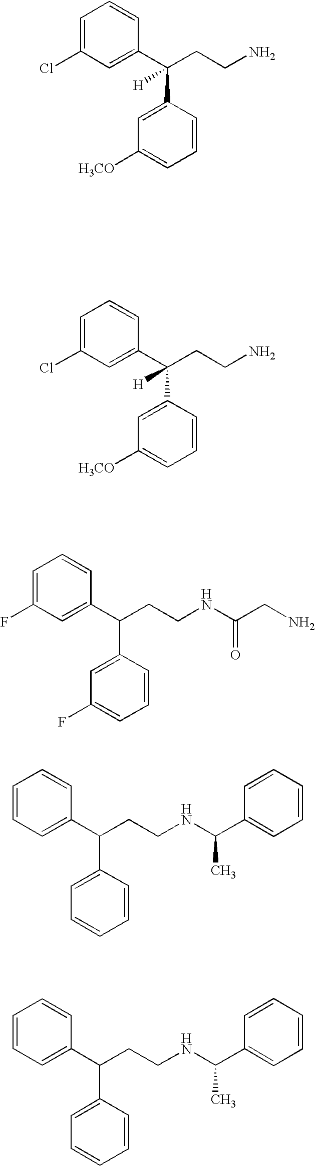

In some embodiments, the present invention includes the treatment of a genitourinary disorder using bicifadine compounds, milnacipran compounds and/or delucemine compounds.

Bicifadine compounds which are particularly effective for this purpose include bicifadine, 1-(3,4-Dichlorophenyl)-3-azabicyclo[3.1.0]hexane, and analogs thereof which are described in detail below. The term “bicifadine compounds” will be used herein to include bicifadine, 1-(3,4-Dichlorophenyl)-3-azabicyclo[3.1.0]hexane, and compounds which are structurally similar to bicifadine or 1-(3,4-Dichlorophenyl)-3-azabicyclo[3.1.0]hexane, analogs of bicifadine and 1-(3,4-Dichlorophenyl)-3-azabicyclo[3.1.0]hexane, and combinations thereof. The term “bicifadine compounds” also includes compounds which “mimic” the activity of bicifadine, 1-(3,4-Dichlorophenyl)-3-azabicyclo[3.1.0]hexane or bicifadine analogs, i.e., compounds or groups of compounds having dual SNRI-NMDA antagonist activity. The term bicifadine compound is also intended to include pharmaceutically acceptable or physiologically acceptable salts of the compounds. Particularly preferred are bicifadine compounds which exhibit dual SNRI-NMDA antagonist activity.

Milnacipran compounds which are particularly effective for this purpose include milnacipran and analogs thereof which are described in detail below. The term “milnacipran compounds” will be used herein to include milnacipran and compounds which are structurally similar to milnacipran, analogs of milnacipran, and combinations thereof. The term “milnacipran compounds” also includes compounds which “mimic” the activity of milnacipran or milnacipran analogs, i.e., compounds or groups of compounds having dual SNRI-NMDA antagonist activity. The term milnacipran compound is also intended to include pharmaceutically acceptable or physiologically acceptable salts of the compounds. Particularly preferred are milnacipran compounds which exhibit dual SNRI-NMDA antagonist activity. In some embodiments, the dual acting SNRI-NMDA antagonist is not milnacipran.

Delucemine compounds which are particularly effective for this purpose include delucemine, and analogs thereof which are described in detail below. The term “delucemine compounds” will be used herein to include delucemine and compounds which are structurally similar to delucemine, analogs of delucemine, and combinations thereof. The term “delucemine compounds” also includes compounds which “mimic” the activity of delucemine or delucemine analogs, i.e., compounds or groups of compounds having dual SNRI-NMDA antagonist activity. The term delucemine compound is also intended to include pharmaceutically acceptable or physiologically acceptable salts of the compounds. Particularly preferred are delucemine compounds which exhibit dual SNRI-NMDA antagonist activity.

Chemistry Terminology

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer (e.g. C₁-C₆). Likewise, preferred cycloalkyls have from 3-7 carbon atoms in their ring structure, and more preferably have 5 or 6 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylaamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety, which can optionally be substituted. Alkyls can also be perhalogenated. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids. Examples of halogenated alkyl groups include fluoromethyl, difluoromethyl, trifluoromethyl, chloromethyl, dichloromethyl, trichloromethyl, perfluoromethyl, perchloromethyl, perfluoroethyl, perchloroethyl, etc.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatic”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulflhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkylene” includes linear or branched carbon chains generally having from one to 10 carbons. The term “alkenylene” includes linear or branched carbon chains generally having from one to 10 carbons which include one or more double bonds. The term “alkynylene” includes linear or branched carbon chains generally having from one to 10 carbons which include one or more triple bonds.

Moreover, the terms alkylene, alkenylene, and alkynylene include both “unsubstituted alkylenes” and “substituted alkylenes”, “unsubstituted alkenylenes” and “substituted alkenylenes”, and “unsubstituted alkynylenes” and “substituted alkynylenes”, the latter of which refers to alkylene, alkenylene, and alkynylene moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide” or “aminocarboxy” includes compounds or moieties which contain a nitrogen atom which is bound to the carbon of a carbonyl or a thiocarbonyl group. The term includes “alkaminocarboxy” groups which include alkyl, alkenyl, or alkynyl groups bound to an amino group bound to a carboxy group. It includes arylaminocarboxy groups which include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties which contain a carbon connected with a double bond to an oxygen atom, and tautomeric forms thereof. Examples of moieties which contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “hydroxy” or “hydroxyl” includes groups with an −OH or —O—.

The term “halogen” includes fluorine, bromine, chlorine, iodine, etc. The term “perhalogenated” generally refers to a moiety wherein all hydrogens are replaced by halogen atoms.

The term “heteroatom” includes atoms of any element other than carbon or hydrogen. Preferred heteroatoms are nitrogen, oxygen, sulfur and phosphorus.

The term “heterocycle” or “heterocyclic” includes saturated, unsaturated, aromatic (“heteroaryls” or “heteroaromatic”) and polycyclic rings which contain one or more heteroatoms. Examples of heterocycles include, for example, benzodioxazole, benzofuran, benzoimidazole, benzothiazole, benzothiophene, benzoxazole, deazapurine, furan, indole, indolizine, imidazole, isooxazole, isoquinoline, isothiaozole, methylenedioxyphenyl, napthridine, oxazole, purine, pyrazine, pyrazole, pyridazine, pyridine, pyrimidine, pyrrole, quinoline, tetrazole, thiazole, thiophene, and triazole. Other heterocycles include morpholine, piprazine, piperidine, thiomorpholine, and thioazolidine. The heterocycles may be substituted or unsubstituted. Examples of substituents include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

It will further be noted that, depending upon, e.g., the methods for isolating and purifying the compounds of the present invention, there may exist a number of polymorphs of each individual compound. As used herein, the term “polymorph” refers to a solid crystalline phase of a compound represented by Formulae I, II or III, resulting from the possibility of at least two different arrangements of the molecules of the compound in the solid state. Crystalline forms of SNRI-NMDA antagonists, e.g., bicifadine hydrochloride is of particular importance because they may be formulated in various oral unit dosage forms as for example as tablets or capsules for the treatment of genitourinary disorders in patients. Variations in crystal structure of a pharmaceutical drug substance may affect the dissolution, manufacturability and stability of a pharmaceutical drug product, specifically in a solid oral dosage form formulation. Therefore it may be preferred to produce SNRI-NMDA antagonists in a pure form consisting of a single, thermodynamically stable crystal structure. It has been determined, for example, that the bicifadine hydrochloride crystal structure produced in accordance with commonly utilized synthesis (see, e.g., U.S. Pat. No. 4,231,935), is not the most thermodynamically stable polymorphic form. Furthermore, it has been demonstrated that bicifadine hydrochloride of this polymorphic form undergoes conversion to a different polymorphic form when subjected to conventional manufacturing processes, such as grinding and milling. Since the commonly produced polymorphic form is not the most thermodynamically stable form of bicifadine hydrochloride, it could also undergo polymorph conversion over time. Therefore, the commonly produced polymorphic form of bicifadine hydrochloride has not been the optimal crystalline form for formulation into pharmaceutical drug products.

Polymorphs of a given compound will be different in crystal structure but identical in liquid or vapor states. Moreover, solubility, melting point, density, hardness, crystal shape, optical and electrical properties, vapor pressure, stability, etc., may all vary with the polymorphic form. Remington's Pharmaceutical Sciences, 18th Edition, Mack Publishing Co. (1990), Chapter 75, pages 1439-1443. Such polymorphs are also meant to be included in the scope of this invention. Varying polymorphs may be created, for example, by applying kinetic energy, e.g., by grinding, milling, or stirring, preferably at low temperature or by applying heat and subsequently cooling in a controlled manner. The compounds of the present invention may exist as a single polymorphic form or as a mixture of multiple polymorphic forms.

Furthermore, the compounds of the present invention may be suitable for silicon switching as described, e.g., in Drug Discovery Today 8(12): 551-6 (2003) “Chemistry challenges in lead optimization: silicon isosteres in drug discovery”. Briefly, it has recently been discovered that certain carbon atoms in organic compounds, such as the compounds of the present invention, may be replaced by silicon atoms without noticeable loss in activity. Accordingly, in one embodiment, the present invention is directed to an SNRI-NMDA antagonist as described herein, e.g., defined by formulae I, II, or III or shown in any of the tables herein, wherein one or more of the carbons in the molecule has been replaced by a silicon. The skilled artisan can readily determine which compounds are eligible for silicon switching, which carbons of such compounds may be replaced, and how to effect the switch using no more than routine experimentation found, e.g., in Drug Discovery Today 8(12): 551-6 (2003) “Chemistry challenges in lead optimization: silicon isosteres in drug discovery”, cited above.

The present invention also includes the treatment of a genitourinary disorder in a subject in need of treatment with a dual acting SNRI-NMDA antagonist, wherein the dual acting SNRI-NMDA antagonist includes a first amount of an SNRI and a second amount of an NMDA antagonist. The first amount and the second amount can both be a therapeutically effective amount. Alternatively, the first amount and the second amount together form a therapeutically effective amount.

Suitable SNRIs include, but are not limited to, venlafaxine (e.g., EFFEXOR), duloxetine (e.g., CYMBALTA), amoxapine, maprotiline, milnacipran, and derivatives thereof. Suitable NMDA antagonists include, but are not limited to, ketamine, dextromethorphan, memantine, MRZ 2/579, amantadine, methadone, dextropropoxyphene, and ketobemidone.

In one aspect, the present invention provides a method of treating overactive bladder in a subject. The method generally includes administering to the subject a therapeutically effective amount of a dual acting SNRI-NMDA antagonist, such that the overactive bladder is treated. In some embodiments, the dual acting SNRI-NMDA antagonist includes a first amount of an SNRI; and a second amount of an NMDA antagonist. The first amount and the second amount may both be therapeutically effective amounts. Alternatively, the first amount and the second amount may together form a therapeutically effective amount. In other embodiments, the dual acting SNRI-NMDA antagonist includes one agent having both SNRI activity and NMDA antagonist activity.

In preferred embodiments, the dual acting SNRI-NMDA antagonist has one of the following structural formulas:

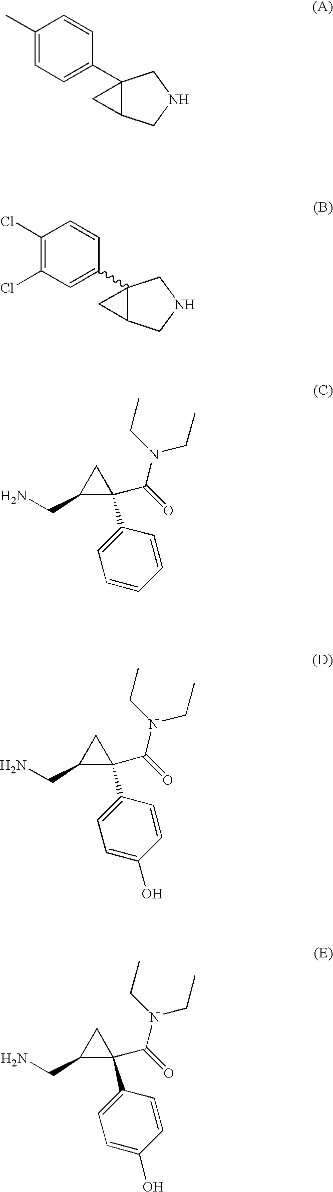

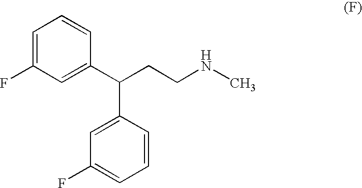

or pharmaceutically acceptable salts thereof.

In another embodiment, the method further comprises administering a therapeutically effective amount of an (i.e., one or more) additional therapeutic agent.

In another aspect, the present invention is directed to the use of a dual acting SNRI-NMDA antagonist for a genitourinary disorder to treat a disorder associated with control of the smooth muscle of the urinary bladder, e.g., urge urinary incontinence or nocturia.

Dual acting SNRI-NMDA antagonists, such as the compounds represented by structural Formulas I, II and III, are useful for treating at least one symptom associated with a genitourinary disorder by virtue of the triple therapeutic modes of action which they can exhibit. That is, the unique ability to modulate the function of the serotonin and noradrenaline reuptake mechanisms and the NMDA antagonist function can provide an enhanced treatment regimen for the subject undergoing treatment.

Combinatorial Libraries

The compounds of the present invention readily lend themselves to preparation using the methods of combinatorial chemistry, providing access to combinatorial libraries of compounds for the screening of pharmaceutical, agrochemical or other biological or medically-related activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO94/08051; Chen et al. (1994) JACS 116:2661: Kerr et al. (1993) JACS 115:252; PCT publications WO92/10092, WO93/09668 and WO91/07087; and the Lemer et al. PCT publication WO93/20242). Accordingly, a variety of libraries on the order of about 16 to 1,000,000 or more diversomers can be synthesized and screened for a particular activity or property.

In an exemplary embodiment, a library of substituted diversomers can be synthesized using reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. Detailed descriptions of a number of combinatorial methodologies are provided below.

Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

Multipin Synthesis

Libraries may also take a multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different-region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

Encoded Combinatorial Libraries

In yet another embodiment, a compound library provided with an encoded tagging system may be utilized. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-Oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115 :2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-Sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

Resolution of Enantiomers

Certain compounds of the present invention may exist in particular geometric or stereoisomeric forms. The present invention contemplates all such compounds, including cis- and trans-isomers, R— and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One alternative procedure for the isolation of an individual enantiomer is by resolution of an enantiomer from a racemic mixture. Chiral separations of cationic drugs by capillary electrophoresis may generally be carried out by adding negatively charged cyclodextrins (CDs) to the running buffer, while anionic or neutral drug separations require the use of dual-CD systems (mixtures of neutral and charged CDs). Chiral separation of some basic drugs (idazoxan, efaroxan, milnacipran) has been studied by mixtures of sulfated-β-CD (S-β-CD) and hydroxypropyl-γ-CD (HP-γ-CD). The influence of the following parameters (nature and concentration of neutral CD, concentration of S-β-CD) on many separation factors (electrophoretic mobility, selectivity, efficiency, asymmetry factor, resolution) demonstrated that dual-CD systems are useful for chiral separation of basic drugs in order to improve the symmetry of the second-migrating enantiomer. Indeed, the neutral CD reduces the extent of electromigration dispersion by mobility tuning. Finally, the 0.5 mg/mL S-β-CD/S mg/mL HP-γ-CD dual system has allowed the chiral separation of idazoxan, efaroxan and milnacipran enantiomers in less than 9 min. See generally Grard, S. et al. Electrophoresis 2000, 21, 3028-3034.

In a further embodiment, the pharmaceutical composition further comprises an (i.e., one or more) additional therapeutic agent.

The combination of agents used within the methods and pharmaceutical compositions described herein may have a therapeutic additive or synergistic effect on the condition(s) or disease(s) targeted for treatment. The combination of agents used within the methods or pharmaceutical compositions described herein also may reduce a detrimental effect associated with at least one of the agents when administered alone or without the other agent(s) of the particular pharmaceutical composition. For example, the toxicity of side effects of one agent may be attenuated by another agent of the composition, thus allowing a higher dosage, improving patient compliance, and improving therapeutic outcome. Physicians may achieve the clinical benefits of previously recognized drugs while using lower dosage levels, thus minimizing adverse side effects. The additive or synergistic effects, benefits, and advantages of the compositions apply to classes of therapeutic agents, either structural or functional classes, or to individual compounds themselves.

An additional therapeutic agent suitable for use in the methods and pharmaceutical compositions described herein, can be, but is not limited to, for example: an antimuscarinic (e.g., oxybutynin, DITROPAN®, tolterodine, flavoxate, propiverine, trospium); a muscosal surface protectant (e.g., ELMIRON®); an antihistamine (e.g., hydroxyzine hydrochloride or pamoate); an anticonvulsant (e.g., NEURONTIN® and KLONOPIN®); a muscle relaxant (e.g., VALIUM®; a bladder antispasmodic (e.g., URIMAX®); a tricyclic antidepressant (e.g., imipramine); a nitric oxide donor (e.g., nitroprusside), a β₃-adrenergic receptor agonist, a bradykinin receptor antagonist, a neurokinin receptor antagonist, a sodium channel modulator, such as TTX-R sodium channel modulator and/or activity dependent sodium channel modulator and a Cav2.2 subunit calcium channel modulator. Generally, the additional therapeutic agent will be one that is useful for treating the disorder of interest. Preferably, the additional therapeutic agent does not diminish the effects of the primary agent(s) and/or potentiates the effect of the primary agent(s).

Use of an additional therapeutic agent in combination with the primary agent(s) (i.e., a dual acting SNRI-NMDA antagonist) can result in less of any of the primary agent(s) and/or less of the additional agent being needed to achieve therapeutic efficacy. In some instances, use of less of an agent can be advantageous in that it provides a reduction in undesirable side effects.

By the term “antimuscarinic agent” as used herein is intended any muscarinic acetylcholine receptor antagonist. Unless otherwise indicated, the terms “anticholinergic agent,” “antinicotinic agent,” and “antimuscarinic agent” are intended to include anticholinergic, antinicotinic, and antimuscarinic agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

More specifically, oxybutynin, also known as 4-diethylaminio-2-butynyl phenylcyclohexyglycolate is a preferred antimuscarinic agent. DITROPAN® (oxybutynin chloride) is the d,1 racemic mixture of oxybutynin, which is known to exert antispasmodic effect on smooth muscle and inhibit the muscarinic action of acetylcholine on smooth muscle. Metabolites and isomers of oxybutynin have also been shown to have activity useful according to the present invention. Examples include, but are not limited to N-desethyl-oxybutynin and S-oxybutynin (see, e.g., U.S. Pat. Nos. 5,736,577 and 5,532,278).

Additional compounds that have been identified as antimuscarinic agents and are useful in the present invention include, but are not limited to:
a. Darifenacin (Daryon®) or acids, salts, enantiomers, analogs, esters, amides, prodrugs, active metabolites, and derivatives thereof;
b. Solifenacin or acids, salts, enantiomers, analogs, esters, amides, prodrugs, active metabolites, and derivatives thereof;
c. YM-905 (solifenacin succinate) or acids, salts, enantiomers, analogs, esters, amides, prodrugs, active metabolites, and derivatives thereof;
d. Solifenacin monohydrochloride or acids, salts, enantiomers, analogs, esters, amides, prodrugs, active metabolites, and derivatives thereof;
e. Tolterodine (Detrol®) or acids, salts, enantiomers, analogs, esters, amides, prodrugs, active metabolites, and derivatives thereof;
f. Propiverine (Detrunorm®) or acids, salts, enantiomers, analogs, esters, amides, prodrugs, active metabolites, and derivatives thereof;
g. Propantheline bromide (Pro-Banthine®) or acids, salts, enantiomers, analogs, esters, amides, prodrugs, active metabolites, and derivatives thereof;
h. Hyoscyamine sulfate (Levsin®, Cystospaz®) or acids, salts, enantiomers, analogs, esters, amides, prodrugs, active metabolites, and derivatives thereof;
i. Dicyclomine hydrochloride (Bentyl®) or acids, salts, enantiomers, analogs, esters, amides, prodrugs, active metabolites, and derivatives thereof;
j. Flavoxate hydrochloride (Urispas®) or acids, salts, enantiomers, analogs, esters, amides, prodrugs, active metabolites, and derivatives thereof;
k. d,1 (racemic) 4-diethylamino-2-butynyl phenylcyclohexylglycolate or acids, salts, enantiomers, analogs, esters, amides, prodrugs, active metabolites, and derivatives thereof;
l. (R)-N,N-diisopropyl-3-(2-hydroxy-5-methylphenyl)-3-phenylpropanamine L-hydrogen tartrate or acids, salts, enantiomers, analogs, esters, amides, prodrugs, active metabolites, and derivatives thereof;
m. (+)-(1S,3′R)-quinuclidin-3′-yl-1-phenyl-1,2,3,4-tetrahydro-isoquinoline-2-carboxylate monosuccinate or acids, salts, enantiomers, analogs, esters, amides, prodrugs, active metabolites, and derivatives thereof;
n. alpha(+)-4-(Dimethylamino)-3-methyl-1,2-diphenyl-2-butanol proprionate or acids, salts, enantiomers, analogs, esters, amides, prodrugs, active metabolites, and derivatives thereof;
o. 1-methyl-4-piperidyl diphenylpropoxyacetate or acids, salts, enantiomers, analogs, esters, amides, prodrugs, active metabolites, and derivatives thereof;
p. 3-hydroxyspiro[1H,5H-nortropane-8,1′-pyrrolidinium benzilate or acids, salts, enantiomers, analogs, esters, amides, prodrugs, active metabolites, and derivatives thereof;
q. 4 amino-piperidine containing compounds as disclosed in Diouf et al. (2002) Bioorg. Med. Chem. Lett. 12: 2535-9;
r. pirenzipine or acids, salts, enantiomers, analogs, esters, amides, prodrugs, active metabolites, and derivatives thereof;
s. methoctramine or acids, salts, enantiomers, analogs, esters, amides, prodrugs, active metabolites, and derivatives thereof;
t. 4-diphenylacetoxy-N-methyl piperidine methiodide;
u. tropicamide or acids, salts, enantiomers, analogs, esters, amides, prodrugs, active metabolites, and derivatives thereof;
v. (2R)-N-[1-(6-aminopyridin-2-ylmethyl)piperidin-4-yl]-2-[(1R)-3,3-difluorocyclopentyl]-2-hydroxy-2-phenylacetamide or acids, salts, enantiomers, analogs, esters, amides, prodrugs, active metabolites, and derivatives thereof;
w. PNU-200577 ((R)-N,N-diisopropyl-3-(2-hydroxy-5-hydroxymethylphenyl)-3-phenylpropanamine) or acids, salts, enantiomers, analogs, esters, amides, prodrugs, active metabolites, and derivatives thereof;
x. KRP-197 (4-(2-methylimidazolyl)-2,2-diphenylbutyramide) or acids, salts, enantiomers, analogs, esters, amides, prodrugs, active metabolites, and derivatives thereof;
y. Fesoterodine or acids, salts, enantiomers, analogs, esters, amides, prodrugs, active metabolites, and derivatives thereof; and
z. SPM 7605 (the active metabolite of Fesoterodine), or acids, salts, enantiomers, analogs, esters, amides, prodrugs, active metabolites, and derivatives thereof.

The identification of further compounds that have antimuscarinic activity and would therefore be useful in the present invention can be determined by performing muscarinic receptor binding specificity studies as described by Nilvebrant (2002) Pharmacol. Toxicol. 90: 260-7 or cystometry studies as described by Modiri et al. (2002) Urology 59: 963-8.

The term “β₃ adrenergic receptor agonist” is used in its conventional sense to refer to a compound that binds to and agonizes β₃ adrenergic receptors. Unless otherwise indicated, the term β₃ adrenergic agonist” is intended to include β₃ adrenergic agonist agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Compounds that have been identified as β₃ adrenergic agonist agents and are useful in the present invention include, but are not limited to:
a. TT-138 and phenylethanolamine compounds as disclosed in U.S. Pat. No. 6,069,176, PCT Publication No. WO 97/15549 and available from Mitsubishi Pharma Corp., or acids, salts, esters, amides, prodrugs, active metabolites, and other derivatives thereof;
b. FR-149174 and propanolamine derivatives as disclosed in U.S. Pat. Nos. 6,495,546 and 6,391,915 and available from Fujisawa Pharmaceutical Co., or acids, salts, esters, amides, prodrugs, active metabolites, and other derivatives thereof;
c. KUC-7483, available from Kissei Pharmaceutical Co., or acids, salts, esters, amides, prodrugs, active metabolites, and other derivatives thereof,
d. 4′-hydroxynorephedrine derivatives such as 2-2-chloro-4-(2-((1S,2R)-2-hydroxy-2-(4-hydroxyphenyl)-1-methylethylamino)ethyl)-phenoxy acetic acid as disclosed in Tanaka et al. (2003) J. Med. Chem. 46: 105-12 or acids, salts, esters, amides, prodrugs, active metabolites, and other derivatives thereof;
e. 2-amino-1-phenylethanol compounds, such as BRL35135 ((R*R*)-(.±.)-[4-[2-[2-(3-chlorophenyl)-2-ydroxyethylamino]propyl]phenoxy]acetic acid methyl ester hydrobromide salt as disclosed in Japanese Patent Publication No. 26744 of 1988 and European Patent Publication No. 23385), and SR58611A ((RS)-N-(7-ethoxycarbonylmethoxy-1,2,3,4-tetrahydronaphth-2-yl)-2-(3-chlorophenyl)-2-hydroxyethanamine hydrochloride as disclosed in Japanese Laid-open Patent Publication No. 66152 of 1989 and European Laid-open Patent Publication No. 255415) or acids, salts, esters, amides, prodrugs, active metabolites, and other derivatives thereof;
f. GS 332 (Sodium (2R)-[3-[3-[2-(3-Chlorophenyl)-2-hydroxyethylamino]cyclohexyl]phenoxy]acetate) as disclosed in Iizuka et al. (1998) J. Smooth Muscle Res. 34: 139-49 or acids, salts, esters, amides, prodrugs, active metabolites, and other derivatives thereof;
g. BRL-37,344 (4-[-[(2-hydroxy-(3-chlorophenyl)ethyl)-amino]propyl]phenoxyacetate) as disclosed in Tsujii et al. (1998) Physiol. Behav. 63: 723-8 and available from GlaxoSmithKline or acids, salts, esters, amides, prodrugs, active metabolites, and other derivatives thereof; h. BRL-26830A as disclosed in Takahashi et al. (1992) Jpn Circ. J. 56: 936-42 and available from GlaxoSmithKline or acids, salts, esters, amides, prodrugs, active metabolites, and other derivatives thereof;
i. CGP 12177 (4-[3-t-butylamino-2-hydroxypropoxy]benzimidazol-2-one) (a 1/2 adrenergic antagonist reported to act as an agonist for the 3 adrenergic receptor) as described in Tavernier et al. (1992) J. Pharmacol. Exp. Ther. 263: 1083-90 and available from Ciba-Geigy or acids, salts, esters, amides, prodrugs, active metabolites, and other derivatives thereof;
j. CL 316243 (R,R-5-[2-[[2-(3-chlorophenyl)-2-hydroxyethyl]amino]propyl]-1,3-benzodioxole-2,2-dicarboxylate) as disclosed in Berlan et al. (1994) J. Pharmacol. Exp. Ther. 268: 1444-51 or acids, salts, esters, amides, prodrugs, active metabolites, and other derivatives thereof;
k. Compounds having 3 adrenergic agonist activity as disclosed in U.S. patent application 20030018061 or acids, salts, esters, amides, prodrugs, active metabolites, and other derivatives thereof;
l. ICI 215,001 HCl ((S)-4-[2-Hydroxy-3-phenoxypropyl-aminoethoxy]phenoxyacetic acid hydrochloride) as disclosed in Howe (1993) Drugs Future 18: 529 and available from AstraZeneca/ICI Labs or acids, salts, enantiomers, analogs, esters, amides, prodrugs, active metabolites, and derivatives thereof;
m. ZD 7114 HCl (ICI D7114; (S)-4-[2-Hydroxy-3-phenoxypropyl-aminoethoxy]-N-(2-methoxyethyl)phenoxyacetamide HCl) as disclosed in Howe (1993) Drugs Future 18: 529 and available from AstraZeneca/ICI Labs or acids, salts, enantiomers, analogs, esters, amides, prodrugs, active metabolites, and derivatives thereof;
n. Pindolol (1-(1H-Indol-4-yloxy)-3-[(1-methylethyl)amino]-2-propanol) as disclosed in Blin et al (1994) Mol. Pharmacol. 44: 1094 or acids, salts, enantiomers, analogs, esters, amides, prodrugs, active metabolites, and derivatives thereof;
o. (S)-(−)-Pindolol ((S)-1-(1H-indol-4-yloxy)-3-[(1-methylethyl)amino]-2-propanol) as disclosed in Walter et al (1984) Naunyn-Schmied. Arch. Pharmacol. 327: 159 and Kalkman (1989) Eur. J. Pharmacol. 173: 121 or acids, salts, enantiomers, analogs, esters, amides, prodrugs, active metabolites, and derivatives thereof;
p. SR 59230A HCl (1-(2-Ethylphenoxy)-3-[[(1S)-1,2,3,4-tetrahydro-1-naphthalenyl]amino]-(2S)-2-propanol hydrochloride) as disclosed in Manara et al. (1995) Pharmacol. Comm. 6: 253 and Manara et al. (1996) Br. J. Pharmacol. 117: 435 and available from Sanofi-Midy or acids, salts, enantiomers, analogs, esters, amides, prodrugs, active metabolites, and derivatives thereof;
q. SR 58611 (N[2s)7-carb-ethoxymethoxy-1,2,3,4-tetra-hydronaphth]-(2r)-2-hydroxy-2(3-chlorophenyl) ethamine hydrochloride) as disclosed in Gauthier et al. (1999) J. Pharmacol. Exp. Ther. 290: 687-693 and available from Sanofi Research; and
r. YM 178 available from Yamanouchi Pharmaceutical Co. or acids, salts, esters, amides, prodrugs, active metabolites, and other derivatives thereof.

The identification of further compounds that have β₃ adrenergic agonist activity and would therefore be useful in the present invention can be determined by performing radioligand binding assays and/or contractility studies as described by Zilberfarb et al. (1997) J. Cell Sci. 110: 801-807; Takeda et al. (1999) J. Pharmacol. Exp. Ther. 288: 1367-1373; and Gauthier et al. (1999) J. Pharmacol. Exp. Ther. 290: 687-693.

Further, agents for use as additional therapeutic agents include sodium channel modulators, such as TTX-R sodium channel modulators and/or activity dependent sodium channel modulators. TTX-R sodium channel modulators for use in the present invention include but are not limited to compounds that modulate or interact with Nav1.8 and/or Nav1.9 channels.

Sodium channel modulators suitable for use as in the practice of the invention include, but are not limited to propionamides such as Ralfinamide (NW-1029) (as disclosed in U.S. Pat. No. 5,236,957 and U.S. Pat. No. 5,391,577), which is also known as (+)-2(S)-[4-(2-Fluorobenzyloxy)benzylamino]propionamide and safinamide (as disclosed in U.S. Pat. Nos. 5,236,957 and U.S. Pat. No. 5,391,577), which is also known as 2(S)-[4-(3-Fluorobenzyloxy) benzylamino] propionamide methanesulfonate

Further sodium channel modulators include for example, N-phenylalkyl substituted “α-amino carboxamide derivatives in addition to Ralfinamide and Salfinamide as disclosed in U.S. Pat. No. 5,236,957; Other N-phenylalkyl substituted “α-amino carboxamide derivatives in addition to Ralfinamide and Salfinamide as disclosed in U.S. Pat. No. 5,391,577; Substituted 2-benzylamino-2-phenyl-acetamide compounds as disclosed in U.S. Pat. No. 6,303,819; aryldiazines and aryltriazines such as: sipatrigine (BW-619C; as disclosed in U.S. Pat. No. 5,684,005), which is also known as 4-Amino-2-(4-methylpiperazin-1-yl)-5-(2,3,5-trichlorophenyl)pyrimidine; 2-(4-Methylpiperazin-1-yl)-5-(2,3,5-trichlorophenyl)pyrimidine-4-amine ; lamotrigine (as disclosed in U.S. Pat. No. 4,602,017), which is also known as 6-(2,3-Dichlorophenyl)-1,2,4-triazine-3,5-diamine; GW-273293 (as disclosed in U.S. Pat. No. 6,599,905), which is also known as 3-(2,3,5-Trichlorophenyl)pyrazine-2,6-diamine; 4030W92 (as disclosed in U.S. Pat. No. 6,124,308), which is also known as 5-(2,3-Dichlorophenyl)-6-(fluoromethyl)pyrimidine-2,4-diamine; Carbamazepine (as disclosed in U.S. Pat. No. 2,948,718), which is also known as 5H-Dibenz[d,f]azepine-5-carboxamide; Oxcarbazepine (as disclosed in U.S. Pat. No. 3,642,775), which is also known as 10-Oxo-10,11-dihydro-5H-dibenz[b,f]azepine-5-carboxamide; licarbazepine (as disclosed in DE 2011045), which is also known as (±)-10-Hydroxy-10,11-dihydro-5H-dibenz[b,f]azepine-5-carboxamide; BIA-2-093 (as disclosed in U.S. Pat. No. 5,753,646), which is also known as Acetic acid 5-carbamoyl-10,11-dihydro-5H-dibenzo[b,f]azepin-10(S)-yl ester; ADCI (as disclosed in U.S. Pat. No. 5,196,415), which is also known as (+)-5,10-Imino-10,11-dihydro-5H-dibenzo[a,d]cycloheptene-5-carboxamide; Phenytoin sodium (as disclosed in U.S. Pat. No. 2,409,754) and OROS®-Phenytoin (as disclosed in U.S. Pat. No. 4,260,769), which are also known as 5,5-Diphenylhydantoin sodium salt and 5,5-Diphenyl-2,4-imidazolidinedione salt; Fosphenytoin sodium (as disclosed in U.S. Pat. No. 4,260,769) and phosphenytoin sodium, which are also known as 3-(Hydroxymethyl)-5,5-diphenylhydantoin phosphate ester disodium salt and 5,5-Diphenyl-3-[(phosphonooxy)methyl]-2,4-imidazolidinedione disodium salt; Pilsicainide hydrochloride and analogs thereof (as disclosed in U.S. Pat. No. 4,564,624), which is also known as N-(2,6-Dimethylphenyl)-8-pyrrolizidineacetamide hydrochloride; N-(2,6-Dimethylphenyl)-1-azabicyclo[3.3.0]octane-5-acetamide hydrochloride; Tocainide (as disclosed in DE 2235745), which is also known as 2-Amino-N-(2,6-dimethylphenyl)propanamide hydrochloride; Flecainide (as disclosed in U.S. Pat. No. 3,900,481), which is also known as N-(2-Piperidylmethyl)-2,5-bis(2,2,2-trifluoroethoxy)benzamide monoacetate; mexiletine hydrochloride (as disclosed in U.S. Pat. No. 3,954,872), which is also known as 1-(2,6-Dimethylphenoxy)-2-propanamine hydrochloride; Ropivacaine hydrochloride (as disclosed in PCT Publication No. WO 85/00599), which is also known as (−)-(S)-N-(n-Propyl)piperidine-2-carboxylic acid 2,6-xylidide hydrochloride monohydrate; (−)-(S)—N-(2,6-Dimethylphenyl)-1-propylpiperidine-2-carboxamide hydrochloride monohydrate; (−)-(S)-1-Propyl-2′,6′-pipecoloxylidide hydrochloride monohydrate; Lidocaine (as disclosed in U.S. Pat. No. 2,441,498), which is also known as 2-(diethylamino)-N-(2,6-dimethylphenyl)acetamide; mepivacaine (as disclosed in U.S. Pat. No. 2,799,679), which is also known as N-(2,6-dimethylphenyl)-1-methyl-2-piperidinecarboxamide; bupivacaine (as disclosed in U.S. Pat. No. 2,955,111), which is also known as 1-butyl-N-(2,6-dimethylphenyl)-2-piperidinecarboxamide; Prilocaine (as disclosed in U.S. Pat. No. 3,160,662), also known as N-(2-methylphenyl)-2-(propylamino)propanamide ; etidocaine (as disclosed in U.S. Pat. No. 3,812,147), which is also known as N-(2,6-dimethylphenyl)-1-methyl-2-piperidinecarboxamide; tetracaine (as disclosed in U.S. Pat. No. 1,889,645), which is also known as 4-(butylamino)benzoic acid 2-(diethylamino)ethyl ester; dibucaine (as disclosed in U.S. Pat. No. 1,825,623), which is also known as 2-butoxy-N-[2-(diethylamino)-ethyl]-4-quinolinecarboxamide; Soretolide, which is also known as 2,6-Dimethyl-N-(5-methylisozaxol-3-yl)benzamide; RS-132943 (as disclosed in U.S. Pat. No. 6,110,937), which is also known as 3(S)-(4-Bromo-2,6-dimethylphenoxymethyl)-1-methylpiperidine hydrochloride.

The identification of other agents that have affinity for TTX-R sodium channels or proteins associated with TTX-R sodium channels and would be useful in the present invention can be determined by methods that measure functional TTX-R channel activity such as sodium flux as disclosed in Stallcup, W B (1979) J. Physiol. 286: 525-40 or electrophysiological approaches as disclosed in Weiser and Wilson (2002) Mol. Pharmacol. 62: 433-438. The identification of other agents that exhibit activity-dependent modulation of sodium channels and would be useful in the present invention can be determined by methods as disclosed in Li et al., (1999) Molecular Pharmacology 55:134-141.

Further, agents for use as additional therapeutic agents include “Cav2.2 subunit calcium channel modulators” which are capable of binding to the Cav2.2 subunit of a calcium channel to produce a physiological effect, such as opening, closing, blocking, up-regulating expression, or down-regulating expression of the channel. Unless otherwise indicated, the term “Cav2.2 subunit calcium channel modulator” is intended to include amino acid compounds, peptide, nonpeptide, peptidomimetic, small molecular weight organic compounds, and other compounds that modulate or interact with the Cav2.2 subunit of a calcium channel (e.g., a binding event) or proteins associated with the Cav2.2 subunit of a calcium channel (e.g., a binding event) such as anchor proteins, as well as salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Cav2.2 subunit calcium channel modulator useful as an additional therapeutic agent in the practice of the invention include, but are not limited to:
a. ω-conotoxin GVIA or a salt, enantiomer, analog, ester, amide, prodrug, active metabolite, or derivative thereof;
b. ω-conotoxin MVIIA or a salt, enantiomer, analog, ester, amide, prodrug, active metabolite, or derivative thereof;
c. ω-conotoxin CNVIIA or a salt, enantiomer, analog, ester, amide, prodrug, active metabolite, or derivative thereof;
d. ω-conotoxin CVIID or a salt, enantiomer, analog, ester, amide, prodrug, active metabolite, or derivative thereof;
e. ω-conotoxin AM336 or a salt, enantiomer, analog, ester, amide, prodrug, active metabolite, or derivative thereof;
f. Cilnidipine or a salt, enantiomer, analog, ester, amide, prodrug, active metabolite, or derivative thereof;
g. Amlodipine or a salt, enantiomer, analog, ester, amide, prodrug, active metabolite, or derivative thereof;
h. L-cysteine derivative 2A or a salt, enantiomer, analog, ester, amide, prodrug, active metabolite, or derivative thereof;
i. ω-agatoxin IVA or a salt, enantiomer, analog, ester, amide, prodrug, active metabolite, or derivative thereof;
j. N,N-dialkyl-dipeptidylamines or a salt, enantiomer, analog, ester, amide, prodrug, active metabolite, or derivative thereof;
k. Levetiracetam or a salt, enantiomer, analog, ester, amide, prodrug, active metabolite, or derivative thereof; and
l. Ziconotide (SNX-111) or a salt, enantiomer, analog, ester, amide, prodrug, active metabolite, or derivative thereof;
m. (S)-alpha-ethyl-2-oxo-1-pyrrolidineacetamide (illustrated below) and disclosed in U.S. Pat. Nos. 4,943,639, 4,837,223, and 4,696,943, or a salt, enantiomer, analog, ester, amide, prodrug, active metabolite, or derivative thereof;
n. Substituted peptidylamines as disclosed in PCT Publication No. WO 98/54123, or a salt, enantiomer, analog, ester, amide, prodrug, active metabolite, or derivative thereof;
o. PD-173212 or a salt, enantiomer, analog, ester, amide, prodrug, active metabolite, or derivative thereof;
p. Reduced dipeptide analogues as disclosed in U.S. Pat. No. 6,316,440 and PCT Publication No. WO 00/06559, or a salt, enantiomer, analog, ester, amide, prodrug, active metabolite, or derivative thereof;
q. Amino acid derivatives as disclosed in PCT Publication No. WO 99/02146, or a salt, enantiomer, analog, ester, amide, prodrug, active metabolite, or derivative thereof;
r. Benzazepine derivatives as disclosed in Japanese Publication No. JP 2002363163, or a salt, enantiomer, analog, ester, amide, prodrug, active metabolite, or derivative thereof;
s. Compounds disclosed in PCT Publication No. WO 02/36567, or a salt, enantiomer, analog, ester, amide, prodrug, active metabolite, or derivative thereof;
t. Compounds disclosed in PCT Publication No. WO 03/018561, or a salt, enantiomer, analog, ester, amide, prodrug, active metabolite, or derivative thereof;
u. Compounds disclosed in U.S. patent Publication No. 2004009991 and PCT Publication No. WO 02/22588, or a salt, enantiomer, analog, ester, amide, prodrug, active metabolite, or derivative thereof;
v. Dihydropyridine derivatives as disclosed in U.S. Pat. No. 6,610,717, U.S. patent Publication No. 2002193605, and PCT Publication No. WO 00/78720, or a salt, enantiomer, analog, ester, amide, prodrug, active metabolite, or derivative thereof; and
w. Diarylalkene and diarylalkane derivatives as disclosed in PCT Publication No. WO 03/018538, or a salt, enantiomer, analog, ester, amide, prodrug, active metabolite, or derivative thereof.

Additional Cav2.2 subunit calcium channel modulator useful as an additional therapeutic agent in the practice of the invention include, but are not limited to non-peptide, and peptidomimetic drug-like molecules that bind to Cav2.2-containing calcium channels as disclosed in Lewis et al. (2000) J. Biol. Chem. 10: 35335-44; Smith et al. (2002) Pain 96: 119-27; Takahara et al. (2002) Eur. J. Pharmacol. 434: 43-7; Favreau et al. (2001) Biochemistry, 40: 14567-575; Seko et al. (2001) Bioorg. Med. Chem. Lett. 11: 2067-70; Hu et al. (2000) Bioorg. Med. Chem. Lett. 8: 1203-12; Lew et al. (1997) J. Biol. Chem. 272: 12014-23. It is understood that the present invention also encompasses any pharmaceutically acceptable, pharmacologically active salts, enantiomers, analogs, esters, amides, prodrugs, active metabolites, and derivatives of the aforementioned compounds.

The identification of other agents that have affinity for the Cav2.2 subunit of a calcium channel and would be useful in the present invention can be determined by performing Cav2.2 subunit binding affinity, electrophysiolgic, and/or other screening methods as described in Feng et al. (J. Biol. Chem., 278: 20171-20178, 2003), Feng et al. (J. Biol. Chem., 276: 15728-15735, 2001), Favreau et al. (Biochemistry, 40: 14567-575, 2001), and/or U.S. Pat. No. 6,387,897 assigned to NeuroMed Technologies Inc.

The term “spasmolytic” (also known as “antispasmodic”) is used in its conventional sense to refer to a compound that relieves or prevents muscle spasms, especially of smooth muscle. Unless otherwise indicated, the term “spasmolytic” is intended to include spasmolytic agents as disclosed further herein, as well as acids, salts, esters, amides, prodrugs, active metabolites, and other derivatives thereof.

Further, it is understood that any salts, esters, amides, prodrugs, active metabolites or other derivatives are pharmaceutically acceptable as well as pharmacologically active. In general, spasmolytics have been implicated as having efficacy in the treatment of bladder disorders (See. e.g., Takeda et al. (2000) J. Pharmacol. Exp. Ther. 293: 939-45).

Compounds that have been identified as spasmolytic agents and are useful in the present invention include, but are not limited to:
a. α-α-diphenylacetic acid-4-(N-methyl-piperidyl) esters as disclosed in U.S. Pat. No. 5,897,875 or acids, salts, enantiomers, analogs, esters, amides, prodrugs, active metabolites, and derivatives thereof;
b. Human and porcine spasmolytic polypeptides in glycosylated form and variants thereof as disclosed in U.S. Pat. No. 5,783,416 or acids, salts, enantiomers, analogs, esters, amides, prodrugs, active metabolites, and derivatives thereof;
c. Dioxazocine derivatives as disclosed in U.S. Pat. No. 4,965,259 or acids, salts, enantiomers, analogs, esters, amides, prodrugs, active metabolites, and derivatives thereof;
d. Quaternary 6,11-dihydro-dibenzo-[b,e]-thiepine-11-N-alkylnorscopine ethers as disclosed in U.S. Pat. No. 4,608,377 or acids, salts, enantiomers, analogs, esters, amides, prodrugs, active metabolites, and derivatives thereof;
e. Quaternary salts of dibenzo[1,4]diazepinones, pyrido-[1,4]benzodiazepinones, pyrido[1,5]benzodiazepinones as disclosed in U.S. Pat. No. 4,594,190 or acids, salts, enantiomers, analogs, esters, amides, prodrugs, active metabolites, and derivatives thereof;
f. Endo-8,8-dialkyl-8-azoniabicyclo (3.2.1) octane-6,7-exo-epoxy-3-alkyl-carboxylate salts as disclosed in U.S. Pat. No. 4,558,054 or acids, salts, enantiomers, analogs, esters, amides, prodrugs, active metabolites, and derivatives thereof;
g. Pancreatic spasmolytic polypeptides as disclosed in U.S. Pat. No. 4,370,317 or acids, salts, enantiomers, analogs, esters, amides, prodrugs, active metabolites, and derivatives thereof;
h. Triazinones as disclosed in U.S. Pat. No. 4,203,983 or acids, salts, enantiomers, analogs, esters, amides, prodrugs, active metabolites, and derivatives thereof;
i. 2-(4-Biphenylyl)-N-(2-diethylamino alkyl)propionamide as disclosed in U.S. Pat. No. 4,185,124 or acids, salts, enantiomers, analogs, esters, amides, prodrugs, active metabolites, and derivatives thereof;
j. Piperazino-pyrimidines as disclosed in U.S. Pat. No. 4,166,852 or acids, salts, enantiomers, analogs, esters, amides, prodrugs, active metabolites, and derivatives thereof;
k. Aralkylamino carboxylic acids as disclosed in U.S. Pat. No. 4,163,060 or acids, salts, enantiomers, analogs, esters, amides, prodrugs, active metabolites, and derivatives thereof;
l. Aralkylamino sulfones as disclosed in U.S. Pat. No. 4,034,103 or acids, salts, enantiomers, analogs, esters, amides, prodrugs, active metabolites, and derivatives thereof;
m. Smooth muscle spasmolytic agents as disclosed in U.S. Pat. No. 6,207,852 or acids, salts, enantiomers, analogs, esters, amides, prodrugs, active metabolites, and derivatives thereof; and
n. Papaverine or acids, salts, enantiomers, analogs, esters, amides, prodrugs, active metabolites, and derivatives thereof.

The identification of fuirther compounds that have spasmolytic activity and would therefore be useful in the present invention can be determined by performing bladder strip contractility studies as described in U.S. Pat. No. 6,207,852; Noronha-Blob et al. (1991) J. Pharmacol. Exp. Ther.256: 562-567; and/or Kachur et al. (1988) J. Pharmacol. Exp. Ther. 247: 867-872.

The term “tachykinin receptor antagonist” is used in its conventional sense to refer to a compound that binds to and antagonizes tachykinin receptors. Unless otherwise indicated, the term “tachykinin receptor antagonist” is intended to include tachykinin receptor antagonist agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Suitable tachykinin receptor antagonists for use in the present invention that act on the NK1 receptor include, but are not limited to: 1-imino-2-(2-methoxy-phenyl)-ethyl)-7,7-diphenyl-4-perhydroisoindolone(3aR ,7aR) (“RP 67580”); 2S,3S-cis-3-(2-methoxybenzylamino)-2-benzhydrylquinuclidine (“CP 96,345”); (aR,9R)-7-[3,5-bis(trifluoromethyl)benzyl]-8,9,10,11-tetrahydro-9-methyl-5-(4-methylphenyl)-7H-[1,4]diazocino[2,1-g] [1,7]naphthyridine-6,13-dione)(“TAK-637”); R-673; GW597599; Emend (MK-0869); CJ-11,974 and NKP-608. Suitable tachykinin receptor antagonists for use in the present invention that act on the NK2 receptor include but are not limited to: ((S)-N-methyl-N-4-(4-acetylamino-4-phenylpiperidino)-2-(3,4-dichlorophenyl)butylbenzamide (“SR 48968”); Met-Asp-Trp-Phe-Dap-Leu (“MEN 10,627”); and cyc(Gln-Trp-Phe-Gly-Leu-Met) (“L-659,877”). Suitable tachykinin receptor antagonists for use in the present invention that act on the NK3 receptor include but are not limited to talnetant and osanetant (SR 142801). Suitable tachykinin receptor antagonists for use in the present invention also include acids, salts, esters, amides, prodrugs, active metabolites, and other derivatives of any of the agents mentioned above. The identification of fuirther compounds that have tachykinin receptor antagonist activity and would therefore be useful in the present invention can be determined by performing binding assay studies as described in Hopkins et al. (1991) Biochem. Biophys. Res. Comm. 180: 1110-1117; and Aharony et al. (1994) Mol. Pharmacol. 45: 9-19.

The term “bradykinin receptor antagonist” is used in its conventional sense to refer to a compound that binds to and antagonizes bradykinin receptors. Unless otherwise indicated, the term “bradykinin receptor antagonist” is intended to include bradykinin receptor antagonist agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Suitable bradykinin receptor antagonists for use in the present invention that act on the BI receptor include but are not limited to: des-arg10HOE 140 (available from Hoechst Pharmaceuticals) and des-Arg9bradykinin (DABK). Suitable bradykinin receptor antagonists for use in the present invention that act on the B2 receptor include but are not limited to: D-Phe7-BK; D-Arg-(Hyp3-Thi5,8-D-Phe7)-BK (“NPC 349”); D-Arg-(Hyp3-D-Phe7)-BK (“NPC 567”); D-Arg-(Hyp3-ThiS -D-Tic7-Oic8)-BK (“HOE 140”); H-DArg-Arg-Pro-Hyp-Gly-Thi-c(Dab-DTic-Oic-Arg)c(7gamma-10alpha)(“MEN 11270”); H-DArg-Arg-Pro-Hyp-Gly-Thi-Ser-DTic-Oic-Arg-OH(“Icatibant”); (E)-3-(6-acetamido-3-pyridyl)-N-[N-[2,4-dichloro-3-[(2-methyl-8-quinolinyl)oxymethyl] phenyl]-N-methylaminocarbonylmethyl]acrylamide (“FRI73567”); and WIN 64338. These compounds are more fully described in Perkins, M. N., et. al., Pain, supra; Dray, A., et. al., Trends Neurosci., supra; and Meini et al. (2000) Eur. J. Pharmacol. 388: 177-82. Suitable neurokinin receptor antagonists for use in the present invention also include acids, salts, esters, amides, prodrugs, active metabolites, and other derivatives of any of the agents mentioned above. The identification of further compounds that have bradykinin receptor antagonist activity and would therefore be useful in the present invention can be determined by performing binding assay studies as described in Manning et al. (1986) J. Pharmacol. Exp. Ther. 237: 504 and U.S. Pat. No. 5,686,565.

The term “nitric oxide donor” is used in its conventional sense to refer to a compound that releases free nitric oxide when administered to a patient. Unless otherwise indicated, the term “nitric oxide donor” is intended to include nitric oxide donor agents as disclosed further herein, as well as acids, salts, esters, amides, prodrugs, active metabolites, and other derivatives thereof. Further, it is understood that any salts, esters, amides, prodrugs, active metabolites or other derivatives are pharmaceutically acceptable as well as pharmacologically active.

Suitable nitric oxide donors for the practice of the present invention include but are not limited to:
a. Nitroglycerin or acids, salts, enantiomers, analogs, esters, amides, prodrugs, active metabolites, and derivatives thereof;
b. Sodium nitroprusside or acids, salts, enantiomers, analogs, esters, amides, prodrugs, active metabolites, and derivatives thereof;
c. FK 409 (NOR-3) or acids, salts, enantiomers, analogs, esters, amides, prodrugs, active metabolites, and derivatives thereof;
d. FR 144420 (NOR-4) or acids, salts, enantiomers, analogs, esters, amides, prodrugs, active metabolites, and derivatives thereof;
e. 3-morpholinosydnonimine or acids, salts, enantiomers, analogs, esters, amides, prodrugs, active metabolites, and derivatives thereof;
f. Linsidomine chlorohydrate (“SIN-1”) or acids, salts, enantiomers, analogs, esters, amides, prodrugs, active metabolites, and derivatives thereof;
g. S-nitroso-N-acetylpenicillamine (“SNAP”) or acids, salts, enantiomers, analogs, esters, amides, prodrugs, active metabolites, and derivatives thereof;
h. AZD3582 (CINOD lead compound, available from NicOx S.A.) or acids, salts, enantiomers, analogs, esters, amides, prodrugs, active metabolites, and derivatives thereof;
i. NCX 4016 (available from NicOx S.A.) or acids, salts, enantiomers, analogs, esters, amides, prodrugs, active metabolites, and derivatives thereof;
j. NCX 701 (available from NicOx S.A.) or acids, salts, enantiomers, analogs, esters, amides, prodrugs, active metabolites, and derivatives thereof;
k. NCX 1022 (available from NicOx S.A.) or acids, salts, enantiomers, analogs, esters, amides, prodrugs, active metabolites, and derivatives thereof;
l. HCT 1026 (available from NicOx S.A.) or acids, salts, enantiomers, analogs, esters, amides, prodrugs, active metabolites, and derivatives thereof;
m. NCX 1015 (available from NicOx S.A.) or acids, salts, enantiomers, analogs, esters, amides, prodrugs, active metabolites, and derivatives thereof;
n. NCX 950 (available from NicOx S.A.) or acids, salts, enantiomers, analogs, esters, amides, prodrugs, active metabolites, and derivatives thereof;
o. NCX 1000 (available from NicOx S.A.) or acids, salts, enantiomers, analogs, esters, amides, prodrugs, active metabolites, and derivatives thereof;
p. NCX 1020 (available from NicOx S.A.) or acids, salts, enantiomers, analogs, esters, amides, prodrugs, active metabolites, and derivatives thereof;
q. AZD 4717 (available from NicOx S.A.) or acids, salts, enantiomers, analogs, esters, amides, prodrugs, active metabolites, and derivatives thereof;
r. NCX 151 O/NCX 1512 (available from NicOx S.A.) or acids, salts, enantiomers, analogs, esters, amides, prodrugs, active metabolites, and derivatives thereof;
s. NCX 2216 (available from NicOx S.A.) or acids, salts, enantiomers, analogs, esters, amides, prodrugs, active metabolites, and derivatives thereof;
t. NCX 4040 (available from NicOx S.A.) or acids, salts, enantiomers, analogs, esters, amides, prodrugs, active metabolites, and derivatives thereof;
u. Nitric oxide donors as disclosed in U.S. Pat. No. 5,155,137 or acids, salts, enantiomers, analogs, esters, amides, prodrugs, active metabolites, and derivatives thereof;
v. Nitric oxide donors as disclosed in U.S. Pat. No. 5,366,997 or acids, salts, enantiomers, analogs, esters, amides, prodrugs, active metabolites, and derivatives thereof;
w. Nitric oxide donors as disclosed in U.S. Pat. No. 5,405,919 or acids, salts, enantiomers, analogs, esters, amides, prodrugs, active metabolites, and derivatives thereof;
x. Nitric oxide donors as disclosed in U.S. Pat. No. 5,650,442 or acids, salts, enantiomers, analogs, esters, amides, prodrugs, active metabolites, and derivatives thereof;
y. Nitric oxide donors as disclosed in U.S. Pat. No. 5,700,830 or acids, salts, enantiomers, analogs, esters, amides, prodrugs, active metabolites, and derivatives thereof;
z. Nitric oxide donors as disclosed in U.S. Pat. No. 5,632,981 or acids, salts, enantiomers, analogs, esters, amides, prodrugs, active metabolites, and derivatives thereof;
aa. Nitric oxide donors as disclosed in U.S. Pat. No. 6,290,981 or acids, salts, enantiomers, analogs, esters, amides, prodrugs, active metabolites, and derivatives thereof;
bb. Nitric oxide donors as disclosed in U.S. Pat. No. 5,691,423 or acids, salts, enantiomers, analogs, esters, amides, prodrugs, active metabolites, and derivatives thereof;
cc. Nitric oxide donors as disclosed in U.S. Pat. No. 5,721,365 or acids, salts, enantiomers, analogs, esters, amides, prodrugs, active metabolites, and derivatives thereof;
dd. Nitric oxide donors as disclosed in U.S. Pat. No. 5,714,511 or acids, salts, enantiomers, analogs, esters, amides, prodrugs, active metabolites, and derivatives thereof;
ee. Nitric oxide donors as disclosed in U.S. Pat. No. 6,511,911 or acids, salts, enantiomers, analogs, esters, amides, prodrugs, active metabolites, and derivatives thereof; and
ff. Nitric oxide donors as disclosed in U.S. Pat. No. 5,814,666.

The identification of further compounds that have nitric oxide donor activity and would therefore be useful in the present invention can be determined by release profile and/or induced vasospasm studies as described in U.S. Pat. Nos. 6,451,337 and 6,358,536, as well as Moon (2002) IBJU Int. 89: 942-9 and Fathian-Sabet et al. (2001) J. Urol. 165: 1724-9.

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In preferred embodiments, the subject does not have a chemical dependency.

“Treatment”, or “treating” as used herein, is defined as the application or administration of a therapeutic agent (e.g., a dual acting SNRI-NMDA antagonist of the invention) to a subject, who has a genitourinary disorder, e.g., overactive bladder, a symptom of a genitourinary disorder, e.g., overactive bladder, or a predisposition toward a genitourinary disorder, e.g., overactive bladder, with the purpose to cure, heal, alleviate, relieve, alter, remedy, ameliorate, improve or affect the genitourinary disorder, the symptoms of the genitourinary disorder, or the predisposition toward the genitourinary disorder. “Treated,” as used herein, refers to the genitourinary disorder or at least one symptom of the genitourinary disorder being cured, healed, alleviated, relieved, altered, remedied, ameliorated improved or affected. For example, certain methods of treatment of the instant invention provide for administration of a dual acting SNRI-NMDA antagonist, such that urgency due to a genitourinary disorder is improved or alleviated.

The terms “cure,” “heal,” “alleviate,” “relieve,” “alter,” “remedy,” “ameliorate,” “improve” and “affect” are evaluated in terms of a suitable or appropriate control. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to administration of a dual acting SNRI-NMDA antagonist, as described herein. For example, the level of urgency can be determined prior to administration of a dual acting SNRI-NMDA antagonist of the invention to a subject. In another embodiment, a “suitable control” or “appropriate control” is a value, level, feature, characteristic, property, etc. determined in another subject, e.g., a control or normal subject exhibiting, for example, normal traits. In yet another embodiment, a “suitable control” or “appropriate control” is a predefined value, level, feature, characteristic, property, etc.

As used herein, therapeutically effective amount refers to an amount sufficient to elicit the desired biological response. In the present invention the desired biological response is a reduction (complete or partial) of at least one symptom associated with the genitourinary disorder being treated. As with any treatment, particularly treatment of a multi-symptom disorder, for example, overactive bladder, it is advantageous to treat as many disorder-related symptoms which the subject experiences.

Pharmaceutically acceptable carrier includes pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Pharmaceutically acceptable carriers can be aqueous or non-aqueous solvents. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media.

Modes of Administration

The compounds for use in the method or kits of the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intraduodenal, intrathecal, subcutaneous, intramuscular, intradermal, intra-arterial, intravenous, inhalation, and topical administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. Further, those of ordinary skill in the art can readily deduce that suitable formulations involving these compositions and dosage forms, including those formulations as described elsewhere herein.

The term intravesical administration is used herein in its conventional sense to mean delivery of a drug directly into the bladder.

For oral administration the compounds can be of the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY® film coating systems available from Colorcon, West Point, Pa. (e.g., OPADRY® OY Type, OY-C Type, Organic Enteric OY-P Type, Aqueous Enteric OY-A Type, OY-PM Type and OPADRY® White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

The dosage form may also be a capsule, in which case the active agent-containing composition may be encapsulated in the form of a liquid or solid (including particulates such as granules, beads, powders or pellets). Suitable capsules can be hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals. If the active agent-containing composition is present within the capsule in liquid form, a liquid carrier can be used to dissolve the active agent(s). The carrier should be compatible with the capsule material and all components of the pharmaceutical composition, and should be suitable for ingestion.

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

Preferred buccal dosage forms will typically comprise a therapeutically effective amount of an active agent and a bioerodible (hydrolyzable) polymeric carrier that may also serve to adhere the dosage form to the buccal mucosa. The buccal dosage unit can be fabricated so as to erode over a predetermined time period, wherein drug delivery is provided essentially throughout. The time period is typically in the range of from about 1 hour to about 72 hours. Preferred buccal delivery occurs over a time period of from about 2 hours to about 24 hours. Buccal drug delivery for short term use should preferably occur over a time period of from about 2 hours to about 8 hours, more preferably over a time period of from about 3 hours to about 4 hours. As needed, buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The amount of the active agent in the buccal dosage unit will of course depend on the potency of the agent and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt. % active agent.

With regard to the bioerodible (hydrolyzable) polymeric carrier, it will be appreciated that virtually any such carrier can be used, so long as the desired drug release profile is not compromised, and the carrier is compatible with the active agents to be administered and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components can also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmelose (e.g., Ac-di-sol®, which may be obtained from FMC), alginic acid, and sodium carboxymethyl starches (e.g., Explotab®, which can be obtained from Edward Medell Co., Inc.), methylcellulose, agar bentonite and alginic acid. Suitable diluents include those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, include those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include tablets, creams, ointments, lozenges, pastes, and any other suitable dosage form where the active ingredient is admixed into a disintegrable matrix. The tablet, cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units can be fabricated to disintegrate rapidly. The time period for complete disintegration of the dosage unit is typically in the range of from about 10 seconds to about 30 minutes, and optimally is less than 5 minutes.

Other components can also be incorporated into the sublingual and lingual dosage forms described herein. The additional components include, but are not limited to binders, disintegrants, wetting agents, lubricants, and the like. Examples of binders that can be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like.

Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy, supra).

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials, with polyethylene glycol and derivatives thereof particularly preferred. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone® from Nelson Research & Development Co., Irvine, Calif.), SEPA® (available from Macrochem Co., Lexington, Mass.), surfactants as discussed above, including, for example, Tergitol®, Nonoxynol-9® and TWEEN-80®, and lower alkanols such as ethanol.

Transurethral drug administration, as explained in U.S. Pat. Nos. 5,242,391, 5,474,535, 5,686,093 and 5,773,020, can be carried out in a number of different ways using a variety of urethral dosage forms. For example, the drug can be introduced into the urethra from a flexible tube, squeeze bottle, pump or aerosol spray. The drug can also be contained in coatings, pellets or suppositories that are absorbed, melted or bioeroded in the urethra. In certain embodiments, the drug is included in a coating on the exterior surface of a penile insert. It is preferred, although not essential, that the drug be delivered from at least about 3 cm into the urethra, and preferably from at least about 7 cm into the urethra. Generally, delivery from at least about 3 cm to about 8 cm into the urethra will provide effective results in conjunction with the present method.

Urethral suppository formulations containing PEG or a PEG derivative can be conveniently formulated using conventional techniques, e.g., compression molding, heat molding or the like, as will be appreciated by those skilled in the art and as described in the pertinent literature and pharmaceutical texts. (See, e.g., Remington: The Science and Practice of Pharmacy, supra), which discloses typical methods of preparing pharmaceutical compositions in the form of urethral suppositories. The PEG or PEG derivative preferably has a molecular weight in the range of from about 200 to about 2,500 g/mol, more preferably in the range of from about 1,000 to about 2,000 g/mol.

Suitable polyethylene glycol derivatives include polyethylene glycol fatty acid esters, for example, polyethylene glycol monostearate, polyethylene glycol sorbitan esters, e.g., polysorbates, and the like. Depending on the particular active agent, urethral suppositories may contain one or more solubilizing agents effective to increase the solubility of the active agent in the PEG or other transurethral vehicle.

It may be desirable to deliver the active agent in a urethral dosage form that provides for controlled or sustained release of the agent. In such a case, the dosage form can comprise a biocompatible, biodegradable material, typically a biodegradable polymer. Examples of such polymers include polyesters, polyalkylcyanoacrylates, polyorthoesters, polyanhydrides, albumin, gelatin and starch. As explained, for example, in PCT Publication No. WO 96/40054, these and other polymers can be used to provide biodegradable microparticles that enable controlled and sustained drug release, in turn minimizing the required dosing frequency.

The urethral dosage form will preferably comprise a suppository that is from about 2 to about 20 mm in length, preferably from about 5 to about 10 mm in length, and less than about 5 mm in width, preferably less than about 2 mm in width. The weight of the suppository will typically be in the range of from about 1 mg to about 100 mg, preferably in the range of from about 1 mg to about 50 mg. However, it will be appreciated by those skilled in the art that the size of the suppository can and will vary, depending on the potency of the drug, the nature of the formulation, and other factors.

Transurethral drug delivery may involve an “active” delivery mechanism such as iontophoresis, electroporation or phonophoresis. Devices and methods for delivering drugs in this way are well known in the art. Iontophoretically assisted drug delivery is, for example, described in PCT Publication No. WO 96/40054, cited above. Briefly, the active agent is driven through the urethral wall by means of an electric current passed from an external electrode to a second electrode contained within or affixed to a urethral probe.

Preferred transrectal dosage forms can include rectal suppositories, creams, ointments, and liquid formulations (enemas). The suppository, cream, ointment or liquid formulation for transrectal delivery comprises a therapeutically effective amount of the selected phosphodiesterase inhibitor and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours.

The time period for complete disintegration is preferably in the range of from about 10 minutes to about 6 hours, and optimally is less than about 3 hours.

Other components can also be incorporated into the transrectal dosage forms described herein. The additional components include, but are not limited to, stiffening agents, antioxidants, preservatives, and the like. Examples of stiffening agents that may be used include, for example, paraffin, white wax and yellow wax. Preferred antioxidants, if used, include sodium bisulfite and sodium metabisulfite.

Preferred vaginal or perivaginal dosage forms include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration is preferably in the range of from about 10 minutes to about 6 hours, and optimally is less than about 3 hours.

Other components can also be incorporated into the vaginal or perivaginal dosage forms described herein. The additional components include, but are not limited to, stiffening agents, antioxidants, preservatives, and the like. Examples of stiffening agents that may be used include, for example, paraffin, white wax and yellow wax.

Preferred antioxidants, if used, include sodium bisulfite and sodium metabisulfite.

The active agents can also be administered intranasally or by inhalation.

Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. Preferably, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations can be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent.

Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof).

Non-aerosol formulations for inhalation can also comprise dry powder formulations, particularly insufflations in which the powder has an average particle size of from about 0.1 μm to about 50 μm, preferably from about 1 μm to about 25 μm.

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

Another system available from Medtronic that is commonly utilized for intrathecal administration is the fully implantable, programmable SynchroMed® Infusion System. The SynchroMed® Infusion System has two parts that are both placed in the body during a surgical procedure: the catheter and the pump. The catheter is a small, soft tube. One end is connected to the catheter port of the pump, and the other end is placed in the intrathecal space. The pump is a round metal device about one inch (2.5 cm) thick, three inches (8.5 cm) in diameter, and weighs about six ounces (205 g) that stores and releases prescribed amounts of medication directly into the intrathecal space. It can be made of titanium, a lightweight, medical-grade metal. The reservoir is the space inside the pump that holds the medication. The fill port is a raised center portion of the pump through which the pump is refilled. The doctor or a nurse inserts a needle through the patient's skin and through the fill port to fill the pump. Some pumps have a side catheter access port that allows the doctor to inject other medications or sterile solutions directly into the catheter, bypassing the pump.

The SynchroMed® pump automatically delivers a controlled amount of medication through the catheter to the intrathecal space around the spinal cord, where it is most effective. The exact dosage, rate and timing prescribed by the doctor are entered in the pump using a programmer, an external computer-like device that controls the pump's memory. Information about the patient's prescription can be stored in the pump's memory. The doctor can easily review this information by using the programmer. The programmer communicates with the pump by radio signals that allow the doctor to tell how the pump is operating at any given time. The doctor also can use the programmer to change your medication dosage.

Methods of intrathecal administration can include those described above available from Medtronic, as well as other methods that are known to one of skill in the art.

Suitable methods for intravesical administration can be found in U.S. Pat. Nos. 6,207,180 and 6,039,967, for example.

For other parenteral administration, the compounds for use in the method of the invention can be formulated for injection or infusion, for example, intravenous, intra-arterial, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

Additional Dosage Formulations and Drug Delivery Systems

As compared with traditional drug delivery approaches, some controlled release technologies rely upon the modification of both macromolecules and synthetic small molecules to allow them to be actively instead of passively absorbed into the body. For example, XenoPort Inc. utilizes technology that takes existing molecules and re-engineers them to create new chemical entities (unique molecules) that have improved pharmacologic properties to either: 1) lengthen the short half-life of a drug; 2) overcome poor absorption; and/or 3) deal with poor drug distribution to target tissues.

Techniques to lengthen the short half-life of a drug include the use of prodrugs with slow cleavage rates to release drugs over time or that engage transporters in small and large intestines to allow the use of oral sustained delivery systems, as well as drugs that engage active transport systems. Examples of such controlled release formulations, tablets, dosage forms, and drug delivery systems, and that are suitable for use with the present invention, are described in the following published US and PCT patent applications assigned to Xenoport Inc.: US20030158254; US20030158089; US20030017964; US2003130246; WO02100172; WO02100392; WO02100347; WO02100344; WO0242414; WO0228881; WO0228882; WO0244324; WO0232376; WO0228883; and WO0228411. In particular, Xenoport's XP13512 is a transported prodrug of gabapentin that has been engineered to utilize high capacity transport mechanisms located in both the small and large intestine and to rapidly convert to gabapentin once in the body. In contrast to gabapentin itself, XP13512 was shown in preclinical and clinical studies to produce dose proportional blood levels of gabapentin across a broad range of oral doses, and to be absorbed efficiently from the large intestine.

Some other controlled release technologies rely upon methods that promote or enhance gastric retention, such as those developed by Depomed Inc. Because many drugs are best absorbed in the stomach and upper portions of the small intestine, Depomed has developed tablets that swell in the stomach during the postprandial or fed mode so that they are treated like undigested food. These tablets therefore sit safely and neutrally in the stomach for 6, 8, or more hours and deliver drug at a desired rate and time to upper gastrointestinal sites. Specific technologies in this area include: 1) tablets that slowly erode in gastric fluids to deliver drugs at almost a constant rate (particularly useful for highly insoluble drugs); 2) bi-layer tablets that combine drugs with different characteristics into a single table (such as a highly insoluble drug in an erosion layer and a soluble drug in a diffusion layer for sustained release of both); and 3) combination tablets that can either deliver drugs simultaneously or in sequence over a desired period of time (including an initial burst of a fast acting drug followed by slow and sustained delivery of another drug). Examples of such controlled release formulations that are suitable for use with the present invention and that rely upon gastric retention during the postprandial or fed mode, include tablets, dosage forms, and drug delivery systems in the following US patents assigned to Depomed Inc.: U.S. Pat. No. 6,488,962; U.S. Pat. No. 6,451,808; U.S. Pat. No. 6,340,475; U.S. Pat. No. 5,972,389; U.S. Pat. No. 5,582,837; and U.S. Pat. No. 5,007,790. Examples of such controlled release formulations that are suitable for use with the present invention and that rely upon gastric retention during the postprandial or fed mode, include tablets, dosage forms, and drug delivery systems in the following published US and PCT patent applications assigned to Depomed Inc.: US20030147952; US20030104062; US20030104053; US20030104052; US20030091630; US20030044466; US20030039688; US20020051820; WO0335040; WO0335039; WO0156544; WO0132217; WO9855107; WO9747285; and WO9318755.

Other controlled release systems include those developed by ALZA Corporation based upon: 1) osmotic technology for oral delivery; 2) transdermal delivery via patches; 3) liposomal delivery via intravenous injection; 4) osmotic technology for long-term delivery via implants; and 5) depot technology designed to deliver agents for periods of days to a month. ALZA oral delivery systems include those that employ osmosis to provide precise, controlled drug delivery for up to 24 hours for both poorly soluble and highly soluble drugs, as well as those that deliver high drug doses meeting high drug loading requirements. ALZA controlled transdermal delivery systems provide drug delivery through intact skin for as long as one week with a single application to improve drug absorption and deliver constant amounts of drug into the bloodstream over time. ALZA liposomal delivery systems involve lipid nanoparticles that evade recognition by the immune system because of their unique polyethylene glycol (PEG) coating, allowing the precise delivery of drugs to disease-specific areas of the body.

ALZA also has developed osmotically driven systems to enable the continuous delivery of small drugs, peptides, proteins, DNA and other bioactive macromolecules for up to one year for systemic or tissue-specific therapy. Finally, ALZA depot injection therapy is designed to deliver biopharmaceutical agents and small molecules for periods of days to a month using a nonaqueous polymer solution for the stabilization of macromolecules and a unique delivery profile.

Examples of controlled release formulations, tablets, dosage forms, and drug delivery systems that are suitable for use with the present invention are described in the following US patents assigned to ALZA Corporation: U.S. Pat. No. 4,367,741; U.S. Pat. No. 4,402,695; U.S. Pat. No. 4,418,038; U.S. Pat. No. 4,434,153; U.S. Pat. No. 4,439,199; U.S. Pat. No. 4,450,198; U.S. Pat. No. 4,455,142; U.S. Pat. No. 4,455,144; U.S. Pat. No. 4,484,923; U.S. Pat. No. 4,486,193; U.S. Pat. No. 4,489,197; U.S. Pat. No. 4,511,353; U.S. Pat. No. 4,519,801; U.S. Pat. No. 4,526,578; U.S. Pat. No. 4,526,933; U.S. Pat. No. 4,534,757; U.S. Pat. No. 4,553,973; U.S. Pat. No. 4,559,222; U.S. Pat. No. 4,564,364; U.S. Pat. No. 4,578,075; U.S. Pat. No. 4,588,580; U.S. Pat. No. 4,610,686; U.S. Pat. No. 4,612,008; U.S. Pat. No. 4,618,487; U.S. Pat. No. 4,627,851; U.S. Pat. No. 4,629,449; U.S. Pat. No. 4,642,233; U.S. Pat. No. 4,649,043; U.S. Pat. No. 4,650,484; U.S. Pat. No. 4,659,558; U.S. Pat. No. 4,661,105; U.S. Pat. No. 4,662,880; U.S. Pat. No. 4,675,174; U.S. Pat. No. 4,681,583; U.S. Pat. No. 4,684,524; U.S. Pat. No. 4,692,336; U.S. Pat. No. 4,693,895; U.S. Pat. No. 4,704,119; U.S. Pat. No. 4,705,515; U.S. Pat. No. 4,717,566; U.S. Pat. No. 4,721,613; U.S. Pat. No. 4,723,957; U.S. Pat. No. 4,725,272; U.S. Pat. No. 4,728,498; U.S. Pat. No. 4,743,248; U.S. Pat. No. 4,747,847; U.S. Pat. No. 4,751,071; U.S. Pat. No. 4,753,802; U.S. Pat. No. 4,755,180; U.S. Pat. No. 4,756,314; U.S. Pat. No. 4,764,380; U.S. Pat. No. 4,773,907; U.S. Pat. No. 4,777,049; U.S. Pat. No. 4,781,924; U.S. Pat. No. 4,783,337; U.S. Pat. No. 4,786,503; U.S. Pat. No. 4,788,062; U.S. Pat. No. 4,810,502; U.S. Pat. No. 4,812,313; U.S. Pat. No. 4,816,258; U.S. Pat. No. 4,824,675; U.S. Pat. No. 4,834,979; U.S. Pat. No. 4,837,027; U.S. Pat. No. 4,842,867; U.S. Pat. No. 4,846,826; U.S. Pat. No. 4,847,093; U.S. Pat. No. 4,849,226; U.S. Pat. No. 4,851,229; U.S. Pat. No. 4,851,231; U.S. Pat. No. 4,851,232; U.S. Pat. No. 4,853,229; U.S. Pat. No. 4,857,330; U.S. Pat. No. 4,859,470; U.S. Pat. No. 4,863,456; U.S. Pat. No. 4,863,744; U.S. Pat. No. 4,865,598; U.S. Pat. No. 4,867,969; U.S. Pat. No. 4,871,548; U.S. Pat. No. 4,872,873; U.S. Pat. No. 4,874,388; U.S. Pat. No. 4,876,093; U.S. Pat. No. 4,892,778; U.S. Pat. No. 4,902,514; U.S. Pat. No. 4,904,474; U.S. Pat. No. 4,913,903; U.S. Pat. No. 4,915,949; U.S. Pat. No. 4,915,952; U.S. Pat. No. 4,917,895; U.S. Pat. No. 4,931,285; U.S. Pat. No. 4,946,685; U.S. Pat. No. 4,948,592; U.S. Pat. No. 4,954,344; U.S. Pat. No. 4,957,494; U.S. Pat. No. 4,960,416; U.S. Pat. No. 4,961,931; U.S. Pat. No. 4,961,932; U.S. Pat. No. 4,963,141; U.S. Pat. No. 4,966,769; U.S. Pat. No. 4,971,790; U.S. Pat. No. 4,976,966; U.S. Pat. No. 4,986,987; U.S. Pat. No. 5,006,346; U.S. Pat. No. 5,017,381; U.S. Pat. No. 5,019,397; U.S. Pat. No. 5,023,076; U.S. Pat. No. 5,023,088; U.S. Pat. No. 5,024,842; U.S. Pat. No. 5,028,434; U.S. Pat. No. 5,030,454; U.S. Pat. No. 5,071,656; U.S. Pat. No. 5,077,054; U.S. Pat. No. 5,082,668; U.S. Pat. No. 5,104,390; U.S. Pat. No. 5,110,597; U.S. Pat. No. 5,122,128; U.S. Pat. No. 5,125,894; U.S. Pat. No. 5,141,750; U.S. Pat. No. 5,141,752; U.S. Pat. No. 5,156,850; U.S. Pat. No. 5,160,743; U.S. Pat. No. 5,160,744; U.S. Pat. No. 5,169,382; U.S. Pat. No. 5,171,576; U.S. Pat. No. 5,176,665; U.S. Pat. No. 5,185,158; U.S. Pat. No. 5,190,765; U.S. Pat. No. 5,198,223; U.S. Pat. No. 5,198,229; U.S. Pat. No. 5,200,195; U.S. Pat. No. 5,200,196; U.S. Pat. No. 5,204,116; U.S. Pat. No. 5,208,037; U.S. Pat. No. 5,209,746; U.S. Pat. No. 5,221,254; U.S. Pat. No. 5,221,278; U.S. Pat. No. 5,229,133; U.S. Pat. No. 5,232,438; U.S. Pat. No. 5,232,705; U.S. Pat. No. 5,236,689; U.S. Pat. No. 5,236,714; U.S. Pat. No. 5,240,713; U.S. Pat. No. 5,246,710; U.S. Pat. No. 5,246,711; U.S. Pat. No. 5,252,338; U.S. Pat. No. 5,254,349; U.S. Pat. No. 5,266,332; U.S. Pat. No. 5,273,752; U.S. Pat. No. 5,284,660; U.S. Pat. No. 5,286,491; U.S. Pat. No. 5,308,348; U.S. Pat. No. 5,318,558; U.S. Pat. No. 5,320,850; U.S. Pat. No. 5,322,502; U.S. Pat. No. 5,326,571; U.S. Pat. No. 5,330,762; U.S. Pat. No. 5,338,550; U.S. Pat. No. 5,340,590; U.S. Pat. No. 5,342,623; U.S. Pat. No. 5,344,656; U.S. Pat. No. 5,348,746; U.S. Pat. No. 5,358,721; U.S. Pat. No. 5,364,630; U.S. Pat. No. 5,376,377; U.S. Pat. No. 5,391,381; U.S. Pat. No. 5,402,777; U.S. Pat. No. 5,403,275; U.S. Pat. No. 5,411,740; U.S. Pat. No. 5,417,675; U.S. Pat. No. 5,417,676; U.S. Pat. No. 5,417,682; U.S. Pat. No. 5,423,739; U.S. Pat. No. 5,424,289; U.S. Pat. No. 5,431,919; U.S. Pat. No. 5,443,442; U.S. Pat. No. 5,443,459; U.S. Pat. No. 5,443,461; U.S. Pat. No. 5,456,679; U.S. Pat. No. 5,460,826; U.S. Pat. No. 5,462,741; U.S. Pat. No. 5,462,745; U.S. Pat. No. 5,489,281; U.S. Pat. No. 5,499,979; U.S. Pat. No. 5,500,222; U.S. Pat. No. 5,512,293; U.S. Pat. No. 5,512,299; U.S. Pat. No. 5,529,787; U.S. Pat. No. 5,531,736; U.S. Pat. No. 5,532,003; U.S. Pat. No. 5,533,971; U.S. Pat. No. 5,534,263; U.S. Pat. No. 5,540,912; U.S. Pat. No. 5,543,156; U.S. Pat. No. 5,571,525; U.S. Pat. No. 5,573,503; U.S. Pat. No. 5,591,124; U.S. Pat. No. 5,593,695; U.S. Pat. No. 5,595,759; U.S. Pat. No. 5,603,954; U.S. Pat. No. 5,607,696; U.S. Pat. No. 5,609,885; U.S. Pat. No. 5,614,211; U.S. Pat. No. 5,614,578; U.S. Pat. No. 5,620,705; U.S. Pat. No. 5,620,708; U.S. Pat. No. 5,622,530; U.S. Pat. No. 5,622,944; U.S. Pat. No. 5,633,011; U.S. Pat. No. 5,639,477; U.S. Pat. No. 5,660,861; U.S. Pat. No. 5,667,804; U.S. Pat. No. 5,667,805; U.S. Pat. No. 5,674,895; U.S. Pat. No. 5,688,518; U.S. Pat. No. 5,698,224; U.S. Pat. No. 5,702,725; U.S. Pat. No. 5,702,727; U.S. Pat. No. 5,707,663; U.S. Pat. No. 5,713,852; U.S. Pat. No. 5,718,700; U.S. Pat. No. 5,736,580; U.S. Pat. No. 5,770,227; U.S. Pat. No. 5,780,058; U.S. Pat. No. 5,783,213; U.S. Pat. No. 5,785,994; U.S. Pat. No. 5,795,591; U.S. Pat. No. 5,811,465; U.S. Pat. No. 5,817,624; U.S. Pat. No. 5,824,340; U.S. Pat. No. 5,830,501; U.S. Pat. No. 5,830,502; U.S. Pat. No. 5,840,754; U.S. Pat. No. 5,858,407; U.S. Pat. No. 5,861,439; U.S. Pat. No. 5,863,558; U.S. Pat. No. 5,876,750; U.S. Pat. No. 5,883,135; U.S. Pat. No. 5,840,754; U.S. Pat. No. 5,897,878; U.S. Pat. No. 5,904,934; U.S. Pat. No. 5,904,935; U.S. Pat. No. 5,906,832; U.S. Pat. No. 5,912,268; U.S. Pat. No. 5,914,131; U.S. Pat. No. 5,916,582; U.S. Pat. No. 5,932,547; U.S. Pat. No. 5,938,654; U.S. Pat. No. 5,941,844; U.S. Pat. No. 5,955,103; U.S. Pat. No. 5,972,369; U.S. Pat. No. 5,972,370; U.S. Pat. No. 5,972,379; U.S. Pat. No. 5,980,943; U.S. Pat. No. 5,981,489; U.S. Pat. No. 5,983,130; U.S. Pat. No. 5,989,590; U.S. Pat. No. 5,995,869; U.S. Pat. No. 5,997,902; U.S. Pat. No. 6,001,390; U.S. Pat. No. 6,004,309; U.S. Pat. No. 6,004,578; U.S. Pat. No. 6,008,187; U.S. Pat. No. 6,020,000; U.S. Pat. No. 6,034,101; U.S. Pat. No. 6,036,973; U.S. Pat. No. 6,039,977; U.S. Pat. No. 6,057,374; U.S. Pat. No. 6,066,619; U.S. Pat. No. 6,068,850; U.S. Pat. No. 6,077,538; U.S. Pat. No. 6,083,190; U.S. Pat. No. 6,096,339; U.S. Pat. No. 6,106,845; U.S. Pat. No. 6,110,499; U.S. Pat. No. 6,120,798; U.S. Pat. No. 6,120,803; U.S. Pat. No. 6,124,261; U.S. Pat. No. 6,124,355; U.S. Pat. No. 6,130,200; U.S. Pat. No. 6,146,662; U.S. Pat. No. 6,153,678; U.S. Pat. No. 6,174,547; U.S. Pat. No. 6,183,466; U.S. Pat. No. 6,203,817; U.S. Pat. No. 6,210,712; U.S. Pat. No. 6,210,713; U.S. Pat. No. 6,224,907; U.S. Pat. No. 6,235,712; U.S. Pat. No. 6,245,357; U.S. Pat. No. 6,262,115; U.S. Pat. No. 6,264,990; U.S. Pat. No. 6,267,984; U.S. Pat. No. 6,287,598; U.S. Pat. No. 6,289,241; U.S. Pat. No. 6,331,311; U.S. Pat. No. 6,333,050; U.S. Pat. No. 6,342,249; U.S. Pat. No. 6,346,270; U.S. Pat. No. 6,365,183; U.S. Pat. No. 6,368,626; U.S. Pat. No. 6,387,403; U.S. Pat. No. 6,419,952; U.S. Pat. No. 6,440,457; U.S. Pat. No. 6,468,961; U.S. Pat. No. 6,491,683; U.S. Pat. No. 6,512,010; U.S. Pat. No. 6,514,530; U.S. Pat. No. 6,534,089; U.S. Pat. No. 6,544,252; U.S. Pat. No. 6,548,083; U.S. Pat. No. 6,551,613; U.S. Pat. No. 6,572,879; and U.S. Pat. No. 6,596,314.

Other examples of controlled release formulations, tablets, dosage forms, and drug delivery systems that are suitable for use with the present invention are described in the following published US patent application and PCT applications assigned to ALZA Corporation: US20010051183; WO0004886; WO0013663; WO0013674; WO0025753; WO0025790; WO0035419; WO0038650; WO0040218; WO0045790; WO0066126; WO0074650; WO0119337; WO0119352; WO0121211; WO0137815; WO0141742; WO0143721; WO0156543; WO3041684; WO03041685; WO03041757; WO03045352; WO03051341; WO03053400; WO03053401; WO9000416; WO9004965; WO9113613; WO9116884; WO9204011; WO9211843; WO9212692; WO9213521; WO9217239; WO9218102; WO9300071; WO9305843; WO9306819; WO9314813; WO9319739; WO9320127; WO9320134; WO9407562; WO9408572; WO9416699; WO9421262; WO9427587; WO9427589; WO9503823; WO9519174; WO9529665; WO9600065; WO9613248; WO9625922; WO9637202; WO9640049; WO9640050; WO9640139; WO9640364; WO9640365; WO9703634; WO9800158; WO9802169; WO9814168; WO9816250; WO9817315; WO9827962; WO9827963; WO984361 1; WO9907342; WO9912526; WO9912527; WO9918159; WO9929297; WO9929348; WO9932096; WO9932153; WO9948494; WO9956730; WO9958115; and WO9962496.

Another drug delivery technology suitable for use in the present invention is that disclosed by DepoMed, Inc. in U.S. Pat. No. 6,682,759, which discloses a method for manufacturing a pharmaceutical tablet for oral administration combining both immediate-release and prolonged-release modes of drug delivery. The tablet according to the method comprises a prolonged-release drug core and an immediate-release drug coating or layer, which can be insoluble or sparingly soluble in water. The method limits the drug particle diameter in the immediate-release coating or layer to 10 microns or less. The coating or layer is either the particles themselves, applied as an aqueous suspension, or a solid composition that contains the drug particles incorporated in a solid material that disintegrates rapidly in gastric fluid. Andrx Corporation has also developed drug delivery technology suitable for use in the present invention that includes: 1) a pelletized pulsatile delivery system (“PPDS”); 2) a single composition osmotic tablet system (“SCOT”); 3) a solubility modulating hydrogel system (“SMHS”); 4) a delayed pulsatile hydrogel system (“DPHS”); 5) a stabilized pellet delivery system (“SPDS”); 6) a granulated modulating hydrogel system (“GMHS”); 7) a pelletized tablet system (“PELTAB”); 8) a porous tablet system (“PORTAB”); and 9) a stabilized tablet delivery system (“STDS”). PPDS uses pellets that are coated with specific polymers and agents to control the release rate of the microencapsulated drug and is designed for use with drugs that require a pulsed release. SCOT utilizes various osmotic modulating agents as well as polymer coatings to provide a zero-order drug release. SMHS utilizes a hydrogel-based dosage system that avoids the “initial burst effect” commonly observed with other sustained-release hydrogel formulations and that provides for sustained release without the need to use special coatings or structures that add to the cost of manufacturing. DPHS is designed for use with hydrogel matrix products characterized by an initial zero-order drug release followed by a rapid release that is achieved by the blending of selected hydrogel polymers to achieve a delayed pulse. SPDS incorporates a pellet core of drug and protective polymer outer layer, and is designed specifically for unstable drugs, while GMHS incorporates hydrogel and binding polymers with the drug and forms granules that are pressed into tablet form. PELTAB provides controlled release by using a water insoluble polymer to coat discrete drug crystals or pellets to enable them to resist the action of fluids in the gastrointestinal tract, and these coated pellets are then compressed into tablets. PORTAB provides controlled release by incorporating an osmotic core with a continuous polymer coating and a water soluble component that expands the core and creates microporous channels through which drug is released. Finally, STDS includes a dual layer coating technique that avoids the need to use a coating layer to separate the enteric coating layer from the omeprazole core.

Examples of controlled release formulations, tablets, dosage forms, and drug delivery systems that are suitable for use with the present invention are described in the following US patents assigned to Andrx Corporation: U.S. Pat. No. 5,397,574; U.S. Pat. No. 5,419,917; U.S. Pat. No. 5,458,887; U.S. Pat. No. 5,458,888; U.S. Pat. No. 5,472,708; U.S. Pat. No. 5,508,040; U.S. Pat. No. 5,558,879; U.S. Pat. No. 5,567,441; U.S. Pat. No. 5,654,005; U.S. Pat. No. 5,728,402; U.S. Pat. No. 5,736,159; U.S. Pat. No. 5,830,503; U.S. Pat. No. 5,834,023; U.S. Pat. No. 5,837,379; U.S. Pat. No. 5,916,595; U.S. Pat. No. 5,922,352; U.S. Pat. No. 6,099,859; U.S. Pat. No. 6,099,862; U.S. Pat. No. 6,103,263; U.S. Pat. No. 6,106,862; U.S. Pat. No. 6,156,342; U.S. Pat. No. 6,177,102; U.S. Pat. No. 6,197,347; U.S. Pat. No. 6,210,716; U.S. Pat. No. 6,238,703; U.S. Pat. No. 6,270,805; U.S. Pat. No. 6,284,275; U.S. Pat. No. 6,485,748; U.S. Pat. No. 6,495,162; U.S. Pat. No. 6,524,620; U.S. Pat. No. 6,544,556; U.S. Pat. No. 6,589,553; U.S. Pat. No. 6,602,522; and U.S. Pat. No. 6,610,326.

Examples of controlled release formulations, tablets, dosage forms, and drug delivery systems that are suitable for use with the present invention are described in the following published US and PCT patent applications assigned to Andrx Corporation: US20010024659; US20020115718; US20020156066; WO0004883; WO0009091; WO0012097; WO0027370; WO0050010; WO0132161; WO0134123; WO0236077; WO0236100; WO02062299; WO02062824; WO02065991; WO02069888; WO02074285; WO03000177; WO9521607; WO9629992; WO9633700; WO9640080; WO9748386; WO9833488; WO9833489; WO9930692; WO9947125; and WO9961005.

Some other examples of drug delivery approaches focus on non-oral drug delivery, providing parenteral, transmucosal, and topical delivery of proteins, peptides, and small molecules. For example, the Atrigel® drug delivery system marketed by Atrix Laboratories Inc. comprises biodegradable polymers, similar to those used in biodegradable sutures, dissolved in biocompatible carriers. These pharmaceuticals may be blended into a liquid delivery system at the time of manufacturing or, depending upon the product, may be added later by a physician at the time of use. Injection of the liquid product subcutaneously or intramuscularly through a small gauge needle, or placement into accessible tissue sites through a cannula, causes displacement of the carrier with water in the tissue fluids, and a subsequent precipitate to form from the polymer into a solid film or implant. The drug encapsulated within the implant is then released in a controlled manner as the polymer matrix biodegrades over a period ranging from days to months. Examples of such drug delivery systems include Atrix's Eligardo, Atridox®/ Doxirobe®, Atrisorb® FreeFlow™/ Atrisorb®-D FreeFlow, bone growth products, and others as described in the following published US and PCT patent applications assigned to Atrix Laboratories Inc.: U.S. RE37950; U.S. Pat. No. 6,630,155; U.S. Pat. No. 6,566,144; U.S Pat. No. 6,610,252; U.S. Pat. No. 6,565,874; U.S. Pat. No. 6,528,080; U.S. Pat. No. 6,461,631; U.S. Pat. No. 6,395,293; U.S. Pat. No. 6,261,583; U.S. Pat. No 6,143,314; U.S. Pat. No. 6,120,789; U.S. Pat. No. 6,071,530; U.S. Pat. No. 5,990,194; U.S. Pat. No. 5,945,115; U.S. Pat. No. 5,888,533; U.S. Pat. No 5,792,469; U.S. Pat. No. 5,780,044; U.S. Pat. No. 5,759,563; U.S. Pat. No. 5,744,153; U.S. Pat. No. 5,739,176; U.S. Pat. No. 5,736,152; U.S. Pat. No 5,733,950; U.S. Pat. No. 5,702,716; U.S. Pat. No. 5,681,873; U.S. Pat. No. 5,660,849; U.S. Pat. No. 5,599,552; U.S. Pat. No. 5,487,897; U.S. Pat. No. 5,368,859; U.S. Pat. No. 5,340,849; U.S. Pat. No. 5,324,519; U.S. Pat. No. 5,278,202; U.S. Pat. No. 5,278,201; US20020114737, US20030195489; US20030133964; US 20010042317; US20020090398; US20020001608; and US2001042317.

Atrix Laboratories Inc. also markets technology for the non-oral transmucosal delivery of drugs over a time period from minutes to hours. For example, Atrix's BEMA™ (Bioerodible Muco-Adhesive Disc) drug delivery system comprises pre-formed bioerodible discs for local or systemic delivery. Examples of such drug delivery systems include those as described in U.S. Pat. No. 6,245,345.

Other drug delivery systems marketed by Atrix Laboratories Inc. focus on topical drug delivery. For example, SMP™ (Solvent Particle System) allows the topical delivery of highly water-insoluble drugs. This product allows for a controlled amount of a dissolved drug to permeate the epidermal layer of the skin by combining the dissolved drug with a microparticle suspension of the drug. The SMP™ system works in stages whereby: 1) the product is applied to the skin surface-2) the product near follicles concentrates at the skin pore; 3) the drug readily partitions into skin oils; and 4) the drug diffuses throughout the area. By contrast, MCA® (Mucocutaneous Absorption System) is a water-resistant topical gel providing sustained drug delivery. MCA® forms a tenacious film for either wet or dry surfaces where: 1) the product is applied to the skin or mucosal surface; 2) the product forms a tenacious moisture-resistant film; and 3) the adhered film provides sustained release of drug for a period from hours to days. Yet another product, BCPTM (Biocompatible Polymer System) provides a non-cytotoxic gel or liquid that is applied as a protective film for wound healing. Examples of these systems include Orajel®-Ultra Mouth Sore Medicine as well as those as described in the following published U.S. Pat. No. patents and applications assigned to Atrix Laboratories Inc.: U.S. Pat. No. 6,537,565; U.S. Pat. No. 6,432,415; U.S. Pat. No. 6,355,657; U.S. Pat. No. 5,962,006; U.S. Pat. No. 5,725,491; U.S. Pat. No. 5,722,950; U.S. Pat. No. 5,717,030; U.S. Pat. No. 5,707,647; U.S. Pat. No. 5,632,727; and US20010033853.

Additional formulations and compositions available from Teva Pharmaceutical Industries Ltd., Warner Lambert & Co., and Godecke Aktiengesellshaft that include gabapentin and are useful in the present invention include those as described in the following US patents and published US and PCT patent applications: U.S. Pat. No. 6,531,509; U.S. Pat. No. 6,255,526; U.S. Pat. No. 6,054,482; US2003055109; US2002045662; US2002009115; WO 01/97782; WO 01/97612; EP 2001946364; WO 99/59573; and WO 99/59572.

Additional formulations and compositions that include oxybutynin and are useful in the present invention include those as described in the following US patents and published US and PCT patent applications: U.S. Pat. No. 5,834,010; U.S. Pat. No. 5,601,839; and U.S. Pat. No. 5,164,190.

Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams and gels.

Ointments, as is well known in the art of pharmaceutical formulation, are semisolid preparations that are typically based on petrolatum or other petroleum derivatives. The specific ointment base to be used, preferably provides for optimum drug delivery, and, preferably, will provides for other desired characteristics as well, e.g., emolliency or the like. The ointment base is preferably inert, stable, nonirritating and nonsensitizing. As explained in Remington: The Science and Practice of Pharmacy, supra, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy, supra).

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like. During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed above in transmucosal compositions.

Release Profiles

The dual acting SNRI-NMDA antagonists of the present invention can also be administered in pharmaceutical dosage forms with varying release profiles, e.g., delayed release, extended release and pulsatile release. The formulations of the present invention can be, but are not limited to, short-term, rapid-offset, controlled, for example, sustained or extended release, delayed release and pulsatile release formulations.

It would be well within the capability of the skilled artisan to determine the most effective release profile based upon various factors, including, but not limited to, properties of the drug, extent of the illness, etc.

Extended Release Dosage Forms

The terms sustained release or extended release are used interchangeably in their conventional sense to refer to a drug formulation that provides for gradual release of a drug over an extended period of time, and that preferably, although not necessarily, results in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds.

As such, the compounds for use the method of the invention can be administered in the form of microparticles for example, by injection or in the form of wafers or discs by implantation.

The sustained or extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, reservoir and matrix, and is well known and described in the art. The matrix devices are generally prepared by compressing the drug, e.g., the dual acting SNRI-NMDA antagonist of the present invention, with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as camauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form comprising single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

Delayed Release Dosage Forms

The term delayed release is used herein in its conventional sense to refer to a drug formulation that provides for an initial release of the drug after some delay following drug administration and that preferably, although not necessarily, includes a delay of from about 10 minutes up to about 18 hours.

Delayed release formulations are created by coating a solid dosage form with a film of a polymer which is insoluble in the acid environment of the stomach, and soluble in the neutral environment of small intestines.

The delayed release dosage units can be prepared, for example, by coating a drug, e.g., a dual acting SNRI-NMDA antagonist of the present invention, or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit.RTM. (Rohm Pharma; Westerstadt, Germany), including Eudragit.RTM. L30D-55 and L100-55 (soluble at pH 5.5 and above), Eudragit.RTM. L-100 (soluble at pH 6.0 and above), Eudragit.RTM. S (soluble at pH 7.0 and above, as a result of a higher degree of esterification), and Eudragits.RTM. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10% to 50% by weight relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25% to 100% by weight of the polymer in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

The delayed release dosage units may be coated with the delayed release polymer coating using conventional techniques, e.g., using a conventional coating pan, an airless spray technique, fluidized bed coating equipment (with or without a Wurster insert), or the like. For detailed information concerning materials, equipment and processes for preparing tablets and delayed release dosage, reference may be made to Pharmaceutical Dosage Forms: Tablets, eds. Lieberman et al. (New York: Marcel Dekker, Inc., 1989), and to Ansel et al., Pharmaceutical Dosage Forms and Drug Delivery Systems, 6^{th} Ed. (Media, P A: Williams & Wilkins, 1995).

Alternatively, a delayed release tablet may be formulated by dispersing the drug within a matrix of a suitable material such as a hydrophilic polymer or a fatty compound. The hydrophilic polymers may be comprised of polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

Pulsatile Release Dosage Forms

The term pulsatile release is used herein in its conventional sense to refer to a drug formulation that provides release of the drug in such a way as to produce pulsed plasma profiles of the drug after drug administration.

The pharmaceutical dosage forms provide pulsatile delivery of dual acting SNRI-NMDA antagonists of the present invention. By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval may have a duration of approximately 3 hours to 14 hours between the first and second dose. For dosage forms providing a three times daily profile, the nearly drug release-free interval may have a duration of approximately 2 hours to 8 hours between each of the three doses. Additionally, the individual drug-release free intervals within one pulsatile dose may or may not have the same duration.

In one embodiment, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet may release drug substantially immediately following ingestion of the dosage form, while a second tablet may release drug approximately 3 hours to approximately 14 hours following ingestion of the dosage form. For dosage forms mimicking a three times daily dosing profile, a first tablet may release drug substantially immediately following ingestion of the dosage form, a second tablet may release drug approximately 3 hours to approximately 10 hours following ingestion of the dosage form, and the third tablet may release drug approximately 5 hours to approximately 18 hours following ingestion of the dosage form. It is possible that the dosage form includes more than three tablets, e.g., dosage forms housing more than three tablets can be utilized.

Alternatively, each dosage unit in the capsule may comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that may or may not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles may be formulated to provide immediate release, in some preferred embodiments, beads and granules are generally employed to provide delayed release.

In an exemplary formulation, dosage forms mimicking a twice a day dosing profile may include a first group beads, granules or particles which release drug substantially immediately following ingestion of the dosage form, and a second group of beads or granules which release drug approximately 3 hours to approximately 14 hours following ingestion of the dosage form. In another exemplary formulation, dosage forms mimicking a three times daily dosing profile may include a first group of beads, granules or particles which release drug substantially immediately following ingestion of the dosage form, a second group of beads or granules which releases drug approximately 3 hours to approximately 10 hours following ingestion of the dosage form, and a third group of beads, granules or particles which release drug approximately 5 hours to approximately 18 hours following ingestion of the dosage form. Any of the above-mentioned tablets, beads, granules or particles of different drug release profiles (e.g., immediate and delayed release profiles) may be mixed and included in a capsule, tablet or matrix to provide a pulsatile dosage form having the desired release profile.

In another embodiment, the individual dosage units are compacted in a single tablet, and may represent integral but discrete segments thereof (e.g., layers), or may be present as a simple admixture. For example, drug-containing beads, granules or particles with different drug release profiles (e.g., immediate and delayed release profiles) can be compressed together into a single tablet using conventional tableting means.

In a further alternative embodiment, a dosage form is provided that comprises an inner drug-containing core and at least one drug-containing layer surrounding the inner core. An outer layer of this dosage form contains an initial, immediate release dose of the drug. For dosage forms mimicking twice daily dosing, the dosage form has an outer layer that releases drug substantially immediately following oral administration and an inner core having a polymeric-coating that preferably releases the active agent approximately 3 hours to approximately 14 hours following ingestion of the dosage unit. For dosage forms mimicking three times daily dosing, the dosage form has an outer layer that releases drug substantially immediately following oral administration, an inner core that preferably releases drug approximately 5 hours to approximately 18 hours following oral administration and a layer interposed between the inner core and outer layer that preferably releases drug approximately 3 hours to approximately 10 hours following ingestion of the dosage form. The inner core of the dosage form mimicking three times daily a dosing may be formulated as compressed delayed release beads or granules.

Alternatively, for dosage forms mimicking three times daily dosing, the dosage form has an outer layer and an inner layer free of drug. The outer layer releases drug substantially immediately following oral administration, and completely surrounds the inner layer. The inner layer surrounds both the second and third doses and preferably prevents release of these doses for approximately 3 hours to approximately 10 hours following oral administration. Once released, the second dose is immediately available while the third dose is formulated as delayed release beads or granules such that release of the third dose is effected approximately 2 hours to approximately 8 thereafter effectively resulting in release of the third dose approximately 5 hours to approximately 18 hours following ingestion of the dosage form. The second and third doses may be formulated by admixing immediate release and delayed release beads, granules or particles and compressing the admixture to form a second and third dose-containing core followed by polymeric coating to achieve the desired three times daily dosing profile. Additional layers and dosages may be included in the formulation, as necessary. One of ordinary skill in the art would be able to determine the appropriate number of pulses for a given formulation using no more than routine experimentation.

In still another embodiment, a dosage form comprises a coated core-type delivery system wherein the outer layer is comprised of an immediate release dosage unit, such that active agent therein is immediately released following oral administration, an intermediate layer thereunder surrounds a core, and the core is comprised of immediate release beads or granules and delayed release beads or granules, such that the second dose is provided by the immediate release beads or granules and the third dose is provided by the delayed release beads or granules.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily a incorporating a polymeric material; increasing drug particle size; placing the drug within a matrix; and forming complexes of the drug with a suitable complexing agent.

The delayed release dosage units in any of the above embodiments can be prepared by any method for preparing delayed release dosages, for example, by any of the methods described herein.

The immediate release dosage unit of the dosage form—i.e., a tablet, a plurality of drug-containing beads, granules or particles, or an outer layer of a coated core dosage form—preferably contains a therapeutically effective quantity of the active agent with optional conventional pharmaceutical excipients. The term immediate release is used in its conventional sense to refer to a drug formulation that provides for release of the drug immediately after drug administration. The immediate release dosage unit may or may not be coated, and may or may not be admixed with the delayed release dosage unit or units (as in an encapsulated mixture of immediate release drug-containing granules, particles or beads and delayed release drug-containing granules or beads). A preferred method for preparing immediate release tablets (e.g., as incorporated into a capsule) is by compressing a drug-containing blend, e.g., blend of granules, prepared using a direct blend, wet-granulation or dry-granulation process. Immediate release tablets may also be molded rather than compressed, starting with a moist material containing a suitable water-soluble lubricant. However, preferred tablets herein are manufactured using compression rather than molding. A preferred method for forming immediate release drug-containing blend is to mix drug particles directly with one or more excipients such as diluents (or fillers), binders, disintegrants, lubricants, glidants, colorants or the like. As an alternative to direct blending, a drug-containing blend may be prepared by using a wet-granulation or dry-granulation processes. Beads containing the active agent may also be prepared by any one of a number of conventional techniques, typically starting from a fluid dispersion. For example, a typical method for preparing drug-containing beads involves blending the active agent with conventional pharmaceutical excipients such as microcrystalline cellulose, starch, polyvinylpyrrolidone, methylcellulose, talc, metallic stearates, silicone dioxide, or the like. The admixture is used to coat a bead core such as a sugar sphere (or so-called “non-pareil”) having a size of approximately 20 to 60 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

Optional pharmaceutically acceptable excipients present in the drug-containing tablets, beads, granules or particles include, but are not limited to, diluents, binders, lubricants, disintegrants, colorants, stabilizers, surfactants and the like. Diluents, also termed “fillers,” may typically be necessary to increase the bulk of a solid dosage form so that a practical size is provided for compression of tablets or formation of beads and granules. Suitable diluents include, for example, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar. Binders may be used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Lubricants may also be used to facilitate tablet manufacture; examples of suitable lubricants include, for example, magnesium stearate, calcium stearate, stearic acid, glycerol behenate, and polyethylene glycol, talc, and mineral oil. Disintegrants may be used to facilitate dosage form disintegration or “breakup” after administration, and are generally starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp). Stabilizers may be used to inhibit or retard drug decomposition reactions which include, by way or example, oxidative reactions. Surfactants may be anionic, cationic, amphoteric or nonionic surface active agents. Suitable anionic surfactants include, but not limited to those containing carboxylate, sulfonate and sulfate ions. Examples for anionic surfactants are sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but not limited quaternary ammonium compounds such as benzalkonium chloride, benzethonium chloride, cetrimonium bromide, stearyl dimethylbenzyl ammonium chloride, polyoxyethylene (15) and coconut amine. Examples for nonionic surfactants are, but not limited to, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene (8) monolaurate, polysorbates, ii polyoxyethylene (9) octylphenylether, PEG-1000 cetyl ether, polyoxyethylene (3) tridecyl ether, polypropylene glycol (18) butyl ether, Poloxamer 401, stearoyl monoisopropanolamide, and polyoxyethylene (5) hydrogenated tallow amide. Examples for amphoteric surfactants are, but not limited to, sodium N-dodecyl-.beta.-alanine, sodium N-lauryl-.beta.-iminodipropionate, myristoamphoacetate, lauryl betaine and lauryl sulfobetaine. If desired, the tablets, beads granules or particles may also contain minor amount of nontoxic auxiliary substances such as wetting or emulsifying agents, pH buffering agents, preservatives, and the like.

The amount of active agent released in each dose will preferably be a therapeutically effective amount as described herein. Typically, the total amount of active agent in a dosage form is divided evenly between each pulse contained in the dosage form. For dosage forms that mimic a twice a day profile, the active agent in immediate release form generally represents about 30% to 70%, preferably 40% to 60% by weight, of the total active agent in one dosage form, while, correspondingly, the active agent in the delayed release form generally represents about 70% to 30%, preferably 60% to 40% by weight, of the total active agent in one dosage form. Similarly, for dosage forms that mimic three times daily dosing profile, the active agent in the immediate release unit(s) and in each of the two delayed release units represents about 20% to 50%, preferably 25% to 40% by weight, of the total active agent in one dosage form.

All publications mentioned herein are incorporated by reference in their entireties.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

Coadministration

In practicing the methods of the invention, coadministration refers to administration of a dual acting SNRI-NMDA antagonist to treat a symptom associated with a genitourinary disorder with an additional therapeutic agent. Additionally, coadministration refers to administration of a first amount of an SNRI with a second amount of an NMDA antagonist to treat a symptom associated with a genitourinary disorder, optionally with an additional therapeutic agent. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. The compounds are generally administered sufficiently close in time to have the desired therapeutic effect.

Thus, the SNRI can be administered prior to the NMDA antagonist. Additionally or alternatively, the NMDA antagonist can be administered prior to the SNRI. In some embodiments, the second compound may be administered at the same time as the first compound. In other embodiments, the second compound may be administered immediately following the first compound. In still other embodiments, the second compound may be administered 1 minute, 2 minutes, 3 minutes, 4 minutes, 5 minutes, 10 minutes, 15 minutes, 20 minutes . . . 30 minutes after administration of the first compound.

The SNRI and the NMDA antagonist may be administered in the same formulation or different formulations. Additionally, the SNRI and the NMDA antagonist may be administered via the same route or different routes. For example, the SNRI and the NMDA antagonist may both be administered orally in one or more pill formulations. In an alternative example, the SNRI may be administered topically in an ointment formulation and the NMDA antagonist may be administered orally in a pill formulation.

Dosing

The therapeutically effective amount or dose of a dual acting SNRI-NMDA antagonist will depend on the age, sex and weight of the patient, the current medical condition of the patient and the nature of the lower urinary tract disorder being treated. The skilled artisan will be able to determine appropriate dosages depending on these and other factors. As used herein, continuous dosing refers to the chronic administration of a selected active agent.

As used herein, as-needed dosing, also known as “pro re nata” “pm” dosing, and “on demand” dosing or administration is meant the administration of a therapeutically effective dose of the compound(s) at some time prior to commencement of an activity wherein suppression of a lower urinary tract disorder would be desirable.

Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

In a particular embodiment, drug administration or dosing is on an as-needed basis, and does not involve chronic drug administration. With an immediate release dosage form, as-needed administration can involve drug administration immediately prior to commencement of an activity wherein suppression of the symptoms of overactive bladder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity.

A suitable dose of the dual acting SNRI-NMDA antagonist can be in the range of from about 0.001 mg to about 1000 mg per day. In some embodiments, a suitable dose can be in the range of about 0.001 mg to about 500 mg per day, such as from about 0.05 mg to about 500 mg, from about 0.03 mg to about 300 mg, or from about 0.02 mg to about 200 mg per day. In other embodiment, e.g., when a genitourinary disorder is particularly severe, a suitable dose can be in the range of about 500 mg to about 1000 mg per day, such as from about 600 mg to about 900 mg per day, or from about 700 mg to about 800 mg per day. In a particular embodiment, a suitable dose of the dual acting SNRI-NMDA antagonist can be in the range of from about 0.1 mg to about 50 mg per day, such as from about 0.5 mg to about 10 mg per, day such as about 0.5, 1, 2, 3, 4, 5, 6, 7, 8, 9, or 10 mg per day. In preferred embodiments, the dose is between about 10 mg and about 500 mg. In particularly preferred embodiments, the dose is between about 50 mg and about 600 mg per day in the controlled release formulation. Alternatively, the dose of the dual acting SNRI-NMDA antagonist can be greater than or equal to about 0.001 mg, about 0.005 mg, about 0.010 mg, about 0.020 mg, about 0.030 mg, about 0.040 mg, about 0.050 mg, about 0.100 mg, about 0.200 mg, about 0.300 mg, about 0.400 mg, about 0.500 mg, about 1 mg, about 1.5 mg, about 2.0 mg, about 2.5 mg, about 3.0 mg, about 3.5 mg, about 4.0 mg, about 4.5 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. All values in between these values and ranges, e.g., 967 mg, 548 mg, 326 mg, 58.3 mg, 0.775 mg, 0.061 mg, are meant to be encompassed herein. All values in between these values and ranges may also be the upper or lower limits of a range, e.g., a particular dose may include a range of from 178 mg to 847 mg of the dual acting SNRI-NMDA antagonist. Some exemplary doses for bicifadine include 200 mg, 400 mg and 600 mg. An exemplary dose range for milnacipran is 30-200 mg.

The dose per day can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses. Alternatively, a dose of 1 mg per day can be administered four times a day, with dosages at 0.15 mg, 0.25 mg, 0.30 mg and 0.30 mg with about 6 hour intervals between doses.

A suitable dose of the SNRI can be in the range of from about 0.001 mg to about 1000 mg per day, such as from about 0.005 mg to about 750 mg, from about 0.01 mg to about 600 mg, from about 0.02 mg to about 500 mg per day, from about 0.05 mg to about 300 mg per day, from about 0.1 mg to about 200 mg per day, or from about 0.5 mg to about 100 mg per day. Alternatively, the dose of the SNRI can be greater than or equal to about 0.001 mg, about 0.005 mg, about 0.010 mg, about 0.020 mg, about 0.030 mg, about 0.040 mg, about 0.050 mg, about 0.100 mg, about 0.200 mg, about 0.300 mg, about 0.400 mg, about 0.500 mg, about 1 mg, about 1.5 mg, about 2.0 mg, about 2.5 mg, about 3.0 mg, about 3.5 mg, about 4.0 mg, about 4.5 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. All values in between these values and ranges, e.g., 651 mg, 459 mg, 226 mg, 82.5 mg, 0.428 mg, 0.051 mg, are meant to be encompassed herein. All values in between these values and ranges may also be the upper or lower limits of a range, e.g., a particular dose may include a range of from 373 mg to 399 mg of the SNRI. The dose can be administered in a single dosage or in multiple dosages, for example from 1 to 4 or more times per day. When multiple dosages are used, the amount of each dosage can be the same or different.

A suitable dose of the NMDA antagonist can be in the range of from about 0.001 mg to about 1000 mg per day, such as from about 0.005 mg to about 750 mg, from about 0.01 mg to about 600 mg, from about 0.02 mg to about 500 mg per day, from about 0.05 mg to about 300 mg per day, from about 0.1 mg to about 200 mg per day, or from about 0.5 mg to about 100 mg per day. Alternatively, the dose of the NMDA antagonist may be greater than or equal to about 0.001 mg, about 0.005 mg, about 0.010 mg, about 0.020 mg, about 0.030 mg, about 0.040 mg, about 0.050 mg, about 0.100 mg, about 0.200 mg, about 0.300 mg, about 0.400 mg, about 0.500 mg, about 1 mg, about 1.5 mg, about 2.0 mg, about 2.5 mg, about 3.0 mg, about 3.5 mg, about 4.0 mg, about 4.5 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. All values in between these values and ranges, e.g., 954 mg, 521 mg, 81.3 mg, 0.451 mg, 0.0825 mg, are meant to be encompassed herein. All values in between these values and ranges may also be the upper or lower limits of a range, e.g., a particular dose may include a range of from 286 mg to 322 mg of the NMDA antagonist. The dose can be administered in a single dosage or in multiple dosages, for example from 1 to 4 or more times per day. When multiple dosages are used, the amount of each dosage can be the same or different.

It is understood that the amount of compound dosed per day can be administered multiple times per day, every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc. Additionally or alternatively, the dosages may vary from day to day. For example, in a daily dosage regime, 6 mg may be administered on Monday, 8 mg on Tuesday, 10 mg on Wednesday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

For the dual acting SNRI-NMDA antagonist, each dosage can typically contain from about 0.001 mg to about 1000 mg, such as from about 0.05 mg to about 500 mg, for example, from about 0.03 mg to about 300 mg, such as about 0.02 mg to about 200 mg of the active ingredient. That is, suitable dosages contain at least 5% active ingredient. In some embodiments, suitable dosages contain at least 10%, 25%, 50% 75% . . . 100% active ingredient.

When the method of treatment comprises coadministration of an SNRI and a NMDA antagonist, each dose can typically contain from about 0.001 mg to about 1000 mg, such as from about 0.05 mg to about 500 mg, for example, from about 0.03 mg to about 300 mg, such as about 0.02 mg to about to about 200 mg of the NMDA antagonist and typically can contain from about 0.001 mg to about 500 mg, such as from about 0.01 mg to about 100 mg, for example, from about 0.05 mg to about 50 mg, such as about 0.5 mg to about 25 mg of the SNRI. That is, suitable dosages contain at least 1% SNRI. In some embodiments, suitable dosages contain at least 5%, 10%, 20%, 30%, 40%, 50%, 60%, 75% . . . 99% SNRI. Suitable dosages also contain at least 1% NMDA antagonist. In some embodiments, suitable dosages contain at least 5%, 10%, 20%, 30%, 40%, 50%, 60%, 75% . . . 99% NMDA antagonist.

The invention further includes a kit for treating a genitourinary disorder in a subject in need of treatment. The kit generally includes a dual acting SNRI-NMDA antagonist, packaged with an instructional insert for using the dual acting SNRI-NMDA antagonist for the treatment of the genitourinary disorder. In one embodiment, the dual acting SNRI-NMDA antagonist may include a first amount of an SNRI and a second amount of an NMDA antagonist. In another embodiment, the dual acting SNRI-NMDA antagonist includes one agent having both SNRI activity and NMDA antagonist activity.

Alternatively, the kit may include an SNRI packaged with an instructional insert for using the SNRI together with an NMDA antagonist for the treatment of a genitourinary disorder. The kit may otherwise include an NMDA antagonist packaged with an instructional insert for using the NMDA antagonist together with an SNRI for the treatment of a genitourinary disorder.

In other embodiments of the kits, the instructional insert futher includes instructions for administration with an additional therapeutic agent as described herein.

In one embodiment, the instructional insert includes instructions for administration with an additional therapeutic agent based upon the functional relationship between the agents. For example, an SNRI may be packaged with an instructional insert which details the administration of the SNRI with an NMDA antagonist such that they work synergistically. In other examples, an NMDA antagonist may packaged with an instructional insert which details the administration of the NMDA antagonist with an SNRI such that they work additively, and/or such that they do not cause an increase in heart rate or arterial pressure. In still other examples, the dual acting SNRI-NMDA antagonist may be packaged with an instructional insert which details the administration of the dual acting SNRI-NMDA antagonist with a carrier or other therapeutic agent such that their activities do not interfere with each other.

It is understood that in practicing the method or using a kit of the present invention that administration encompasses administration by different individuals (e.g., the subject, physicians or other medical professionals) administering the same or different compounds.

As used herein, the term pharmaceutically acceptable salt refers to a salt of the administered compounds prepared from pharmaceutically acceptable non-toxic acids including inorganic acids, organic acids, solvates, hydrates, or clathrates thereof.

Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

It is understood that dual acting SNRI-NMDA antagonists, SNRIs or NMDA antagonists can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

The invention also relates to a method of processing a claim under a health insurance policy submitted by a claimant seeking reimbursement for costs associated with the treatment of a genitourinary disorder as described herein.

In one embodiment, the method for processing a claim under a health insurance policy submitted by a claimant seeking reimbursement for costs associated with treatment of at least one symptom associated with a genitourinary disorder is accomplished using a therapeutically effective amount of a dual acting SNRI-NMDA antagonist. The method includes reviewing the claim, determining whether the treatment is reimbursable under the insurance policy, and processing the claim to provide partial or complete reimbursement of the costs.

In some embodiments, the dual acting SNRI-NMDA antagonist includes a first amount of an SNRI and a second amount of an NMDA antagonist. The first amount and the second amount may each be a therapeutically effective amount. Alternatively, the first amount and the second amount together form a therapeutically effective amount. In other embodiments, the dual acting SNRI-NMDA antagonist includes one agent having both SNRI activity and NMDA antagonist activity.

In one embodiment, the genitourinary disorder can be overactive bladder, overactive bladder with sphincter dysfunction, stress urinary incontinence, Fowler's Syndrome, outlet obstruction, outlet insufficiency, pelvic hypersensitivity, sphincteric spasticity, detrusor hyperreflexia (neurogenic bladder), detrusor instability, benign prostatic hyperplasia (BPH), urethral stricture disease, tumors, interstitial (cell) cystitis, chronic pelvic pain syndrome, prostatodynia, prostatis, vulvodynia, vulvar vestibulitis, urethritis, and orchidalgia

In a preferred embodiment, the genitourinary disorder is characterized by bladder-sphincter dyssynergia. In another preferred embodiment, the symptom associated with a genitourinary disorder is overactive bladder, overactive bladder with sphincter dysfunction, stress urinary incontinence, Fowler's Syndrome, chronic pelvic pain syndrome, prostatitis, prostatodynia, vulvodynia, vestibulitis, and benign prostatic hyperplasia.

In one aspect of the invention, the dual acting SNRI-NMDA antagonist is a bicifadine compound, a milnacipran compound or a delucemine compound.

Exemplification

The present invention will now be illustrated by the following Examples, which are not intended to be limiting in any way.

Treatment of Overactive Bladder Using a Dual Acting SNRI-NMDA Antagonist

Drugs and Preparation:

Bicifadine was obtained from Evotec. For intravenous administration of bicifadine, the drug was dissolved in saline at 10 mg/ml. Serial dilutions were performed using saline for final concentrations of 0.1, 0.3, 1, 3 and 10 mg/ml. Vehicle was given as saline.

Milnacipran was obtained from Taizhou Dongdong Pharmachem Co. Ltd. (China). For intravenous administration of milnacipran, the drug was also dissolved in saline at 10 mg/ml.

### EXAMPLE 1

### Acute Rat Models: Dilute Acetic Acid Model and Protamine Sulfate/Physiological Urinary Potassium Model

The acute models described below provide methods for evaluating active agents in the treatment of overactive bladder. Briefly, the models provide a method for reducing the bladder capacity of test animals by infusing either protamine sulfate and potassium chloride (See, Chuang, Y. C. et al., Urology 61(3): 664-670 (2003)) or dilute acetic acid (See, Thor, K. and Katofiasc., M., 1995, J. Pharmacol. Exptl. Ther., 274:1014-1024; Sasaki, K. et al, J. Urol. 168(3) 1259-1264 (2002)) into the bladder.

The infusates cause irritation of the bladder and a reduction in bladder capacity by selectively activating bladder afferent fibers, such as C-fiber afferents. Following irritation of the bladder, an active agent (drug) can be administered and the ability of the active agent to reverse (partially or totally) the reduction in bladder capacity resulting from the irritation, can be determined. Substances which reverse the reduction in bladder capacity can be used in the treatment of overactive bladder.

Animal Preparation:

Female rats (250-275 g) were anesthetized with urethane (1.2 g/kg) and a saline-filled jugular catheter (PE-50) was inserted for intravenous drug administration and a heparinized (100 units/ml) saline-filled carotid catheter (PE-50) was inserted for blood pressure monitoring. Via a midline abdominal incision from xyphoid to navel, a PE-50 catheter was inserted into the bladder dome for bladder filling and pressure recording. The abdominal cavity was moistened with saline and closed by covering with a thin plastic sheet in order to maintain access to the bladder for filling cystometry emptying purposes. Fine silver or stainless steel wire electrodes were inserted into the external urethral sphincter (EUS) percutaneously for electromyography (EMG).

Dilute Acetic Acid Model:

Saline and all subsequent infusates were continuously infused at a rate of about 0.055 ml/min via the bladder filling catheter for 30-60 minutes to obtain a baseline of lower urinary tract activity (continuous cystometry; CMG). Bladder pressure traces act as direct measures of bladder and urethral outlet activity, and EUS-EMG phasic firing and voiding act as indirect measures of lower urinary tract activity during continuous transvesical cystometry. Following the control period, a 0.25% acetic acid solution in saline (AA) was infused into the bladder to induce bladder irritation. Following 30 minutes of AA infusion, 3 vehicle injections will be made at 20 minute intervals to determine vehicle effects, if any. Subsequently, increasing doses of bicifidine (1, 3, and 10 mg/kg) administered intravenously at 30 minute intervals in order to construct a cumulative dose-response relationship (n=6). At the end of the control saline cystometry period, the third vehicle injection, and 20 minutes following each subsequent treatment, the infusion pump will be stopped, the bladder will be emptied by fluid withdrawal via the infusion catheter and a single filling cystometrogram will be performed at the same flow rate in order to determine changes in bladder capacity caused by the irritation protocol and subsequent drug administration. In this acute model, C-fiber afferent pathways within the bladder are selectively activated.

Protamine Sulfate/Physiological Urinary Potassium Model (Prospective):

Saline and all subsequent infusates will continuously be infused at a rate of about 0.055 ml/min via the bladder filling catheter for about 30-60 minutes to obtain a baseline of lower urinary tract activity (continuous cystometry; CMG). Bladder pressure traces act as direct measures of bladder and urethral outlet activity, and EUS-EMG phasic firing and voiding act as indirect measures of lower urinary tract activity during continuous transvesical cystometry. Following the control period, a 10 mg/mL protamine sulfate (PS) in saline solution will be infused for about 30 minutes in order to permeabilize the urothelial diffusion barrier. After PS treatment, the infusate will be switched to 300 mM KCl in saline to induce bladder irritation. Once a stable level of lower urinary tract hyperactivity is established (20-30 minutes), 3 vehicle injections will be made at about 30 minute intervals to assess the effects of the vehicle.

Subsequently, increasing doses of a selected active agent will be administered intravenously at about 30 minute intervals in order to construct a cumulative dose-response relationship. At the end of the control saline cystometry period, the third vehicle injection, and 20 minutes following each subsequent treatment, the infusion pump will be stopped, the bladder will be emptied by fluid withdrawal via the infusion catheter and a single filling cystometrogram will be performed at the same flow rate in order to determine changes in bladder capacity caused by the irritation protocol and subsequent drug administration. This model acutely activates bladder afferent fibers, including, C-fiber afferents.

Data Analysis

Data for bicifadine were analyzed by non-parametric ANOVA for repeated measures (Friedman Test) with Dunn's Multiple Comparison test for cumulative dose-response studies. All comparisons were made from the last vehicle measurement. P<0.050 was considered significant. Additionally, % voiding efficiency was calculated using the following formula: (Continuous Cystometry Intermicturition Interval/(Single Cystometrogram Bladder Capacity))*100 for bicifadine.

Results

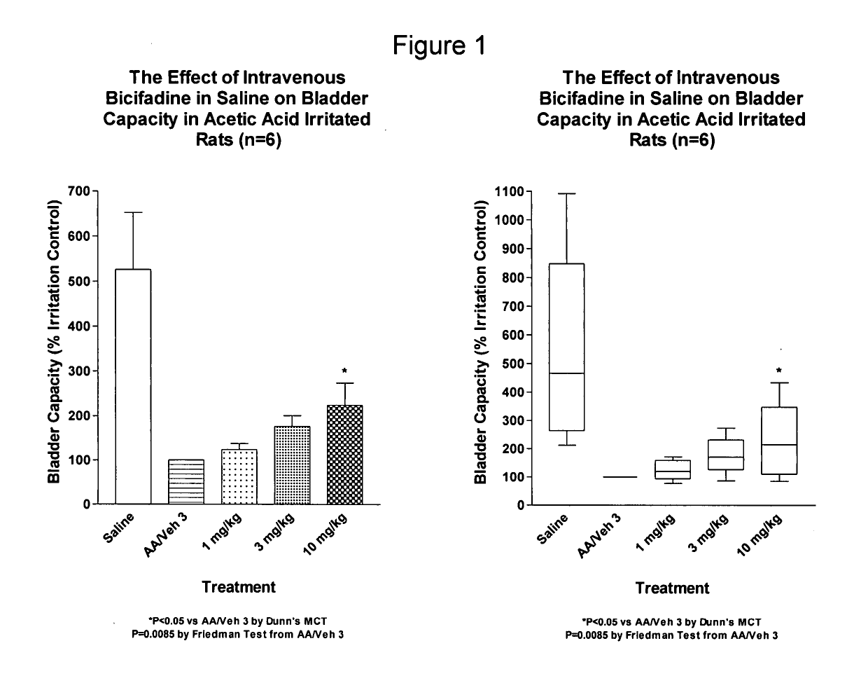

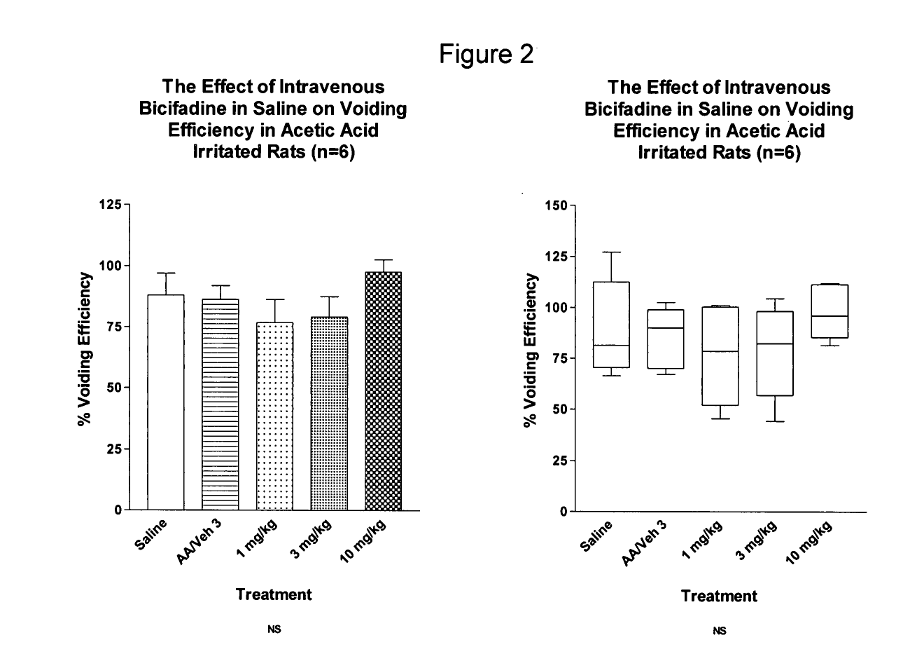

The effect of cumulative addition of increasing doses of bicifadine on bladder capacity and voiding efficiency are depicted in FIGS. 1 and 2. Bladder capacity data for each animal were normalized to “% Irritation Control”, the value of the measure taken after the third vehicle during acetic acid irritation, and this index was used as the measure of efficacy. Data for both bladder capacity and voiding efficiency are presented as means (left graph) as well as a box plot (right graph), which shows the median, the upper and lower quartiles and the range of data collected. Note that all concentrations of bicifadine had an effect on the bladder capacity under conditions of acetic acid infusion compared to vehicle. Additionally, voiding efficiency is not effected by increasing doses of bicifadine.

Conclusions

The data indicate that bicifadine increased bladder capacity significantly from 1-10 mg/kg producing an approximate 100% increase in bladder capacity (FIG. 1) under conditions of acetic acid infusion compared to vehicle. Additionally, voiding efficiency was basically unaffected under conditions of acetic acid infusion compared to vehicle. It is also noteworthy, as shown in the box plots, that the medians track the means.

The data described above is also supportive of the same described efficacy in humans. That is, it is believed that the administration of a suitable dose of a dual acting SNRI-NMDA antagonist, e.g., bicifadine or milnacipran, would increase bladder capacity while maintaining voiding efficency. Furthermore, it is expected based upon the presented data that the administration of a suitable dose of a dual acting SNRI-NMDA antagonist will treat or alleviate at least one of the following disorders: frequency, urgency, nocturia, incontinence, (e.g., urge incontinence) detrusor muscle overactivity/hyperactivity, and other genitourinary disorders described herein.

### EXAMPLE 2

### A Cute Cat Models: Dilute Acetic Acid Model

The ability of bicifadine and milnacipran, exemplary dual acting SNRI-NMDA antagonists, to reverse the reduction in bladder capacity observed following continuous infusion of dilute acetic acid in a cat model was tested. The cat model is a commonly used model for overactive bladder (Thor and Katofiasc, 1995, J. Pharmacol. Exptl. Ther. 274: 1014-24).

Animal Preparation:

Female cats (2.2-3 kg B W Harlan) were anesthetized with 4% isoflurane for induction followed by intravenous administration of α-chloralose (68-75 mg/kg). An i.v. catheter was inserted in the radial vein for drug administration. A heparinized (100 units/ml) saline-filled catheter (PE-90) was inserted into the carotid artery to monitor blood pressure. A trachea tube was place to monitor respiration. Via a midline lower abdominal incision, a modified 16 gauge i.v. catheter was inserted into the bladder dome for bladder filling and pressure recording. The abdominal cavity was moistened with saline and closed by covering with a thin plastic sheet in order to maintain access to the bladder for emptying purposes. Fine silver or stainless steel wire electrodes were inserted intraurethrally into the external urethral sphincter (EUS) for electromyography (EMG).

Intravenous Drug Administration:

Saline was continuously infused at a rate of 0.5-1 ml(s)/min via the bladder catheter for 60 minutes to obtain baseline of lower urinary tract activity (continuous cystometry; CMG). Following the control period, a 0.5% acetic acid (AA) solution in saline was infused into the bladder at the same flow rate to induce bladder irritation.

Following 30 minutes of AA infusion, in one example, 1-2 vehicle injections were made at 30 minute intervals to determine vehicle effects, if any. Subsequently, increasing doses of bicifadine (0.1, 0.3, 1, 3 and 10 mg/kg) were administered intravenously at 30-60 minute intervals in order to construct a cumulative dose-response relationship (n=6). At the end of the control saline cystometry period, the vehicle, and 15 minutes following each subsequent treatment, the infusion pump was stopped, the bladder was emptied by fluid withdrawal via the infusion catheter and a single filling cystometrogram was performed at the same flow rate in order to determine changes in bladder capacity caused by the irritation protocol and subsequent drug administration.

In a second example, subsequent to AA infusion, a vehicle injection was made to ensure that there were no vehicle effects. 10 mg/kg of milnacipran was then administered intravenously. The external urethral EMG activity, bladder pressure, and volume voided was measured during saline infusion, during AA infusion, and subsequent to milnacipran administration.

Data Analysis

Data for bicifadine were analyzed by non-parametric ANOVA for repeated measures (Friedman Test) with Dunn's Multiple Comparison test for cumulative dose-response studies. All comparisons were made from the last vehicle measurement. P<0.050 was considered significant.

Results

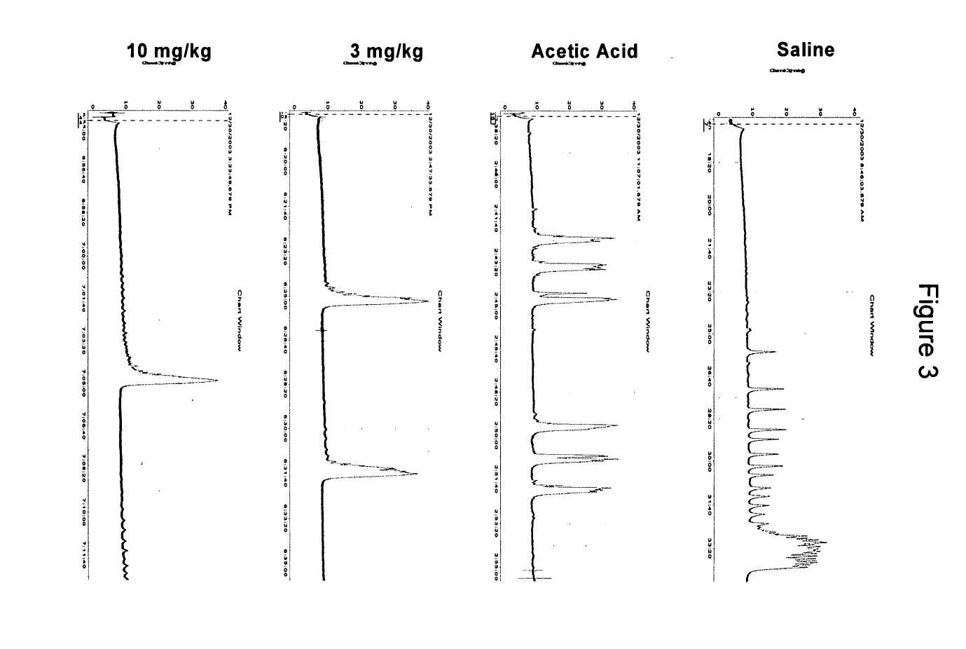

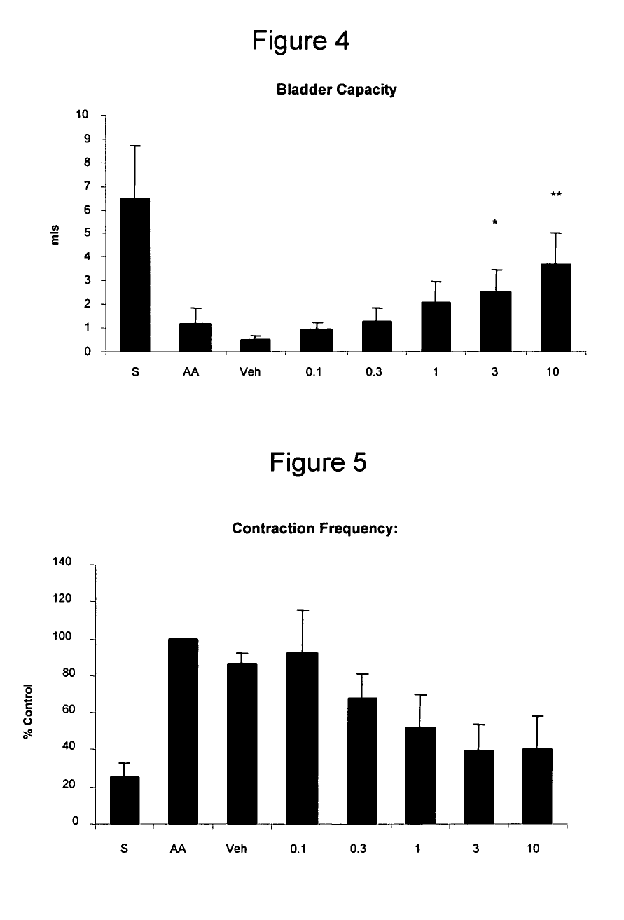

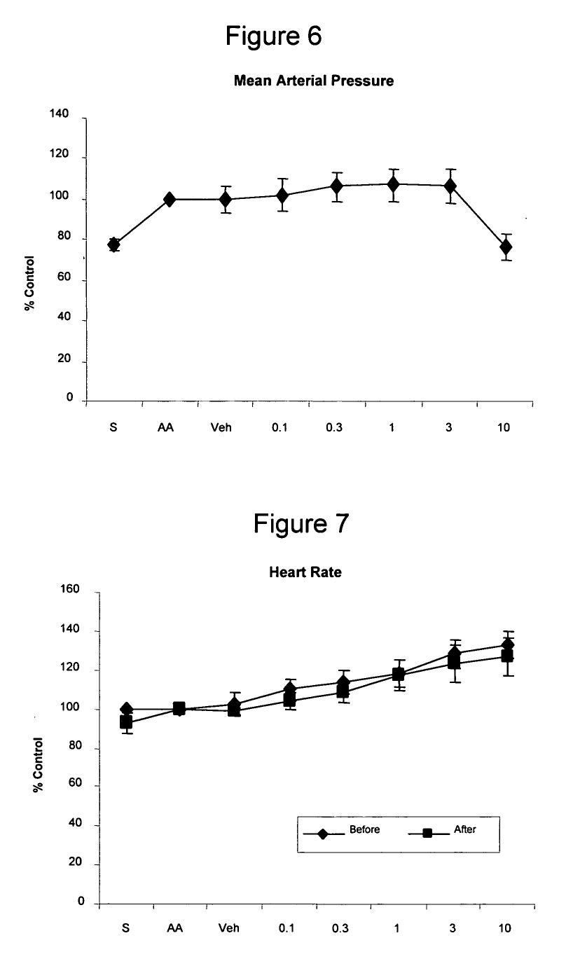

Intravenous bicifadine produced a significant increase in bladder capacity, and also reduced bladder overactivity (i.e., contraction frequency) during continuous cystometry, in the acetic acid bladder irritation model (FIGS. 3, 4 and 5). No significant decrease in arterial pressure and no significant changes in heart rate (FIGS. 6 and 7) were noted. Increases in sphincter EMG during bladder filling were seen in two of the six experiments. In these two experiments, the sphincter EMG activity was inhibited during micturition contractions, indicating maintenance of bladder-sphincter synergy.

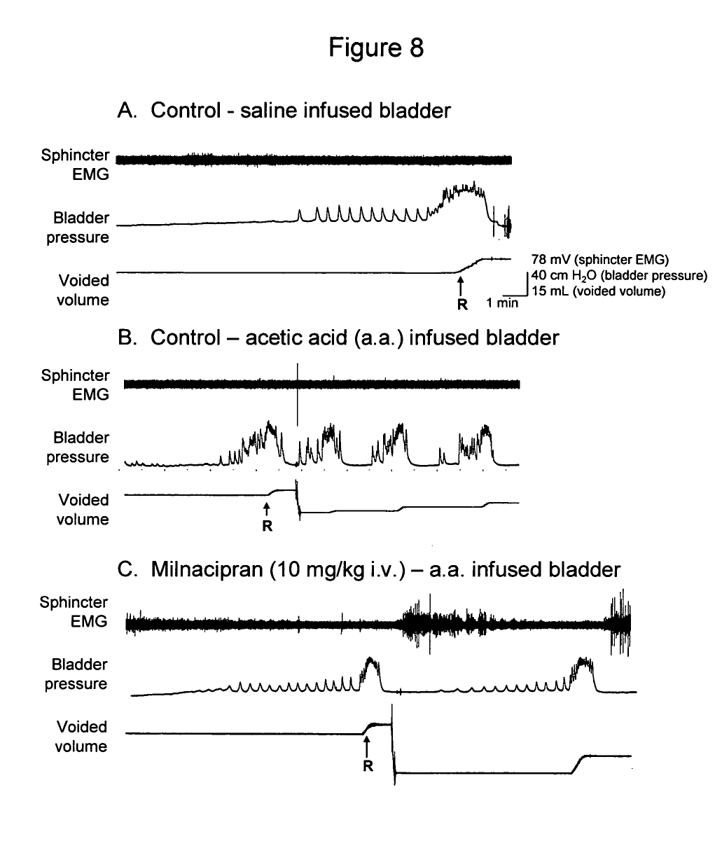

Intravenous milnacipran also produced a significant increase in bladder capacity during acetic acid infusion (FIG. 8). There was also an unexpected increase in sphincter EMG activity, which remained at baseline levels during saline and AA infusion, when milnacipran was administered. The increase in sphincter EMG activity after drug administration, measured as Root Mean Square, was 19.4 millivolts during the period of bladder filling and 82.5 millivolts during the period following the bladder contraction.

Conclusions

The data indicate that bicifadine increased bladder capacity significantly from 1-10 mg/kg producing a 3 fold increase in bladder capacity (FIG. 5) under conditions of acetic acid infusion compared to vehicle. Contraction frequency showed a dose dependent decrease from 1-10 mg/kg but this was not significant. Additionally, voiding efficiency was also increased from 1-10 mg/kg, producing an approximate 10% increase in voiding efficiency under conditions of acetic acid infusion compared to vehicle. No significant changes were noted on mean arterial pressure or heart rate.

The data also indicate that milnacipran produced an approximate 2 fold increase in bladder capacity under conditions of acetic acid infusion as compared to a control, and an unexpected increase in sphincter EMG activity upon administration.

The data described above is also supportive of the same described efficacy in humans. That is, it is believed that the administration of a suitable dose of a dual acting SNRI-NMDA antagonist, e.g., bicifadine or milnacipran, would increase bladder capacity, decrease contraction frequency, and maintain bladder-sphincter synergy without greatly influencing arterial pressure or heart rate. Furthermore, it is expected based upon the presented data that the administration of a suitable dose of a dual acting SNRI-NMDA antagonist will treat or alleviate at least one of the following disorders: frequency, urgency, nocturia, incontinence, (e.g., urge incontinence) detrusor muscle overactivity/hyperactivity, and other genitourinary disorders described herein.

### PROSPECTIVE EXAMPLE

Chronic Rat Model: Chronic Spinal Cord Injury Model

The following is a model of neurogenic bladder, in which C-fiber afferents are chronically activated as a result of spinal cord injury (See, Yoshiyama, M. et al., Urology 54(5): 929-933 (1999)). Following spinal cord injury an active agent (drug) can be administered and the ability of the active agent to reverse (partially or totally) the reduction in bladder capacity resulting from spinal cord injury can be determined. Substances which reverse the reduction in bladder capacity can be used in the treatment of overactive bladder, for example, neurogenic bladder.

Animal Preparation for Chronic Model:

Female rats (250-275 g) will be anesthetized with isofluorane (4%) and a laminectomy will be performed at the T9-10 spinal level. The spinal cord will be transected and the intervening space filled with Gelfoam. The overlying muscle layers and skin will sequentially be closed with suture, and the animals will be treated with antibiotic (100 mg/kg ampicillin s.c.). Residual urine will be expressed prior to returning the animals to their home cages, and thereafter 3 times daily until terminal experimentation four weeks later. On the day of the experiment, the animals will be anesthetized with isofluorane (4%) and a jugular catheter (PEIO) will be inserted for access to the systemic circulation and tunneled subcutaneously to exit through the midscapular region. Via a midline abdominal incision, a PE50 catheter with a fire-flared tip will be inserted into the dome of the bladder through a small cystotomy and secured by ligation for bladder filling and pressure recording. Small diameter (75 μm) stainless steel wires will be inserted percutaneously into the external urethral sphincter (EUS) for electromyography (EMG).

The abdominal wall and the overlying skin of the neck and abdomen will be closed with suture and the animal will be mounted in a Ballman-type restraint cage. A water bottle will be positioned within easy reach of the animal's mouth for ad libitum access to water. The bladder catheter will be hooked up to the perfusion pump and pressure transducer, and the EUS-EMG electrodes to their amplifier. Following a 30 minute recovery from anesthesia and acclimatization, normal saline will be infused at a constant rate (0.100-0.150 ml/min) for control cystometric recording.

Chronic Spinal Cord Injury Model:

Following a 60-90 minute control period of normal saline infusion (0.100-0.150 ml/min) to collect baseline continuous open cystometric data, the pump will be turned off, the bladder will be emptied, the pump turned back on, and bladder capacity will be estimated by a filling cystometrogram. At 3×20-30 minute intervals, vehicle will be administered intravenously in order to ascertain vehicle effects on bladder activity. Following the third vehicle control, bladder capacity will again be estimated as described above. Subsequently, a cumulative dose-response will be performed with the agent of choice. Bladder capacity will be measured 20 minutes following each dose. This is a model of neurogenic bladder, in which C-fiber afferents are chronically activated.

While this invention has been particularly shown and described with references to preferred embodiments thereof, it will be understood by those skilled in the art that various changes in form and details may be made therein without departing from the scope of the invention encompassed by the appended claims.

1. A method of treating a genitourinary disorder in a subject in need of treatment, comprising administering to the subject a therapeutically effective amount of a dual acting SNRI-NMDA antagonist, such that the genitourinary disorder is treated:

2. The method of claim 1, wherein the dual acting SNRI-NMDA antagonist comprises:
a first amount of an SNRI; and
a second amount of an NMDA antagonist.

3. The method of claim 2, wherein the first amount is a therapeutically effective amount and the second amount is a therapeutically effective amount.

4. The method of claim 2, wherein the first amount and the second amount together form a therapeutically effective amount.

5. The method of claim 1, wherein the dual acting SNRI-NMDA antagonist comprises:
one agent having both SNRI activity and NMDA antagonist activity.

6. The method of claim 1, wherein the dual acting SNRI-NMDA antagonist is a compound of Formula I: 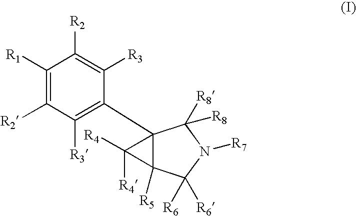
wherein:
R₁, R₂, R₂′, R₃, R₃′, R₄, R₄′, R₅, R₆, R₆′, R₇, R₈, and R₈′ are each independently selected from the group consisting of H, alkyl, aryl, alkenyl, alkynyl, alkoxy, alkoxyalkyl, cycloalkyl, acyl, aroyl, carboxyl, carbonyl, amino, alkylamino, dialkylamino, nitro, halogen, hydroxyl, amido, acetamido, or trifluoromethyl; or
R₄ and R₄′ together form ═O, ═S, ═NH or ═CH₂; and/or
R₆ and R₆′ together form ═O, ═S, ═NH or ═CH₂; and/or
R₈ and R₈′ together form ═O, ═S, ═NH or ═CH₂
or pharmaceutically acceptable salts thereof.

7. The method of claim 6, wherein:
R₁, R₂, R₂′, R₃ and R₃′ are each independently selected from the group consisting of H, alkyl, aryl, alkoxy, alkoxyalkyl, cycloalkyl, amino, nitro, acetamido, hydroxyl, trifluoromethyl and halogen;
R₄, R₄′, R₅, R R₆′, R₈, and R₈′ are each independently selected from the group consisting of H, alkyl, aryl, alkoxy, alkoxyalkyl, cycloalkyl, halogen and hydroxyl; or
R₆ and R₆′ together form ═O, ═S, ═NH or ═CH₂; and/or
R₈ and R₈′ together form ═O, ═S, ═NH or ═CH₂; and
R₇ is selected from the group consisting of H, alkyl, aryl, alkoxy, alkoxyalkyl, cycloalkyl, acyl, carboxyl and carbonyl.

8. The method of claim 6, wherein:
R₁, R₂, R₂′, R₃ and R₃′ are each independently selected from the group consisting of H, C₁-C₆ alkyl, C₁-C₆ alkoxy and halogen;
R₄, R₄′, R₅, R₆, R₆′, R₈, and R₈′ are each independently selected from the group consisting of H, C₁-C₆ alkyl, halogen and hydroxyl; or
R₆ and R₆′ together form ═O; and/or
R₈ and R₈′ together form ═O; and
R₇ is selected from the group consisting of H or C₁-C₆ alkyl optionally substituted with aryl or substituted aryl.

9. The method of claim 6, wherein at least one of R₁, R₂, R₂′, R₃ and R₃′ is other than hydrogen.

10. The method of claim 6, wherein R₁ and R₂′, are not both chlorine when R₂, R₃, R₃′, R₄, R₄′, R₅, R₆, R₆′, R₇, R₈, and R₈′ are hydrogen.

11. The method of claim 6, wherein the dual acting SNRI-NMDA antagonist is a single enantiomer.

12. The method of claim 1, wherein the dual acting SNRI-NMDA antagonist is at least one member selected from the group consisting of compounds represented by the following structural formulas:

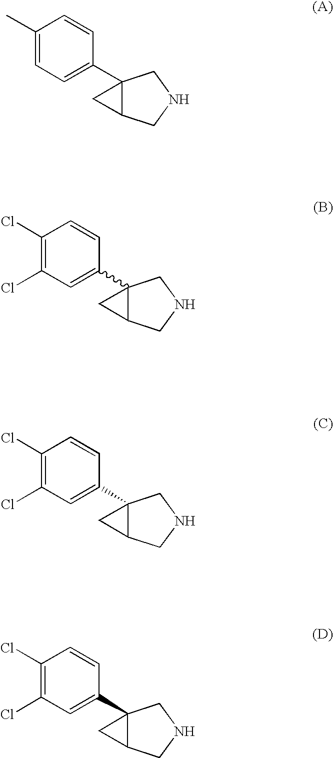

13. The method of claim 1, wherein the dual acting SNRI-NMDA antagonist is a compound of Formula II: 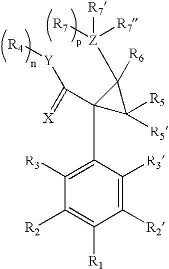
wherein:
X is selected from the group consisting of O, S and NR;
Y is selected from the group consisting of O, S, and NR₄′;
n and p are each independently 0 or 1;
optionally R₇ and R₄ together form a direct bond between Y and Z;
R, R₁, R₂, R₂′, R₃, R₃′, R₅, R₅′, R₆, R₇, and R₇′ are each independently H, alkyl, aryl, heteroaryl, arylalkyl, alkenyl, alkynyl, alkoxy, alkoxyalkyl, aryloxy, arylalkyloxy, cycloalkyl, acyl, aroyl, carboxyl, carbonyl, amino, alkylamino, dialkylamino, arylamino, arylakylamino, nitro, halogen, hydroxyl, amido, acetamido, or trifluoromethyl, cyano, thio, alkylthio, arylthio, arylakylthio, azido, alkylseleno, formyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₉;
R₇″ is selected from the group consisting of H, NR₈R₈′, OR₈, and SR₈;
R₉ is selected from the group consisting of aryl, cycloalkyl, heterocyclyl or polycyclyl;
m is an integer from 0 to 8;
R₄, R₄′, R₈, and R₈′ are each independently selected from the group consisting of H, alkyl, aryl, aralkyl, alkenyl, alkynyl, alkoxyalkyl, cycloalkyl, acyl, aroyl, carboxyl, and carbonyl;
optionally R₄ and R₄′ together form a heterocyclic ring;
optionally R₈ and R₈′ together form a heterocyclic ring; and
optionally R₇ and R₇′ together form ═O, ═S, ═NH or ═CH₂;
or pharmaceutically acceptable salts thereof.

14. The method of claim 13, wherein X is O.

15. The method of claim 13, wherein Y is selected from the group consisting of O and NF₄′.

16. The method of claim 13, wherein n and p are each 1.

17. The method of claim 13, wherein R₁, R₂, R₂′, R₃, R₃′, are each independently selected from the group consisting of H, alkyl, alkoxy, amino, nitro, halogen, or hydroxyl.

18. The method of claim 13, wherein R₄ and R₄′ are each independently selected from the group consisting of H, alkyl, aryl, aralkyl, or optionally R₄ and R₄′ together form a heterocyclic ring.

19. The method of claim 13, wherein R₇” is selected from the group consisting of NR₈R₈′ or OR₈.

20. The method of claim 13, wherein R₈, and R₈′ are each independently selected from the group consisting of H, alkyl, or optionally R₈ and R₈′ together form a heterocyclic ring.

21. The method of claim 13, wherein R₅, R₅′, R₆, R₇, and R₇′ are each independently selected from the group consisting of H, alkyl, or aryl.

22. The method of claim 13, wherein n and p are each independently 0 and R₄ and R₇ form a direct bond between Y and Z.

23. The method of claim 13, wherein R₁, R₂, R₂′, R₃ and R₃′ are H; X is O; Y is O or NR₄′; n and p are each 1; R₇″ is selected from the group consisting of H, NR₈R₈′ or OR₈; and R₄, R₄′, R₅, R₅′, R₆, R₇, R₇′, R₈, and R₈′ are each independently selected from the group consisting of H or C₁-C₆ alkyl.

24. The method of claim 13, wherein R₁, R₂, R₂′, R₃ and R₃′ are H; X is O; Y is selected from the group consisting of O or NR₄′; n and p are each 0 and R₄ and R₇ form a direct bond between Y and Z; R₇″ is H, NR₈R₈′ or OR₈; and R₄′, R₅, R₅′, R₆, R₇′, R₈, and R₈′ are each independently H or C₁-C₆ alkyl.

25. The method of claim 13, wherein R₄ and R₄′ are each ethyl.

26. The method of claim 13, wherein the dual acting SNRI-NMDA antagonist is a single enantiomer.

27. The method of claim 1, wherein the dual acting SNRI-NMDA antagonist is represented by the following structural formula:

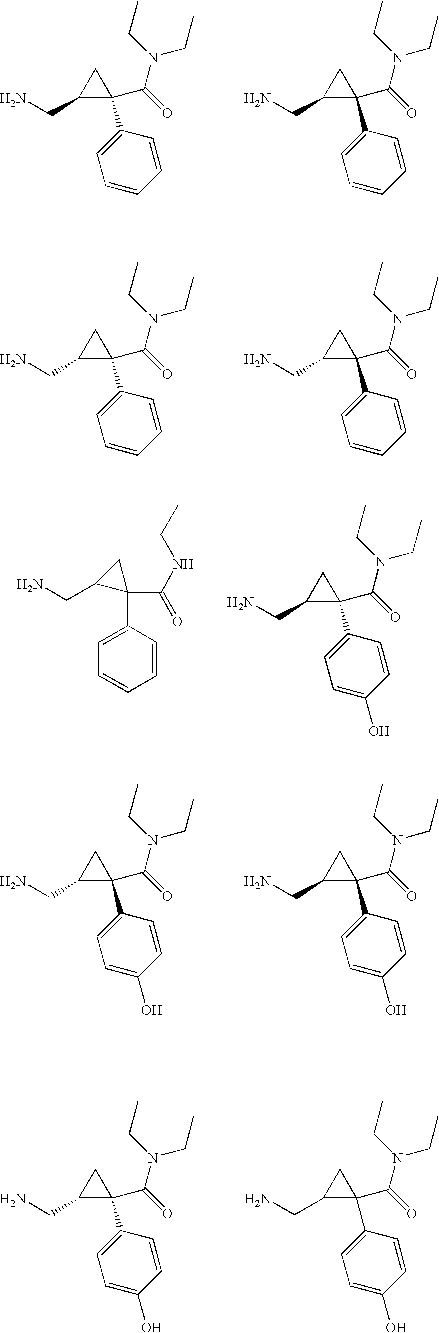

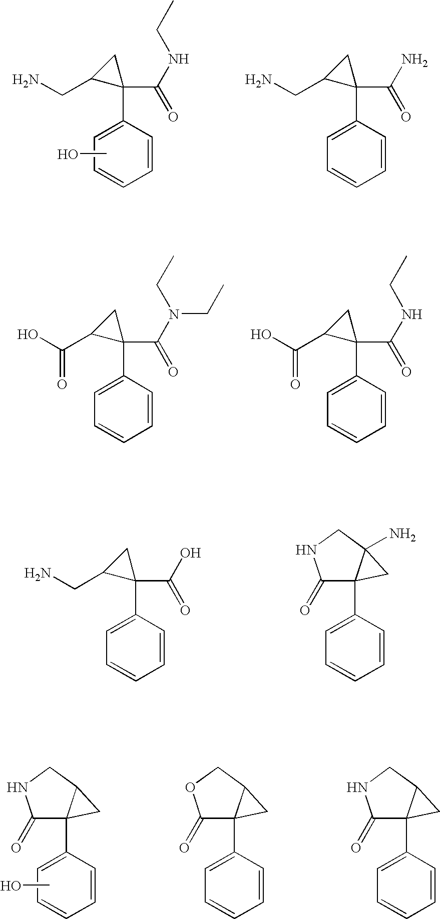

28. The method of claim 1, wherein the dual acting SNRI-NMDA antagonist is a compound of Formula III: 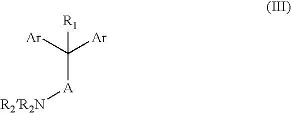
wherein:
Ar are each independently selected from the group consisting of cycloalkyl, aryl, aralkyl, heteroaryl and heteroaralkyl group, optionally Ar is substituted with one or more amino, alkylamino, dialkylamino, alkyl, hydroxyl, alkoxy, mercapto, alkylthio, alkylsulfinyl, acyl, halogen, perhaloalkyl, trifluoromethyl, trifluoromethylthio, trifluoromethylsulfonyl, and/or trifluoromethoxy;
optionally each Ar is taken together to form a fused polycyclic ring system;
R₁ is selected from the group consisting of H, alkyl, aryl and aralkyl;
R₂ and R₂′ are each independently selected from the group consisting of H, alkyl, alkylaryl, acyl;
A is selected from the group consisting of an alkylene, an alkenylene and an alkynylene, optionally interrupted with —O—, —S—, or —NH—; and
optionally R₂ and/or R₂′ are taken together with A to form a heterocycle or a heteroaryl.
or pharmaceutically acceptable salts thereof.

29. The method of claim 28, wherein:
Ar are each independently selected from the group consisting of phenyl, phenoxy, benzyl, naphthyl, thiofuranyl, tetrahydronaphthyl, pyridyl, quinolinyl, isoquinolinyl, tetrahydroquinolinyl, tetrahydroisoquinolinyl, cyclohexyl, cycloheptyl and cyclopentyl, optionally Ar is substituted with one or more amino, alkylamino, dialkylamino, alkyl, hydroxyl, alkoxy, mercapto, alkylthio, alkylsulfinyl, acyl, halogen, perhaloalkyl, trifluoromethyl, trifluoromethylthio, trifluoromethylsulfonyl, and/or trifluoromethoxy;
optionally each Ar is taken together to form a fused polycyclic ring system selected from the group consisting of dibenzo[7]annulene, a dihydrodibenzo[7]annulene, a xanthenyl and a thioxanthenyl;
R₁ is selected from the group consisting of H and alkyl;
R₂ and R₂′ are each independently selected from the group consisting of H and alkyl;
A is a liner or branched alkylene or alkenylene, optionally interrupted with an —O—; and
optionally R₂ and/or R₂′ are taken together with A to form a heterocycle.

30. The method of claim 28, wherein the dual acting SNRI-NMDA antagonist is a single enantiomer.

31. The method of claim 1, wherein the dual acting SNRI-NMDA antagonist is at least one member selected from the group consisting of compounds represented by the following structural formulas:

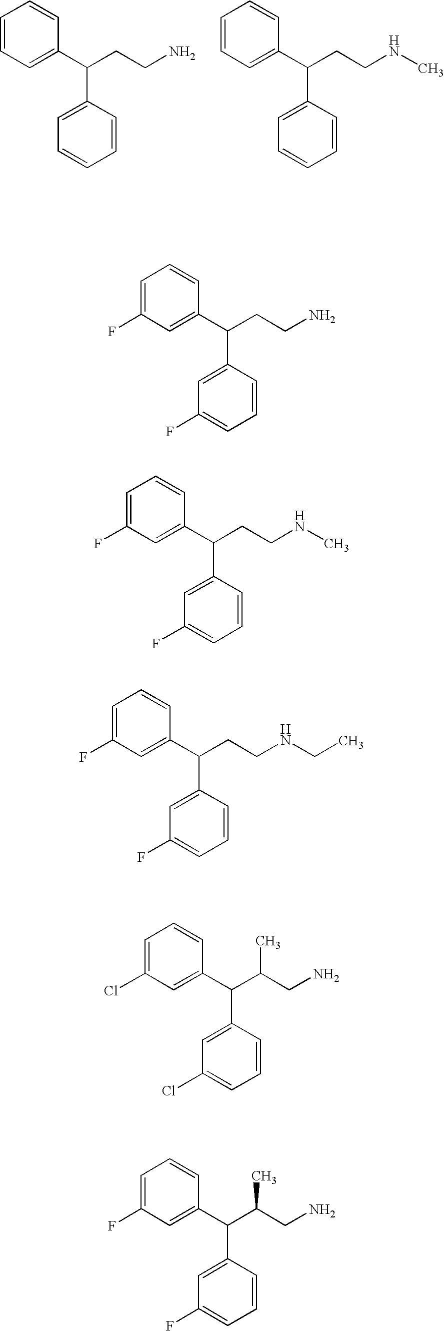

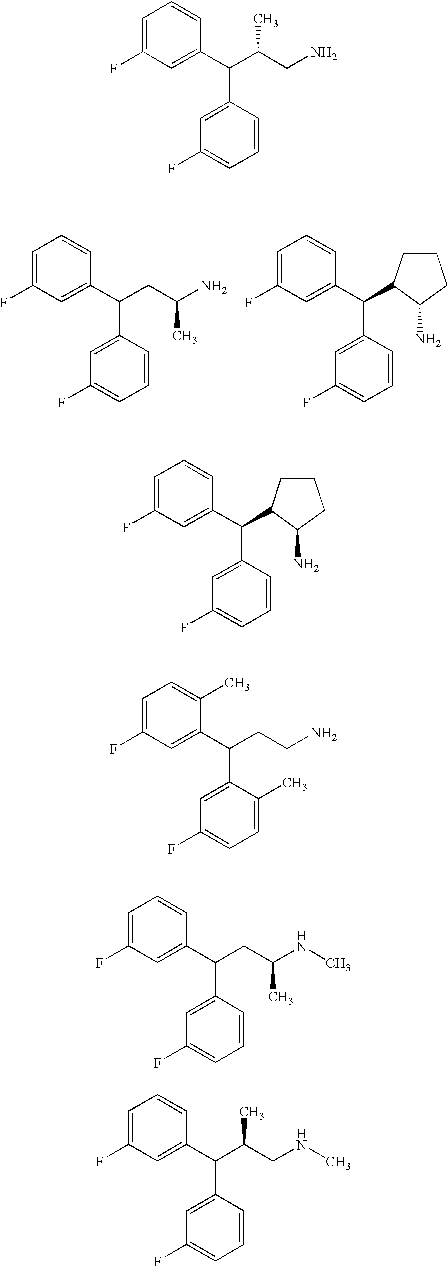

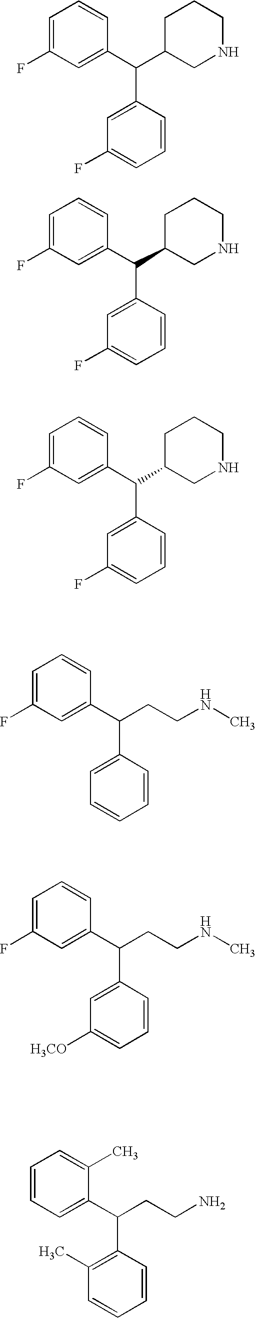

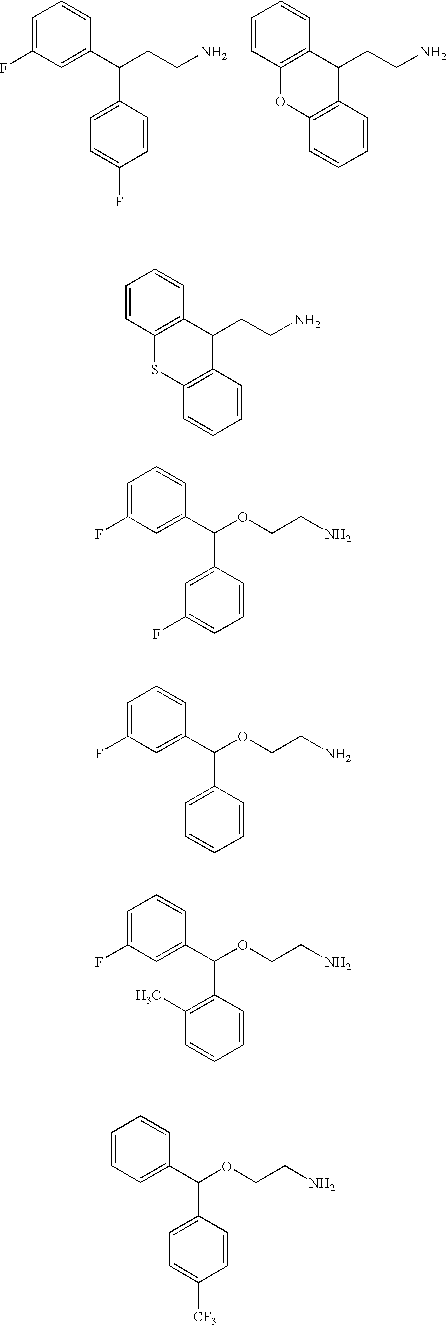

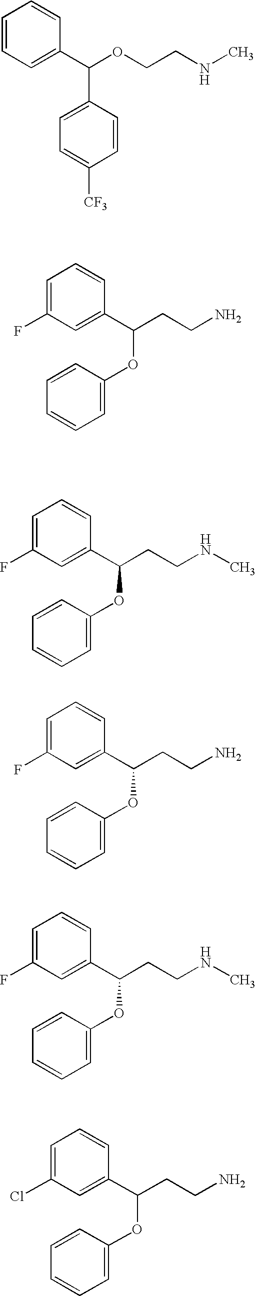

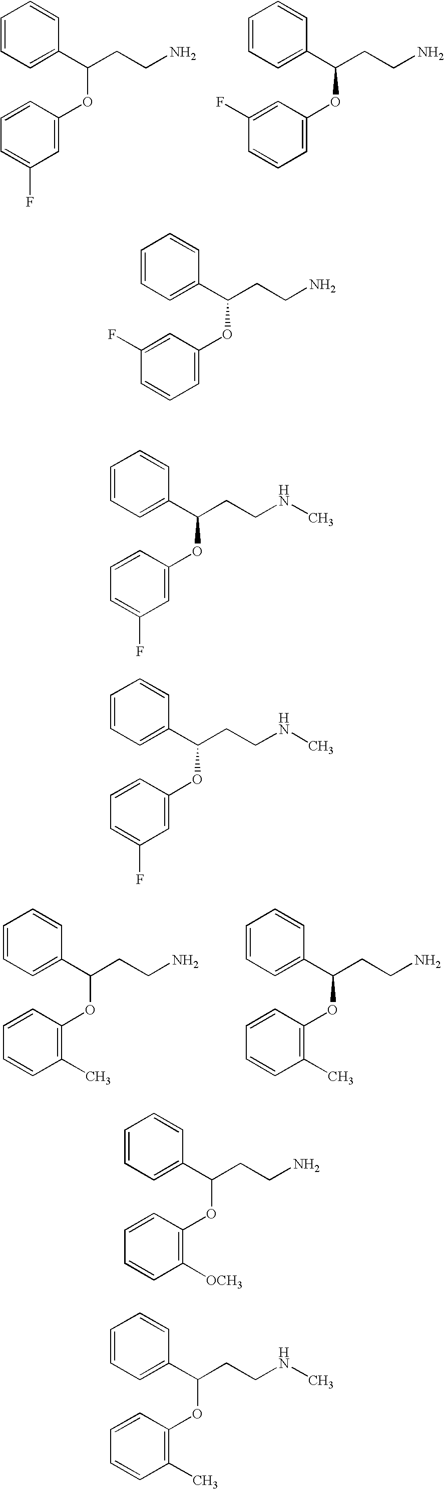

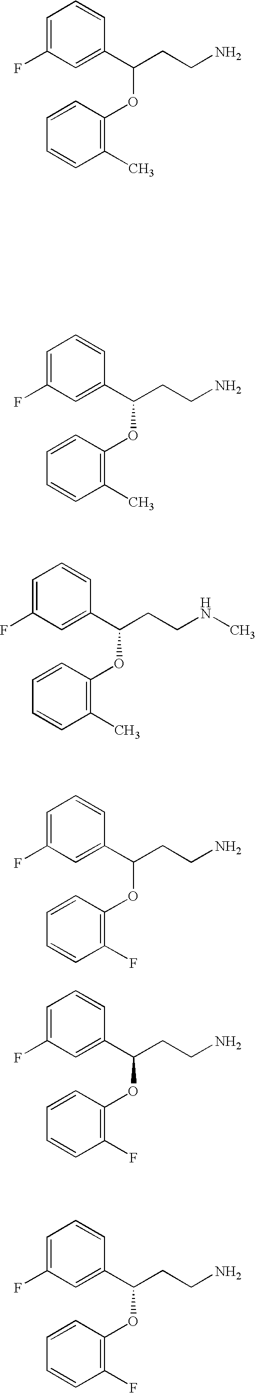

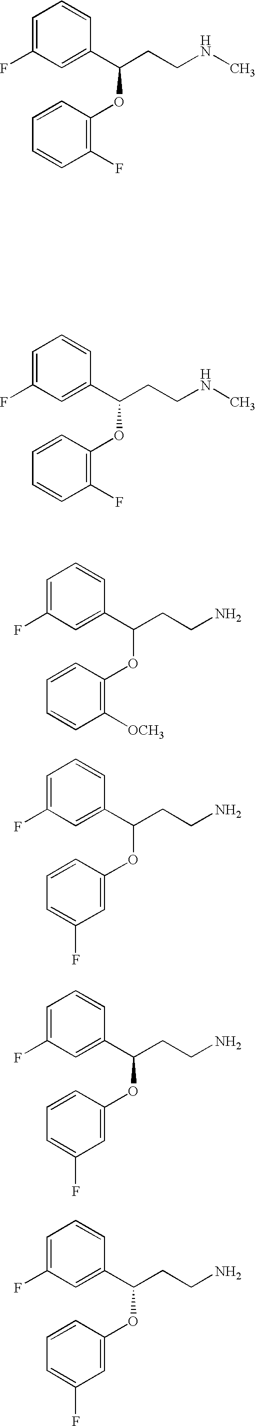

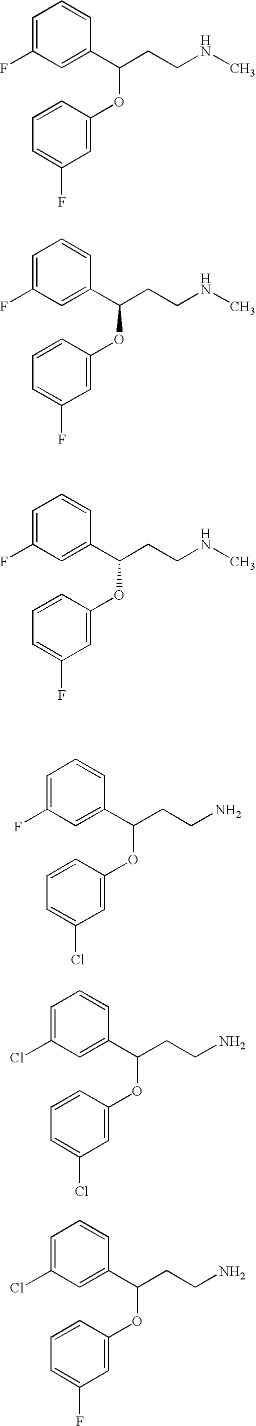

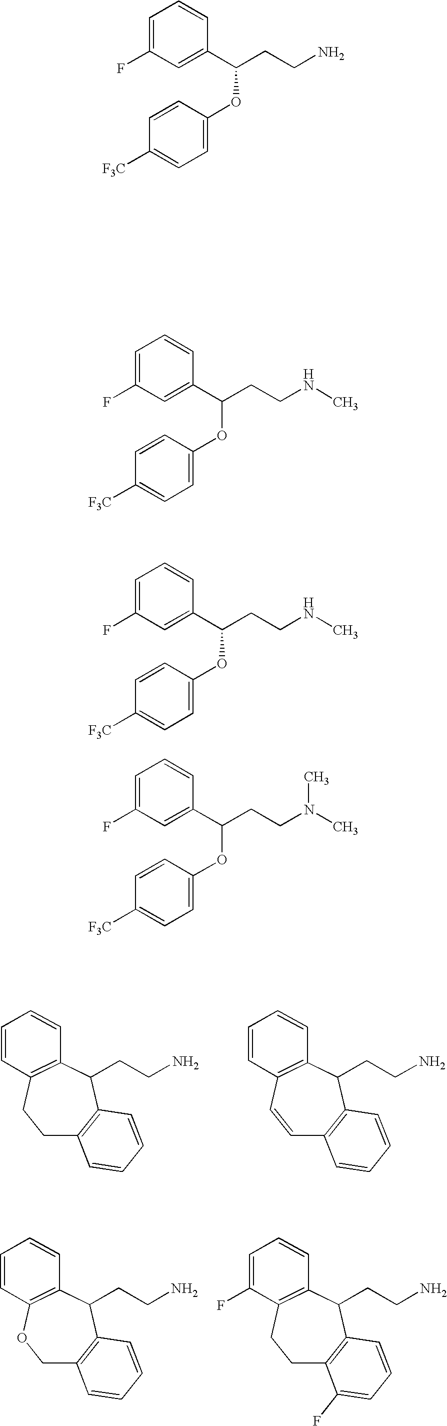

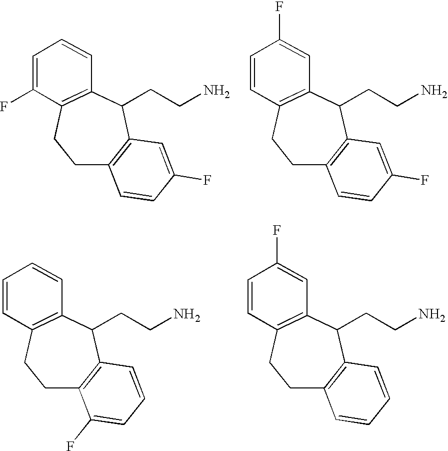

32. The method of claim 1, wherein the genitourinary disorder is associated with control of the smooth muscle of the urinary bladder.

33. The method of claim 1, wherein the genitourinary disorder is at least one disorder selected from the group consisting of overactive bladder, overactive bladder with sphincter dysfunction, urinary incontinence, urge urinary incontinence, stress urinary incontinence, Fowler's Syndrome, outlet obstruction, outlet insufficiency, pelvic hypersensitivity, sphincteric spasticity, detrusor hyperreflexia (neurogenic bladder), detrusor instability, benign prostatic hyperplasia (BPH), urethral stricture disease, tumors, interstitial (cell) cystitis, chronic pelvic pain syndrome, prostatodynia, prostatis, vulvodynia, vulvar vestibulitis, urethritis, and orchidalgia.

34. The method of claim 1, wherein the genitourinary disorder is characterized by bladder-sphincter dyssynergia.

35. The method of claim 1, wherein the genitourinary disorder is a genitourinary disorder with outlet obstruction.

36. The method of claim 1, wherein the genitourinary disorder is at least one disorder selected from the group consisting of dry overactive bladder, overactive bladder with sphincter dysfunction, urge urinary incontinence, Fowler's Syndrome, chronic pelvic pain syndrome, prostatitis, prostatodynia, vulvodynia, vestibulitis, and benign prostatic hyperplasia.

37. The method of claim 1, wherein the genitourinary disorder is urge urinary incontinence.

38. The method of claim 1, wherein the subject is a human.

39. The method of claim 1, wherein the subject does not have a chemical dependency.

40. The method of claim 1, wherein the dual acting SNRI-NMDA antagonist is administered on an as-needed basis.

41. The method of claim 40, wherein the dual acting SNRI-NMDA antagonist is administered prior to commencement of an activity wherein suppression of a genitourinary disorder is desired.

42. The method of claim 40, wherein the dual acting SNRI-NMDA antagonist is administered from about 0 minutes to about 10 hours prior to commencement of an activity wherein suppression of a genitourinary disorder is desired.

43. The method of claim 40, wherein the dual acting SNRI-NMDA antagonist is administered from about 0 minutes to about 3 hours prior to commencement of an activity wherein suppression of a genitourinary disorder is desired.

44. The method of claim 1, wherein the dual acting SNRI-NMDA antagonist is administered in a controlled release formulation.

45. The method of claim 44, wherein the dual acting SNRI-NMDA antagonist is administered in a delayed release formulation.

46. The method of claim 44, wherein the dual acting SNRI-NMDA antagonist is administered in a pulsatile release formulation.

47. The method of claim 44, wherein the dual acting SNRI-NMDA antagonist is administered in a sustained release formulation.

48. The method of claim 1, wherein the dual acting SNRI-NMDA antagonist is administered orally.

49. The method of claim 37, wherein the dual acting SNRI-NMDA antagonist is administered in at least one dosage form selected from the group consisting of: a tablet, a capsule, a caplet, a pill, a gel cap, a troche, a lozenge, a magma, a dispersion, a solution, a suspension, a syrup, a granule, a bead, a powder and a pellet.

50. The method of claim 49, wherein the dosage form is a tablet.

51. The method of claim 49, wherein the dosage form is a capsule.

52. The method of claim 1, wherein the dual acting SNRI-NMDA antagonist is administered transmucosally.

53. The method of claim 52, wherein the dual acting SNRI-NMDA antagonist is administered sublingually.

54. The method of claim 52, wherein the dual acting SNRI-NMDA antagonist is administered bucally.

55. The method of claim 52, wherein the dual acting SNRI-NMDA antagonist is administered transurethrally.

56. The method of claim 52, wherein the dual acting SNRI-NMDA antagonist is administered rectally.

57. The method of claim 1, wherein the dual acting SNRI-NMDA antagonist is administered by inhalation.

58. The method of claim 1, wherein the dual acting SNRI-NMDA antagonist is administered intravesically.

59. The method of claim 1, wherein the dual acting SNRI-NMDA antagonist is administered topically.

60. The method of claim 1, wherein the dual acting SNRI-NMDA antagonist is administered transdermally.

61. The method of claim 1, wherein the dual acting SNRI-NMDA antagonist is administered parenterally.

62. The method of claim 1, wherein the heart rate of the subject is not increased by more than 50%.

63. The method of claim 1, wherein the heart rate of the subject is not increased by more than 25%.

64. The method of claim 1, wherein the heart rate of the subject is not increased by more than 10%.

65. The method of claim 1, wherein the arterial pressure of the subject is not increased by more than 25%.

66. The method of claim 1, wherein the arterial pressure of the subject is not increased by more than 10%.

67. The method of claim 1, further comprising administering to the subject a therapeutically effective amount of at least one additional agent selected from the group consisting of an antimuscarinic, oxybutynin, DITROPAN®, tolterodine, flavoxate, propiverine, trospium, a muscosal surface protectant, ELMIRON®, an antihistamine, hydroxyzine hydrochloride, pamoate, an anticonvulsant, NEURONTIN®, KLONOPIN®, a muscle relaxant, VALIUM®, a bladder antispasmodic, URIMAX®, a tricyclic antidepressant, imipramine, a nitric oxide donor, nitroprusside, a β₃-adrenergic receptor agonist, a bradykinin receptor antagonist, a neurokinin receptor antagonist, a sodium channel modulator, such as TTX-R sodium channel modulator and/or activity dependent sodium channel modulator and a Cav2.2 subunit calcium channel modulator.

68. A method of treating a genitourinary disorder in a subject in need of treatment, comprising administering to a subject in need of treatment a therapeutically effective amount of at least one dual acting SNRI-NMDA antagonist selected from the group consisting of compounds with the following structural formulas: 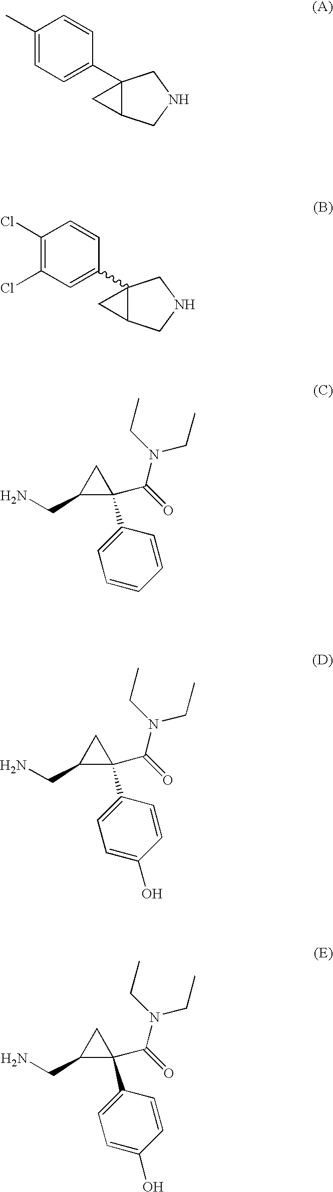 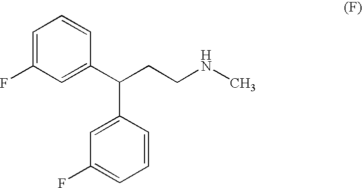
or pharmaceutically acceptable salts thereof, such that the genitourinary disorder is treated.

69. The method of claim 68, wherein the dual acting SNRI-NMDA antagonist is represented by the following structural formula:

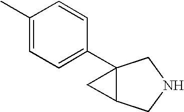

70. The method of claim 69, wherein the dual acting SNRI-NMDA antagonist is administered in a dose of 200 mg.

71. The method of claim 69, wherein the dual acting SNRI-NMDA antagonist is administered in a dose of 400 mg.

72. The method of claim 69, wherein the dual acting SNRI-NMDA antagonist is administered in a dose of 600 mg.

73. The method of claim 68, wherein the dual acting SNRI-NMDA antagonist is represented by the following structural formula:

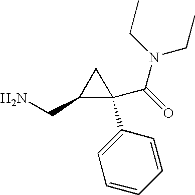

74. The method of claim 73, wherein the dual acting SNRI-NMDA antagonist is administered in a dose of between 30 mg and 200 mg.

75. The method of claim 68, wherein the genitourinary disorder is associated with control of the smooth muscle of the urinary bladder.

76. The method of claim 68, wherein the genitourinary disorder is at least one disorder selected from the group consisting of overactive bladder, overactive bladder with sphincter dysfunction, urinary incontinence, urge urinary incontinence, stress urinary incontinence, Fowler's Syndrome, outlet obstruction, outlet insufficiency, pelvic hypersensitivity, sphincteric spasticity, detrusor hyperreflexia (neurogenic bladder), detrusor instability, benign prostatic hyperplasia (BPH), urethral stricture disease, tumors, interstitial (cell) cystitis, chronic pelvic pain syndrome, prostatodynia, prostatis, vulvodynia, vulvar vestibulitis, urethritis, and orchidalgia.

77. The method of claim 68, wherein the genitourinary disorder is characterized by bladder-sphincter dyssynergia.

78. The method of claim 68, wherein the genitourinary disorder is a genitourinary disorder with outlet obstruction.

79. The method of claim 68, wherein the genitourinary disorder is at least one disorder selected from the group consisting of dry overactive bladder, overactive bladder with sphincter dysfunction, urge urinary incontinence, Fowler's Syndrome, chronic pelvic pain syndrome, prostatitis, prostatodynia, vulvodynia, vestibulitis, and benign prostatic hyperplasia.

80. The method of claim 68, wherein the genitourinary disorder is urge urinary incontinence.

81. The method of claim 68, wherein the subject is a human.

82. The method of claim 68, wherein the subject does not have a chemical dependency.

83. A method of treating overactive bladder in a subject comprising administering to the subject a therapeutically effective amount of a dual acting SNRI-NMDA antagonist, such that the overactive bladder is treated.

84-87. (canceled)

88. A method of treating overactive bladder in a subject in need of treatment, comprising administering to said subject a therapeutically effective amount of at least one dual acting SNRI-NMDA antagonist selected from the group consisting of compounds with the following structural formulas: 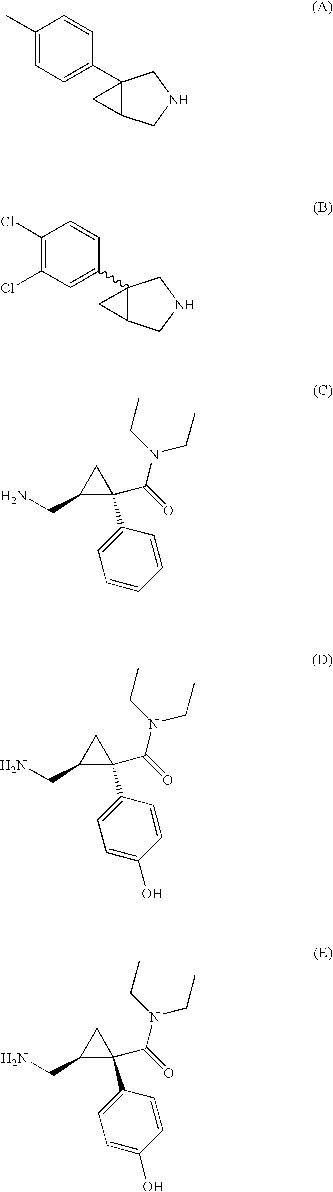 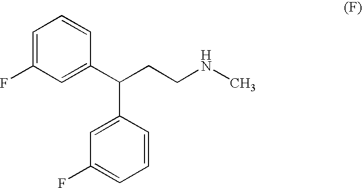
or pharmaceutically acceptable salts thereof, such that the overactive bladder is treated.

89-90. (canceled)

91. A kit for treating a genitourinary disorder in a subject in need of treatment, comprising:
a dual acting SNRI-NMDA antagonist, packaged with an instructional insert for using the dual acting SNRI-NMDA antagonist for the treatment of the genitourinary disorder.

92-94. (canceled)

95. A kit for treating a genitourinary disorder in a subject in need of treatment, comprising:
an SNRI packaged with an instructional insert for using the SNRI together with an NMDA antagonist for the treatment of a genitourinary disorder.

96. (canceled)

97. A kit for treating a genitourinary disorder in a subject in need of treatment, comprising:
an NMDA antagonist packaged with an instructional insert for using the NMDA antagonist together with an SNRI for the treatment of a genitourinary disorder.

98-107. (canceled)
